(19) United States
(12) Patent Application Publication
(10) Pub. No.: US 2024/0018513 A1
(43) Pub. Date: Jan. 18, 2024

(54) SYNTHETIC INTRONS FOR TARGETED GENE EXPRESSION

(71) Applicants: FRED HUTCHINSON CANCER CENTER, Seattle, WA (US); MEMORIAL SLOAN KETTERING CANCER CENTER, New York, NY (US)

(72) Inventors: Robert K. Bradley, Seattle, WA (US); Omar Abdel-Wahab, New York, NY (US); Khrystyna North, Seattle, WA (US); Salima Benbarche, New York, NY (US); Bo Liu, New York, NY (US)

(73) Assignees: FRED HUTCHINSON CANCER CENTER, Seattle, WA (US); MEMORIAL SLOAN KETTERING CANCER RESEARCH CENTER, New York, NY (US)

(21) Appl. No.: 18/249,914

(22) PCT Filed: Oct. 22, 2021
(86) PCT No.: PCT/US2021/056273

§ 371 Date: Apr. 20, 2023

Related U.S. Application Data
(60) Provisional application No. 63/160,405, filed on Mar. 12, 2021.
(60) Provisional application No. 63/105,143, filed on Oct. 23, 2020.

(51) Int. Cl. C12N 15/10

(52) U.S. Cl. CPC C12N 15/1082; C12N 15/1051

(57) ABSTRACT

The disclosure provides artificial nucleic acid introns configured for selective splicing in cells with aberrant RNA splicing activity, e.g., neoplastic cells. The artificial intron can comprise a 5′ splice site, a canonical 3′ splice site, at least one cryptic 3′ splice site, a pyrimidine-rich domain, and at least one branchpoint. Also provided are constructs integrating the artificial introns with exons in a configuration that, when the artificial intron is spliced out by the aberrant RNA splicing factors, encode a functional protein. Also disclosed are methods that employ the disclosed platform of selective expression, including, targeted gene therapy methods (e.g., in cancers), diagnostics and imaging, and drug screening.

### CROSS-REFERENCES TO RELATED APPLICATIONS

This application claims the benefit of U.S. Provisional Application Nos. 63/105,143, filed Oct. 23, 2020, and 63/160,405, filed Mar. 12, 2021, the disclosures of which are hereby expressly incorporated herein by reference in their entireties.

### STATEMENT OF GOVERNMENT LICENSE RIGHTS

This invention was made with Government support under HL128239, DK103854, and CA251138 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 1896-P40WO_Seq_List_FINAL_20211018_ST25.txt. The text file is 80 KB; was created on Oct. 18, 2021; and is being submitted via EFS-Web with the filing of the specification.

### BACKGROUND

A number of studies have tried to use gene therapy, i.e., the introduction of novel fgenetic material into cells, as a novel modality for the treatment of cancers. See, e.g., Amer, M., Gene therapy for cancer: present status and future perspective, Mol Cell Ther. 2014; 2: 27. Unfortunately, these studies have not achieved the desired clinical benefits. One major challenge for developing gene therapy for cancer treatment is that accidental delivery of the gene therapy payload to healthy normal cells can result in unintended and adverse side effects. For example, if the payload was a “killer gene” that triggered cancer cell apoptosis, then delivery of this payload to healthy cells could result in their unwanted deaths leading to potentially severe side-effects. As a consequence, developing a reliable and generalizable method to permit expression of a given gene or protein in cancer cells, but not normal cells, or alternately in normal cells but not cancer cells, would be a major and important step toward bringing gene therapy for cancers into the clinic.

The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides an artificial nucleic acid construct comprising an intron. The intron comprises: a 5′ splice site; a canonical 3′ splice site; at least one cryptic 3′ splice site, that is within about 100 nucleotides upstream of the canonical 3′ splice site or within about 50 nucleotides downstream of the canonical 3′ splice site; a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and at least one branchpoint at least 15 nucleotides upstream of the canonical 3′ splice site. In some embodiments, the intron is at least about 50 nucleotides to about 1000 nucleotides in length.

In some embodiments, the intron is derived from a human wildtype intron selected from intron 1 of MTERFD3, intron 4 of MYO15B, intron 10 of SYTL1, intron 11 of SYTL1, intron 4 of MAP3K7, intron 1 of ORAI2, and intron 1 of TMEM14C. In some embodiments, the human wildtype intron from which the intron is derived is one of the following: intron 1 of MTERFD3 comprising a sequence set forth in SEQ ID NO:2; intron 4 of MYO15B comprising a sequence set forth in SEQ ID NO:8; intron 10 of SYTL1 comprising a sequence set forth in SEQ ID NO:13; intron 11 of SYTL1 comprising a sequence set forth in SEQ ID NO:15; intron 4 of MAP3K7 comprising a sequence set forth in SEQ ID NO:22; intron 1 of ORAI2 comprising a sequence set forth in SEQ ID NO:26; and intron 1 of TMEM14C comprising a sequence set forth in SEQ ID NO:30. In some embodiments, the intron is derived from a human wildtype intron 1 of MTERFD3, and wherein the intron further comprises one, two, three, or more of the following features: a 5′ splice site comprising a GT dinucleotide immediately followed by a consensus 5′ splice site context, optionally wherein the consensus 5′ splice site context includes one of AAG, GAG, GTG, and the like; a canonical 3′ splice site comprising an AG dinucleotide immediately preceded by a C or T; at least one cryptic 3′ splice site, located at least 5 nucleotides upstream of the canonical 3′ splice site, with an AG dinucleotide and comprising a sequence that is a weaker 3′ splice site than is the canonical 3′ splice site, where splice site strength is estimated with the MaxEntScan algorithm or similar methods; a pyrimidine-rich domain comprising at least 15 consecutive nucleotides, wherein the sequence of the pyrimidine-rich domain is at least 60% pyrimidine nucleotides and at least 40% thymine nucleotides, and wherein the pyrimidine-rich domain is within at least 30 nucleotides of a cryptic 3′ splice site; and at least one branchpoint at least 20 nucleotides upstream of the canonical 3′ splice site. In some embodiments, the intron has a 5′ end domain with about 10 to about 150 nucleotides having at least 50% sequence identity to a sequence of the 5′-most 10 to about 150 nucleotides of the wildtype intron. In some embodiments, the intron has a 3′ end domain with about 50 to about 350 nucleotides having at least 50% sequence identity to a sequence of the 3′-most 50 to about 350 nucleotides of the wildtype intron. In some embodiments, the intron has a sequence with at least 75% sequence identity to a sequence selected from SEQ ID NOS:4-6, 10, 11, 17-20, 24, 28, 32, and 150-157.

In some embodiments, the 5′ splice site comprises a sequence selected from GTGAG, GTAAG, GTGCG, GTACG, GTGGG, GTAGG, GTGTG, GTATG, and GTATC. In some embodiments, the canonical 3′ splice site comprises a sequence selected from AAG, CAG, and TAG. In some embodiments, the at least one cryptic 3′ splice site comprises a sequence selected from AAG, CAG, GAG, TAG, ATG, CTG, GTG, and TTG. In some embodiments, the intron comprises a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites comprises a sequence independently selected from AAG, CAG, GAG, TAG, ATG, CTG, GTG, and TTG.

In some embodiments, the pyrimidine-rich domain is characterized by one, two, three, or all of the following: wherein the pyrimidine-rich domain comprises at least 15 consecutive nucleotides; wherein the pyrimidine-rich domain has a sequence with at least 60% pyrimidine nucleotides and is at least 40% thymine nucleotides; wherein the pyrimidine-rich domain is within at least 30 nucleotides of a cryptic 3′ splice site; and wherein the pyrimidine-rich domain has a sequence with at least 50% sequence identity to any 20 nucleotides selected from the sequence set forth as SEQ ID NO:49.

In some embodiments, the at least one branchpoint is at least 20 nucleotides upstream of the canonical 3′ splice site, and wherein the branchpoint nucleotide is an adenine. In some embodiments, the branchpoint and surrounding sequence context has sequence identity of at least 60% to the sequence tactaAca, where the uppercase A is the branchpoint nucleotide.

In some embodiments, the intron is configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene. In some embodiments, the RNA splicing factor gene is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, and combinations thereof, with reference to the wild-type amino acid sequence set forth in SEQ ID NO:190.

In some embodiments, the nucleic acid construct further comprises a first exon domain and a second exon domain, wherein the intron is disposed between the first exon domain and the second exon domain. In some embodiments, the combination of the first exon domain and the second exon domain without the intron encodes part or all of a protein of interest. In some embodiments, the nucleic acid intron construct comprises an expression cassette comprising the first exon domain, the intron, the second exon domain, and a promoter sequence operatively linked thereto.

In another aspect, the disclosure provides a method of generating an artificial nucleic acid construct with an intron, e.g., an artificial intron. The method comprises:
(1) ligating a 5′ end domain of a human wildtype intron to a 3′ end domain of the human wildtype intron to provide an abbreviated intron that lacks an interior sequence, wherein the 5′ end domain comprises about 10 to about 150 nucleotides of the 5′ end sequence of the human wildtype intron, and wherein the 3′ end domain comprises about 50 to about 350 nucleotides of the 3′ end sequence of the human wildtype intron;
(2) implementing one or more sequence modifications to the abbreviated intron sequence to provide a first plurality of artificial introns derived from the abbreviated intron sequence;
(3) selecting artificial introns from the first plurality of artificial introns that conform to at least three of the following parameters:
a 5′ splice site;
a canonical 3′ splice site;
at least one cryptic 3′ splice site, that is within about 100 nt nucleotides upstream of the canonical 3′ splice site or within about 50 nt nucleotides downstream of the canonical 3′ splice site;
a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and
at least one branchpoint at least 15 nucleotides upstream of the canonical 3′ splice site.

In some embodiments, the human wildtype intron is selected from intron 1 of MTERFD3, intron 4 of MYO15B, intron 10 of SYTL1, intron 11 of SYTL1, intron 4 of MAP3K7, intron 1 of ORAI2, intron 1 of TMEM14C, or functional variants thereof. In some embodiments, the human wildtype intron is one of the following: intron 1 of MTERFD3 comprising a sequence set forth in SEQ ID NO:2; intron 4 of MYO15B comprising a sequence set forth in SEQ ID NO:8; intron 10 of SYTL1 comprising a sequence set forth in SEQ ID NO: 13; intron 11 of SYTL1 comprising a sequence set forth in SEQ ID NO:15; intron 4 of MAP3K7 comprising a sequence set forth in SEQ ID NO:22; intron 1 of ORAI2 comprising a sequence set forth in SEQ ID NO:26; and intron 1 of TMEM14C comprising a sequence set forth in SEQ ID NO:30.

In some embodiments, the one or more sequence modifications comprises one or more of the following in any combination or order: (a) mutating a single nucleotide; (b) mutating any pair of nucleotides within 10 nucleotides of the 5′ end of the abbreviated intron sequence or 30 nucleotides of the 3′ end of the abbreviated intron sequence; (c) deleting any consecutive stretch of 5, 10, 15, 20, 25, 30, 35, 40, 45, 50, 100, 125, 150, 200, or 250 nucleotides; (d) mutating any pair of nucleotides within the 5 nt nucleotides upstream of and 2 nucleotides downstream of each branchpoint; (e) mutating any combination of branchpoints to guanine; (f) mutating any combination of multiple adenines to guanines; (g) mutating any combination of branchpoint contexts to strong branchpoint contexts, optionally wherein the strong branchpoint context comprises a sequence with a sequence identity of at least 50% to the sequence tactaAca, where A is a branchpoint nucleotide and tacta_ca is a context sequence; (h) mutating any four consecutive nucleotides to cAGg; (i) inserting a polypyrimidine tract immediately followed by a 3′ splice site at any position; (j) mutating any consecutive stretch of nucleotides to one or more thymines; (k) mutating all pyrimidines within any six or more consecutive positions to guanines; (l) inserting a strong branchpoint and flanking sequence context at any position; (m) inserting one or more intronic splicing enhancers at any position; and (n) inserting one or more intronic splicing silencers at any position.

In some embodiments, the polypyrimidine tract immediately followed by a 3′ splice site comprises at least 6 consecutive nucleotides containing at least 4 pyrimidines, immediately followed by a sequence selected from AAG, CAG, GAG, TAG, ATG, CTG, GTG, or TTG, and the like. In some embodiments, the strong branchpoint and flanking sequence context comprises a sequence with a sequence identity of at least 50% to the sequence tactaAca, where uppercase indicates the branchpoint, and the like. In some embodiments, the one or more intronic splicing enhancers are selected from GGGTTT, GGTGGT, TTTGGG, GAGGGG, GGTATT, GTAACG, and the like. In some embodiments, the one or more intronic splicing silencers are selected from CACACCA, CTCCTC, TACAGCT, CTTCAG, GAACAG, CAAAGGA, AGATATT, ACATGA, AATTTA, AGTAGG, and the like.

In another aspect, the disclosure provides an artificial nucleic acid intron construct produced by the method disclosed herein.

In another aspect, the disclosure provides a method of modifying a nucleic acid sequence to permit selective expression, or alternately selective lack of expression, in a cell characterized by a mutation in an RNA splicing factor gene. The method comprises: (1) providing a sequence of a target nucleic acid molecule and sequence of an artificial nucleic acid intron as described herein, wherein the artificial nucleic acid intron is derived from a wildtype intron with known nucleotide sequences of upstream and downstream flanking exons; (2) identifying one or more dinucleotides in the target nucleic acid sequence that are identical to an intron dinucleotide sequence consisting of the 3′-most nucleotide of the upstream exon flanking the wildtype intron and the 5′-most nucleotide of the downstream exon flanking the wildtype intron; (3) selecting a dinucleotide identified in step (2) as an insertion point, wherein the insertion point divides the target nucleic acid into a first domain and a second domain, optionally wherein one of the first domain and second domain is at least about 50% of the length of the other of the first domain and second domain; and (4) inserting an artificial intron molecule with the artificial nucleic acid intron sequence between the first domain and the second domain of the target nucleic acid molecule. In some embodiments, step (3) further comprises: computationally inserting the sequence of the artificial nucleic acid intron at the selected insertion point to create a hypothetical exonic flanking sequence context for a 5′ splice site and a 3′-most 3′ splice site; computing strength scores for the 5′ splice site and the 3′-most 3′ splice site, respectively, in their hypothetical exonic contexts; comparing the computed strength scores for the 5′ splice site and 3′-most 3′ splice site within their hypothetical exonic contexts to strength scores of the respective 5′ splice site and 3′-most 3′ splice site of the wildtype intron in its wildtype exonic context from which the artificial nucleic acid intron is derived; and selecting a dinucleotide wherein computational insertion of the artificial nucleic acid intron sequence results in strength scores for the 5′ splice site and 3′-most 3′ splice site in their hypothetical exonic contexts that differ by about 50% or less of the respective 5′ splice site and 3′-most 3′ splice site scores of the wildtype intron in its wildtype exonic context. In some embodiments, strength scores are computed with a standard method such as MaxEntScan::scores5ss, MaxEntScan::score3ss, HumanSplicingFinder, and other similar algorithms.

In some embodiments, the method further comprises introducing one or more synonymous codon mutations into the nucleic acid that improve or weaken one or both scores for the 5′ splice site and/or 3′-most 3′ splice site in their hypothetical exonic contexts.

In some embodiments, the method further comprises introducing one or more synonymous codon mutations into the nucleic acid that result in creation of one or more exonic splicing enhancers. In some embodiments, the one or more exonic splicing enhancers is/are selected from CCNG, CGNG, GCNG, and GGNG, where N is any nucleotide, and other sequences with enhanced likelihood of binding by serine/arginine-rich (SR) proteins.

In some embodiments, the method further comprises introducing one or more synonymous codon mutations into the nucleic acid that result in creation of one or more exonic splicing silencers. In some embodiments, the one or more exonic splicing silencers is/are selected from TTTGTTCCGT (SEQ ID NO:160), GGGTGGTTTA (SEQ ID NO:161), GTAGGTAGGT (SEQ ID NO:162), TTCGTTCTGC (SEQ ID NO:163), GGTAAGTAGG (SEQ ID NO:164), GGTTAGTTTA (SEQ ID NO:165), TTCGTAGGTA (SEQ ID NO:166), GGTCCACTAG (SEQ ID NO:167), TTCTGTTCCT (SEQ ID NO:168), TCGTTCCTTA (SEQ ID NO:169), GGGATGGGGT (SEQ ID NO:170), GTTTGGGGGT (SEQ ID NO:171), TATAGGGGGG (SEQ ID NO:172), GGGGTTGGGA (SEQ ID NO:173), TTTCCTGATG (SEQ ID NO:174), TGTTTAGTTA (SEQ ID NO:175), TTCTTAGTTA (SEQ ID NO:176), GTAGGTTTG, GTTAGGTATA (SEQ ID NO:177), TAATAGTTTA (SEQ ID NO:178), TTCGTTTGGG (SEQ ID NO:179), and the like, or sequences with at least 50% identity thereto.

In some embodiments, two or more artificial intron molecules are inserted into the target nucleic acid resulting in a plurality of domains, optionally wherein each of the plurality of domains is at least about 50% of the length of the other domain(s). In some embodiments, the target nucleic acid molecule is an isolated nucleic acid molecule with a protein-coding sequence (CDS) that encodes a protein of interest, and the modified target nucleic acid molecule is configured to permit selective expression, or alternately selective lack of expression, in a cell characterized by a mutation in an RNA splicing factor gene.

In some embodiments, the method further comprises introducing the modified target nucleic acid molecule to a cancer cell with a mutation in an RNA splicing factor gene and permitting expression, or alternately selective lack of expression, of the protein of interest.

In some embodiments, the target nucleic acid molecule is a gene in the chromosome of a cell, wherein the gene encodes a protein of interest, and the modified target nucleic acid molecule is configured for selective expression, or alternately selective lack of expression, in a cell characterized by a mutation in an RNA splicing factor gene. In some embodiments, the cell is a cancer cell and the mutation in an RNA splicing factor gene is a change-of-function or loss-of-function mutation in a recurrently mutated RNA splicing factor gene; wherein the artificial intron sequence is configured to be spliced differently in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; wherein the different splicing pattern of the artificial intron sequence results in production of different mature transcripts of the modified target nucleic acid molecule in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; and wherein the production of different mature transcripts of the modified nucleic acid molecule permits either selective expression, or alternately selective lack of expression, of a desired protein from the target nucleic acid molecule in the cancer cell, and the opposite pattern in a cell lacking the change-of-function or loss-of-function mutation in the recurrently mutated RNA splicing factor gene. In some embodiments, the RNA splicing factor gene is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, and combinations thereof, with reference to the wild-type amino acid sequence set forth in SEQ ID NO:190.

In another aspect, the disclosure provides a method of selectively expressing, or alternately selectively not expressing, a gene of interest in a cell, wherein the cell comprises a change-of-function or loss-of-function mutation in a recurrently mutated RNA splicing factor gene. The method comprises: introducing to the cell an expression cassette comprising a coding sequence (CDS) interrupted by at least one artificial nucleic acid intron as described herein, wherein the expression cassette further comprises a promoter operatively linked to the CDS; and permitting transcription of the coding sequence and modified splicing of the transcript induced by the artificial nucleic acid intron in the resulting transcript in conjunction with the mutated splicing factor.

In some embodiments, the cell is a cancer cell and the mutation in an RNA splicing factor gene is a change-of-function or loss-of-function mutation in a recurrently mutated RNA splicing factor gene. In some embodiments, the RNA splicing factor gene is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, and combinations thereof, with reference to the wild-type amino acid sequence set forth in SEQ ID NO:190.

In some embodiments, the cancer is a myelodysplastic syndrome (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, or other neoplasm with recurrent SF3B1 mutations. In some embodiments, upon splicing of the at least one artificial nucleic acid intron from the gene transcript, the gene of interest encodes a functional therapeutic protein. In some embodiments, the functional therapeutic protein is a toxin, chemokine, cytokine, growth factor, targetable cell-surface protein, targetable antigen, druggable enzyme, detectable marker, and the like.

In another aspect, the disclosure provides a method of treating in a subject with cancer, wherein the cancer is characterized by a change-of-function or loss-of-function mutation in a recurrently mutated RNA splicing factor gene. The method comprises administering to the subject an effective amount of a therapeutic composition comprising an expression cassette comprising a coding sequence (CDS) interrupted by at least one artificial nucleic acid intron as described herein, wherein the expression cassette further comprises a promoter operatively linked to the CDS.

In some embodiments, the RNA splicing factor gene is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, and combinations thereof, with reference to the wild-type amino acid sequence set forth in SEQ ID NO: 190. In some embodiments, the cancer is selected from a myelodysplastic syndromes (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, and other neoplasm with recurrent SF3B1 mutations.

In some embodiments, upon splicing of the at least one artificial nucleic acid intron from the gene transcript in a cancer cell the CDS encodes a functional therapeutic protein. In some embodiments, the functional therapeutic protein is a toxin, chemokine, cytokine, growth factor, targetable cell-surface protein, targetable antigen, druggable enzyme, detectable marker, and the like. In some embodiments, the functional therapeutic protein is a chemokine, cytokine, or growth factor, and wherein the chemokine, cytokine, or growth factor stimulates an increased immune response against the cancer cell. In some embodiments, the functional therapeutic protein is IFN alpha, IFN beta, IFN gamma, IL-2, IL-12, IL-15, IL-18, IL-24, TNF-alpha, GM-CSF, and the like, or functional domains or derivatives thereof. In some embodiments, the functional therapeutic protein is a targetable cell-surface protein or targetable antigen, and the method further comprises administering to the subject an effective amount of a second therapeutic composition comprising an affinity reagent that specifically binds the antigen. In some embodiments, the targetable cell-surface protein or targetable antigen is CD19, CD22, CD23, CD123, ROR1, truncated EGFR (EGFRt), or functional domains thereof, and the like. In some embodiments, the second therapeutic composition comprises an antibody, or a fragment or derivative thereof, an immune cell expressing an antibody, or fragment or derivative thereof, or an immune cell expressing a T cell receptor, or fragment or derivative thereof, and wherein the antibody or T cell receptor, or fragment or derivative thereof, specifically binds the antigen. In some embodiments, the functional therapeutic protein is a toxin, wherein the toxin is optionally Caspase 9, TRAIL, Fas ligand, and the like, or functional fragments thereof. In some embodiments, the functional therapeutic protein is a druggable enzyme, optionally wherein: the druggable enzyme is herpes simplex virus thymidine kinase and the method further comprises administering to the subject an effective amount of ganciclovir; the druggable enzyme is cytosine deaminase and the method further comprises administering to the subject an effective amount of 5-fluorocytosine; the druggable enzyme is nitroreductase and the method further comprises administering to the subject an effective amount of CB1954 or analogs thereof, the druggable enzyme is carboxypeptidase G2 and the method further comprises administering to the subject an effective amount of CMDA, ZD-2767P, and the like; the druggable enzyme is purine nucleoside phosphorylase and the method further comprises administering to the subject an effective amount of 6-methylpurine deoxyriboside, and the like; the druggable enzyme is cytochrome P450 and the method further comprises administering to the subject an effective amount of cyclophosphamide, ifosfamide, and the like; the druggable enzyme is horseradish peroxidase and the method further comprises administering to the subject an effective amount of indole-3-acetic acid, and the like; or the druggable enzyme is carboxylesterase and the method further comprises administering to the subject an effective amount of irinotecan, and the like.

In some embodiments, the functional therapeutic protein is a detectable marker, and the method further comprises surgically removing the cancer cells expressing the detectable marker. In some embodiments, the expression cassette is disposed in a vector, optionally a viral vector, for intracellular delivery. In some embodiments, the viral vector is derived from AAV, adenovirus, herpes simplex virus, retrovirus, lentivirus, alphavirus, flavivirus, rhabdovirus, measles virus, Newcastle disease virus, Coxsackievirus, poxvirus, and the like.

In some embodiments, the therapeutic composition further comprises a vehicle for intracellular delivery and a pharmaceutically acceptable carrier. In some embodiments, the vehicle is a liposome, nanocapsule, nanoparticle, exosome, microparticle, microsphere, lipid particle, vesicle, and the like, configured for the introduction of the expression cassette into cancer cells.

In another aspect, the disclosure provides method of enhancing surgical resection of a tumor from a subject, wherein the tumor is characterized by a change-of-function or loss-of-function mutation in a recurrently mutated RNA splicing factor gene. The method comprises: administering to the subject an effective amount of a therapeutic composition comprising an expression cassette comprising a coding sequence (CDS) encoding a detectable marker, wherein the CDS is interrupted by at least one artificial nucleic acid intron as described herein, and wherein the expression cassette further comprises a promoter operatively linked to the CDS.

In some embodiments, the RNA splicing factor gene is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, and combinations thereof, with reference to the wild-type amino acid sequence set forth in SEQ ID NO:190.

In some embodiments, the cancer is selected from a uveal melanoma, mucosal melanoma, skin melanoma, breast cancer, pancreatic cancer, endometrial cancer, liver cancer, lung cancer, mesothelioma, or other solid tumor or neoplasm with recurrent SF3B1 mutations.

In some embodiments, the detectable marker is a fluorescent or luminescent protein. In some embodiments, the method further comprises detecting fluorescent or luminescent tumor cells and surgically resecting the fluorescent or luminescent tumor cells.

In some embodiments, the expression cassette is disposed in a vector, optionally a viral vector, for intracellular delivery. In some embodiments, the viral vector is derived from AAV, adenovirus, herpes simplex virus, retrovirus, lentivirus, alphavirus, flavivirus, rhabdovirus, measles virus, Newcastle disease virus, Coxsackievirus, poxvirus, and the like.

In some embodiments, the therapeutic composition further comprises a vehicle for intracellular delivery and a pharmaceutically acceptable carrier. In some embodiments, the vehicle is a liposome, nanocapsule, nanoparticle, exosome, microparticle, microsphere, lipid particle, vesicle, and the like, configured for the introduction of the expression cassette into cancer cells.

In another aspect, the disclosure provides a method of screening candidate compositions for activity in a cell, wherein the cell has a genetic background comprising a change-of-function or loss-of-function mutation in a recurrently mutated RNA splicing factor gene. The method comprises contacting the cell with an expression cassette comprising a coding sequence (CDS) interrupted by at least one artificial nucleic acid intron as described herein. The expression cassette further comprises a promoter operatively linked to the CDS, and wherein upon splicing of the artificial nucleic acid intron the CDS encodes or does not encode a detectable reporter protein. The specific splicing outcome depends upon mutant splicing factor activity in the cell. The method further comprises contacting the cell with a candidate composition; permitting transcription of the coding sequence; and detecting the presence or absence of a functional reporter protein.

In some embodiments, detection of a functional reporter protein or a relative increase of functional reporter protein in the cell indicates the candidate composition does not suppress activity of the mutated RNA splicing factor in the cell. Detection of an absence or relative reduction in functional reporter protein in the cell indicates the candidate composition does suppress activity of the mutated RNA splicing factor in the cell.

In some embodiments, detection of a functional reporter protein in the cell indicates the candidate composition suppresses activity of the mutated RNA splicing factor in the cell. An absence or relative reduction in detected functional reporter protein in the cell indicates the candidate composition does not suppress activity of the mutated RNA splicing factor in the cell.

In some embodiments, detecting the presence of a functional reporter protein comprises quantifying the amount of reporter protein. In some embodiments, the reporter protein is a fluorescent or luminescent protein.

In some embodiments, the method further comprises contacting a control cell without a change-of-function or loss-of-function mutation in a recurrently mutated RNA splicing factor gene with the expression cassette and further contacting the control cell with the candidate composition.

In some embodiments, the candidate composition is selected from a small molecule, protein (e.g., antibody, or fragment or derivative thereof, enzyme, and the like), and nucleic acid construct to alter the genome or transcriptome of the cell, or a complex of a nucleic acid and protein. In some embodiments, the nucleic acid construct is an interfering RNA construct. In some embodiments, the candidate composition comprises a guide nucleic acid specific for a target sequence and an associated nuclease that modifies and/or cleaves a nucleic acid molecule upon binding of the guide nucleic acid to its target sequence. In some embodiments, the candidate composition comprises a guide nucleic acid specific for a target sequence and an associated catalytically inactive nuclease, wherein binding of the guide nucleic acid to the target sequence results in modification of transcription, splicing, or translation of the target sequence. In some embodiments, the associated nuclease is Cas9, Cas12, Cas13, Cas14, variants thereof, and the like. In some embodiments, the candidate composition comprises a Transcription Activator-Like Effector Nuclease (TALEN), Zinc Finger Nuclease (ZFN), or recombinase fusion protein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1G: Synthetic introns can mimic SF3B1 mutation-dependent mis-splicing in cancers. (1A) Workflow to identify differentially spliced events in SF3B1-mutant patient samples. (1B) Heatmap illustrating z score-normalized expression of the top-ranked, mis-spliced isoforms. Top-ranked isoforms were defined as those with |Δ(isoform expression)|>=0.1 and s.d. (isoform expression)<=0.15 across all SF3B1-mutant samples, where isoform expression is the fractional expression of each isoform and Δ(isoform expression) is defined as the absolute difference in isoform expression between each SF3B1-mutant sample and the average for samples within the same cohort lacking any splicing factor mutations. For normal tissue samples, Δ(isoform expression) defined as the absolute difference between each normal sample and all SF3B1-mutant cancer samples. Plot restricted to samples bearing the most common SF3B1K700E and R625C/H/R mutations with mutant allele expression >=25%. Samples clustered by A isoform expression of each event across all tissues and cancer types. Sample origins described in Data Availability section, below. (1C) RNA-seq read coverage plots, averaged over the indicated samples, for the six introns selected for follow-up studies. Samples from Dolatshad, H. et al. Disruption of SF3B1 results in deregulated expression and splicing of key genes and pathways in myelodysplastic syndrome hematopoietic stem and progenitor cells. Leukemia: official journal of the Leukemia Society of America, Leukemia Research Fund, UK (2014) doi:10.1038/leu.2014.331; and Tyner, J. W. et al. Functional genomic landscape of acute myeloid leukaemia. Nature 60, 277-531 (2018), incorporated herein by reference in their entireties. (1D) RT-PCR analysis of endogenous MAP3K7 and MTERFD3 splicing in cells engineered to bear the indicated mutations in endogenous SF3B1 (K562, NALM-6) or carrying them endogenously (MEL270, MEL202). MAP3K7 isoforms arise from cryptic 3′ splice site; MTERFD3 isoforms arise from both cryptic 3′ splice site usage and intron retention. n=4 (K562, NALM-6) and 2 (MEL270, MEL202) biologically independent cell lines. (1E) Schematic of the fluorescent reporter created to test synthetic intron function. (1F) Expected splicing outcomes, intron lengths, and mutation-dependent response for each tested intron. Mutation-dependent response defined as the ratio of the indicated isoforms in SF3B1-mutant:WT cells (mRNA) and median mEmerald:mCardinal signal (protein). (1G) Histograms of mEmerald:mCardinal signal, measured by flow cytometry. Arrows indicate medians (μ_{1/2}) for each genotype. Representative images from n=2 biologically independent experiments. Synthetic intron nomenclature specifies the original endogenous gene, the corresponding intron number, and synthetic intron length.

FIGS. 2A-2I: Synthetic introns enable mutation-dependent cancer cell killing. (2A) Schematic of expression construct for HSV-TK interrupted by a synthetic intron. (2B) Diagram of experiments to measure genotype-dependent differences in viability. For single-construct experiments, viability was measured directly; for screens, relative viability was inferred from relative abundance of each construct. (2C) Relative viability of K562 cells expressing the indicated constructs, measured in cells expressing each construct individually. Relative viability measured by ATP after 3 days of treatment and normalized to untreated samples. Experimental schema in (2B). Vector is hPGK-HSV-TK-P2A-mCherry. Data represented as mean±s.d. n=3 biologically independent experiments. (2D) Diagram of synMTERFD3i1-250 synthetic intron, with notable features highlighted. The illustrated sequence is set forth in SEQ ID NO:180. Splice site scores correspond to MaxEntScan (Yeo, G. & Burge, C. B. Maximum entropy modeling of short sequence motifs with applications to RNA splicing signals. Journal of computational biology: a journal of computational molecular cell biology 11, 377-394 (2004), incorporated herein by reference in its entirety) scores in HSV-TK exonic context. Lariats arising from branchpoints were identified at positions −32, −43, −48, −55, and −61 with approximate frequencies of 6%, 16%, 28%, 47%, and 3%, respectively. (2E) Diagrams of modifications in each intron relative to synMTERFD3i1-250. Deletions are specified as open intervals. The branchpoint was inserted between the indicated positions. (2F) Relative viability of K562 cells expressing the indicated constructs, measured in the mini-screen. Relative viability estimated as fold-change in representation of each construct, measured by full-length intron sequencing from genomic DNA, at day 6 for GCV-treated relative to untreated samples. GCV concentration, 100 ug/mL. Vector is hPGK-PuroR-P2A-HSV-TK. Data represented as mean±s.d. Standard deviation was estimated as sample proportion standard deviation. (2G) Relative viability of K562 cells expressing the indicated constructs, measured in cells expressing each construct individually. Viability estimates from these single-construct experiments are concordant with estimates from parallelized screening in (2F); note that fold-changes are greater in this experiment because of its longer duration (11 vs. 6 days). Relative viability measured by ATP after 11 days of treatment and normalized to PBS-treated samples. GCV concentration, 100 ug/mL. Vector is hPGK-PuroR-P2A-HSV-TK. Data represented as mean±s.d. n=3 biologically independent experiments. (2H) As (2G), but for breast epithelial (MCF10A) cells with or without an SF3B1 mutation at the endogenous locus. (2I) RT-PCR demonstrating mutation-dependent excision of the synthetic intron in the experiments from (2G) and (2H).

FIGS. 3A-3I: Massively parallel screening reveals critical elements governing synthetic intron function. All modifications are diagrammed and described in detail in the TABLE 2. (3A) Relative fold-change of synthetic introns derived from synMTERFD3i1-250 by deletion of 100 consecutive nt. Each horizontal line indicates the nucleotides deleted in a single such variant, with the vertical position indicating its fold-change in SF3B1-mutant (SF3B1^{K700E/+}) or WT (SF3B^{+/+}) cells. Plot restricted to introns with log 2 (fold-change)<−0.5 or >−0.1 in SF3B1-mutant and WT cells, respectively. Dashed lines, deletion used to create synMTERFD3i1-150 (first 25 nt and last 125 nt preserved). (3B) As (3A), but for longer deletions, resulting in introns of lengths 125, 100, 85 or 75 nt. Deletions were made to synMTERFD3i1-150 or synMTERFD3i1-100. Dashed lines, deletion used to create synMTERFD3i1-100 (first 15 nt and last 85 nt preserved). Shading designations same as in (3A). (3C) Box plots illustrating relative fold-changes for all introns derived from synMTERFD3i1-150 by single-nucleotide mutations, where the mutations were within the 10 5′-most nucleotides (5′ss), 26 3′-most nucleotides (any 3′ss), or neither region (neither). Colors as in (3A). (3D) Box plots illustrating relative fold-changes for all introns derived from synMTERFD3i1-150 by one or two single-nucleotide mutations, grouped by the difference in splice site strengths (computed by MaxEntScan (Yeo, G. & Burge, C. B. Maximum entropy modeling of short sequence motifs with applications to RNA splicing signals. Journal of computational biology: a journal of computational molecular cell biology 11, 377-394 (2004)) between the most intron-distal and most intron-proximal 3′ splice sites. Shading designations as in (3A). (3E) Top, histogram illustrating the distribution of relative fold-changes for introns created by randomly shuffling the nucleotides between +10 of the 5′ss and −30 of the canonical 3′ss of synMTERFD3i1-150. Bottom, corresponding histogram for introns created by introducing single-nucleotide mutations within the same region to synMTERFD3i1-150. Arrows, relative fold-changes for unmutated synMTERFD3i1-150 in SF3B1-mutant (left arrow) or WT (right arrow) cells. Shading designations as in (3A). (3F) Top, purine:pyrimidine ratio calculated with a 5 nt sliding window. The illustrated sequence is set forth as SEQ ID NO:181. Bottom, schematic of synMTERFD3i1-150 intron, with key features highlighted. (3G) Line plots illustrating relative fold-changes for introns with the indicated perturbations at the indicated positions to synMTERFD3i1-150 for SF3B1-mutant (SF3B1K700E/+) or WT (SF3B′+i+) cells. Data presented as geometric mean of fold-changes for the three closest introns (line)±geometric s.d. from the mean (shading). From top to bottom, perturbations are: sliding deletions (5 nt); sliding conversion of 4 nt to a 3′ss (CAGG), where position corresponds to placement of the first G in CAGG; sliding conversion of all Ys within 6 nt to G, where position corresponds to center of the 6 nt window; sliding insertion of consensus branchpoint context (tactaAca), where position represents point of insertion; simultaneous ablation of all four commonly used branchpoints (A>G) and sliding insertion of consensus branchpoint context (tactaAca), where position represents point of insertion. (3H) Sequence logo plots illustrating relative fold-change for single-nucleotide mutations to synMTERFD3i1-150 in WT (top) or SF3B1-mutant (bottom) cells. Height of each nucleotide indicates the fold-change for that mutation. (3I) Arc plot illustrating the directions and magnitudes of synergistic fold-changes for each pair of mutations at the 5′ss or 3′ss to synMTERFD3i1-150 in WT (top) or SF3B1-mutant (bottom) cells. Vertical height indicates fold-change from combinatorial mutation relative to that expected from the two underlying single-nucleotide mutations. Plot restricted to |log 2 (synergy)|>1.

FIGS. 4A-4I: Synthetic introns enable mutation-dependent cancer cell targeting in vivo. (4A) Schematic of xenograft experiments with K562 cells expressing Luciferase and HSV-TK interrupted by synMTERFD3i1-150. K562 cells were intravenously injected into sub-lethally irradiated (250 cGy) NOD-scid IL2rgnull (NSG) mice (2M cells/mouse). n=10 (WT) and 9 (SF3B1-mutant) mice per cohort. (4B) Quantification of tumor burden, estimated by whole-body bioluminescent signal. Each point represents a single mouse. (4C) Representative bioluminescence images from cohorts described in (4A). (4D) Survival of mice from cohort described in (4A). p computed with logrank test. Comparisons not illustrated on plot are not significant (p_{+/+ GCV vs. PBS}=0.534; p_{K700E/+PBS vs.+/+PBS}=0.823). The cause of death for the one GCV-treated animal engrafted with an SF3B1-mutant leukemia which died by experiment endpoint was unclear, as this animal had minimal leukemic burden by imaging or necropsy. (4E) Survival curves of NSG mice engrafted with SF3B1 WT (top) or SF3B1 K700E (bottom) MOLM-13 cells expressing Luciferase and HSV-TK interrupted by synMTERFD3i1-150 followed by treatment with either PBS or GCV. p_{K700E/+ PBS vs. pK700E/+GCV}=0.011. n=10 mice/group. (4F) Representative bioluminescence images from cohorts described in (4E). (4G) Tumor volumes of mice engrafted with SF3B1 WT or SF3B1 K700E T47D cells expressing HSV-TK interrupted by synMTERFD3i1-150 followed by treatment with either PBS or GCV. T47D cells were engrafted subcutaneously. Data represented as mean±s.d. n=8 mice/group. (4H) Tumor volumes of mice engrafted with SF3B1 WT MEL285 cells (left) or SF3B1 R625G MEL202 cells (right) expressing HSV-TK interrupted by synthetic intron 6700 (synMTERFD3i1-150 with A>C at −7 nt; A>C at −19 nt) followed by treatment with either PBS or GCV. All cells were engrafted subcutaneously. Data represented as mean±s.d. n=10 mice/group. (4I) Representative gross images of the tumors from (4H) at day 27 post-implantation.

FIGS. 5A-5C: Delivery of synthetic intron-containing constructs to established tumors in vivo is feasible. (5A) Tumor volumes of mice engrafted with MEL202 cells, which bear an endogenous SF3B1R625G mutation. HSV-TK interrupted by synMTERFD3i1-150 with A>C at −7 nt; A>C at −19 nt was delivered via direct intratumoral lentiviral injection at the indicated time points. (5B) Representative images from experiment in (5A). (5C) RT-PCR demonstrating expression of spliced HSV-TK in tumors weeks after the last lentiviral injection.

FIGS. 6A-6I: Validation of SF3B1 mutation-dependent differential splicing for endogenous and synthetic introns. (6A) As FIG. 1B, but additionally illustrating splicing patterns for normal bone marrow (n=3) and cancer samples lacking SF3B1 mutations from each studied cohort. n=3 randomly chosen SF3B1-WT samples illustrated for each cohort, with the exceptions of uveal melanoma, acute myeloid leukemia, and MDS, for which additional samples were illustrated given the high frequency of SF3B1 mutations in these disorders. (6B) As FIG. 1B, but additionally including all samples with SF3B1K666E/N/R/T mutations with mutant allele expression >=25%. (6C) RT-PCR analysis of competing 3′ splice site (3′ss) usage within endogenous introns of ORAI2 and TMEM14C in K562 cells engineered to bear the indicated mutations in endogenous SF3B1. n=4 biologically independent cell lines. (6D) As (6C), but for intron retention within endogenous introns of MYO15B and SYTL1. (6E) RT-PCR analysis of endogenous MAP3K7 and MTERFD3 splicing in primary samples from patients with acute myeloid leukemia (AML) and myelodysplastic syndromes (MDS), as well as pancreatic ductal adenocarcinoma (PDAC) cell lines that are wild-type (WT) or mutant for SF3B1 K700E and K666R/N/M/T mutations. (6F) Sanger sequencing illustrating three distinct MTERFD3 isoforms arising from three competing 3′ss (two cryptic 3′ss and one canonical, frame-preserving, 3′ss). The three lower bands for the MTERFD3 RT-PCR illustrated in FIG. 1D, were isolated, cloned, and sequenced to identify the specific 3′ss that were used for splicing of each isoform. (6G) RT-PCR analysis of synthetic intron splicing for the indicated introns following transfection of the fluorescent reporter construct into isogenic K562 cells with the indicated SF3B1 genotypes. n=2 biologically independent cell lines and n=3 biologically independent experiments. (6H) As (6G), but for the indicated introns. n=2 biologically independent cell lines. (6I) Schematic of split HSV-TK construct with mCherry.

FIGS. 7A-7F: Hallmark SF3B1 mutation-responsive events are specific to SF3B1 mutations and recapitulated in breast epithelial cells. (7A) RNA-seq read coverage plot for K562 cells (top) and MCF10A cells (bottom) engineered to have the illustrated genotypes, illustrating specificity of mutant SF3B1-dependent usage of an intron-proximal cryptic 3′ss in MAP3K7. Each indicated mutant allele is present as a single copy in the endogenous locus in otherwise WT cells. Neither SRSF2 nor U2AF1 mutations induce the splicing changes caused by SF3B1 mutations. These RNA-seq data complement the related RT-PCR studies in FIGS. 1D and 6E-6G. (7B) As (7A), but for mutant SF3B1-dependent mis-splicing in MTERFD3. The MTERFD3 intron contains two specific splicing changes in SF3B1-mutant cells: increased intron excision (left) and increased usage of an intron-distal competing 3′ss (right). (7C) Top, RT-PCR demonstrating mutation-dependent excision of the synthetic intron in T47D cells expressing doxycycline-inducible WT or mutant (K700E) SF3B1. Bottom, relative viability of cells illustrated above following treatment with ganciclovir (GCV). Data represented as mean±s.d. (7D) As (7C), but for MOLM-13 cells expressing doxycycline-inducible WT or mutant (K700E) SF3B1. (7E) As (7C), but for Panc05.04 cells, which bear the endogenous mutation SF3B1Q699H/K700E. (7F) Relative viability of U2AF1^{S34F/+} or SRSF2^{P95H/+} K562 knockin cells expressing HSV-TK interrupted by synMTERFD3i1-150 or negative and positive control constructs. Data represented as mean±s.d.

FIGS. 8A-8H: Massively parallel screening reveals critical elements governing the function of very short synthetic introns. (8A) As FIG. 3B, but restricted to deletions resulting in an intron of length 100 nt. (8B) As FIG. 3C, but for mutations to synMTERFD3i1-100. (8C) As FIG. 3D, but for mutations to synMTERFD3i1-100. (8D) As FIG. 3E, but for mutations to synMTERFD3i1-100. (8E) As FIG. 3F, but illustrates synMTERFD3i1-100. The illustrated sequence is set forth in SEQ ID NO:182. (8F) As FIG. 3G, but for mutations to synMTERFD3i1-100. (8G) As FIG. 3H, but for mutations to synMTERFD3i1-100. (8H) Box plot illustrating relative fold-changes for introns derived by inserting a very strong 3′ss and key upstream sequence elements (1-4 consensus branchpoints, inserted at positions+25 to +50 relative to the 5′ss, and TTTTTTTTTTTTTTTTTCAG (SEQ ID NO:72), representing a long polypyrimidine tract immediately followed by a 3′ss) within synMTERFD3i1-100, with 0-8 nt between the last nucleotide of the inserted TTTTTTTTTTTTTTTTTCAG (SEQ ID NO:72) and the canonical 3′ss.

FIGS. 9A-9H: Branchpoint manipulation and combinatorial 3′ss mutations enhance SF3B1 mutation-dependent splicing. (9A) Diagrams of modifications in each intron relative to synMTERFD3i1-150. Deletions are specified as open intervals. Branchpoints were inserted in between the indicated positions. (9B) Relative viability of K562 cells expressing the indicated constructs, measured in the full screen. Relative viability estimated as fold-change in representation of each construct, measured by full-length intron sequencing from genomic DNA, at day 8 for GCV-treated relative to untreated samples. GCV concentration, 100 ug/mL. Vector is hPGK-PuroR-P2A-HSV-TK. Data represented as mean±s.d. Standard deviation was estimated as sample proportion standard deviation. (9C) Relative viability of K562 cells expressing the indicated constructs, measured in cells expressing each construct individually. Viability estimates from these single-construct experiments are concordant with estimates from parallelized screening in (9B); note that fold-changes are greater in this experiment because of its longer duration (11 vs. 8 days). Relative viability measured by ATP after 11 days of treatment and normalized to PBS-treated samples. GCV concentration, 100 ug/mL. Vector is hPGK-PuroR-P2A-HSV-TK. Data represented as mean±s.d. n=3 biologically independent experiments. (9D) RT-PCR demonstrating mutation-dependent excision of the synthetic intron in the experiments from (9C). Constructs are ordered from left to right in the same order as from top to bottom in (9B). (9E) RT-PCR demonstrating mutation-dependent excision of synMTERFD3i1-150 or control synthetic introns in K562 cells with or without knockin of the SF3B1 K700E or K666N mutations or MEL202 cells with an endogenous SF3B1 R625G mutation. (9F) RT-PCR of a series of synthetic introns in MEL202 cells (SF3B1R625G). (9G) RT-PCR of HSV-TK interrupted by synthetic intron 6700 (synMTERFD3i1-150 with A>C at −7 nt; A>C at −19 nt) in uveal melanoma cell lines wild-type or mutant for SF3B1. (9H) Relative viability of the cells in (9G) following treatment with ganciclovir (GCV). Data represented as mean±s.d.

FIGS. 10A-10F: Synthetic introns enable mutation-dependent cancer cell targeting in vivo. (10A) Schematic of xenograft experiments with MOLM-13 cells expressing doxycycline-inducible SF3B1 (wild-type or K700E), luciferase, and HSV-TK interrupted by synMTERFD3i1-150. MOLM-13 cells were intravenously injected into sub-lethally irradiated (250 cGy) NOD-scid IL2rgnull (NSG) mice (2M cells/mouse). Doxycycline was provided in feed on day 1 and intraperitoneal GCV or PBS was administered at day 11 three times/week. (10B) Radiance of experiment in (10A). Each point represents an individual animal. *p<0.05. (10C) Schematic of xenograft experiments with T47D cells expressing doxycycline-inducible SF3B1 (wild-type or K700E), luciferase, and HSV-TK interrupted by synMTERFD3i1-150. T47D cells were injected subcutaneously into NSG mice (2M cells/mouse). Estrogen was provided via a subcutaneous estrogen implant and intraperitoneal GCV or PBS was administered at day 7 three times/week. (10D) Tumor volume at day 20 of experiment in (10C). Data represented as mean±s.d. (10E) Kaplan-Meier curves of NSG mice subcutaneously engrafted with MEL285 (left) or MEL202 (right) cells expressing HSV-TK interrupted by synthetic intron 6700 (synMTERFD3i1-150 with A>C at −7 nt; A>C at −19 nt) following PBS or GCV treatment. (10F) Tumor volumes of mice from (10E) at day 27. Data represented as mean±s.d.

FIGS. 11A-11F: AAV-mediated delivery of IL-2 constructs. (11A) Schematic of AAV transfer plasmid with IL-2 interrupted by the synMTERFD3i1-150 synthetic intron. This transfer plasmid was used for AAV2-mediated delivery of the illustrated IL-2 construct (2,000 vg/cell). (11B) RT-PCR illustrating SF3B1 mutation-dependent splicing of the construct in (11A) following AAV2-mediated delivery to the indicated cells. (11C) Bar plot illustrating results of ELISA assay for IL-2 following AAV2-mediated delivery of a negative control (AAV-GFP) or the construct shown in (11A) (AAV-IL-2-synMTERFD3i1-150). (I1D) Schematic of AAV transfer plasmid with HSV-TK interrupted by the synMTERFD3i1-150 synthetic intron, followed by P2A+IL-2. This transfer plasmid was used for AAV2-mediated delivery of the illustrated HSV-TK+IL-2 construct (2,000 vg/cell). (11E) RT-PCR illustrating SF3B1 mutation-dependent splicing of the construct in (11D) following AAV2-mediated delivery to the indicated cells. (11F) Bar plot illustrating results of ELISA assay for IL-2 following AAV2-mediated delivery of the construct shown in (11D).

FIGS. 12A-12C: Exemplary fluorescent reporters with synthetic introns. (12A) Schematic of fluorescent reporter with Emerald interrupted by the synMTERFD3i1-150 synthetic intron. mCardinal is a positive control signal. (12B) Flow cytometry density plots illustrating the ratio of mEmerald to mCardinal following delivery of the reporter in (12A) to the indicated cells. (12C) As (12B), but for the indicated cells.

### DETAILED DESCRIPTION

Many cancers carry recurrent mutations in RNA splicing factor genes, or “spliceosomal mutations,” which induce sequence-specific changes in RNA splicing. In this usage, “cancer” may refer to any dysplastic disease, neoplastic disease, or other disease characterized by disordered cell differentiation, insufficient cell production, impaired cell death, or accelerated cell proliferation. These diseases include solid tumors, malignant ascites, myelodysplastic syndromes, leukemias, lymphomas, and other malignancies and disorders of the bone marrow and hematopoietic system, bone marrow failure syndromes, connective tissue malignancies, metastatic disease, minimal residual disease following transplantation of organs or stem cells, multi-drug resistant cancers, primary or secondary malignancies, angiogenesis related to malignancy, or other forms of cancer. For example, SF3B1 is the most commonly mutated splicing factor gene. SF3B1 mutations occur in many cancers, including myelodysplastic syndromes (MDS), chronic lymphocytic leukemia (CLL), uveal melanoma, mucosal melanoma, skin melanoma, breast cancer, pancreatic cancer, and others. The inventors have previously demonstrated that SF3B1 and other common splicing factor mutations cause highly specific changes in RNA splicing mechanisms, such that cancer cells carrying mutations in SF3B1 or other RNA splicing factors do or do not efficiently remove introns with particular sequences.

This disclosure is based on an investigation to develop anti-tumor therapies that harness this change in RNA splicing activity to drive spliceosomal mutation-dependent gene expression in cancers and selectively eliminate these tumors. As described in more detail below, synthetic “designer” introns were engineered that were spliced differently in leukemia, melanoma, and breast cancer cells bearing the most common SF3B1 mutations relative to their splicing patterns in wild-type cells—for example, introns that were efficiently spliced out of SF3B1-mutant cells in their entirety, but not efficiently spliced or incompletely spliced out of wild-type cells; or introns that were partially spliced out of SF3B1-mutant leukemia, melanoma, and breast cancer cells, leaving a portion of the intron in the mature transcript, but fully spliced out of wild-type cells—to yield mutation-dependent protein production. A massively parallel screen of over 8,000 distinct introns delineated ideal intronic size, mapped essential sequence elements, and revealed the basis of mutation-dependent splicing. Key synthetic introns from the screen enabled SF3B1 mutation-dependent delivery of herpes simplex virus thymidine kinase (HSV-TK) and subsequent ganciclovir-mediated elimination of leukemia, melanoma, and breast cancer cells bearing SF3B1 mutations in vitro and in vivo, while leaving wild-type cells unaffected. This approach significantly decreased the growth of otherwise lethal leukemia and melanoma xenografts. Key synthetic introns also enabled SF3B1 mutation-dependent expression of IL-2 in cancer cells, as well as SF3B1 mutation-dependent simultaneous expression of HSV-TK and IL-2 in cancer cells, in both cases with the construct delivered by adeno-associated virus (AAV). The modular, compact, and specific nature of synthetic introns thereby provide a means to exploit cancer-specific changes in RNA splicing for genotype-dependent gene expression and gene therapy. Additionally, this understanding of sequence parameters driving altered RNA splicing activity in these cells allows creation of constructs that selectively express proteins in cells either with or without defined spliceosomal mutations in order to identify compounds that suppress mutant splicing factor activity and restore normal splicing.

Artificial Nucleic Acid Intron Construct

In accordance with the forgoing, in one aspect the disclosure provides an artificial nucleic acid intron construct. The artificial nucleic acid intron construct comprises an intron sequence, hereafter referred to as artificial intron, intron sequence, intron domain, or simply intron. The term “artificial” refers to the sequence of the construct (e.g., including the intron sequence), which does not occur in nature, but has been newly created or derived from a naturally occurring sequence. As used in this context, the term “derived” indicates that the resulting construct sequence has been engineered and contains structural (e.g., sequence) alterations from the naturally occurring sequence. As explained in more detail in the Examples, the inventors have determined several features that can be leveraged to modify the susceptibility for splicing in cells characterized by a mutation in an RNA splicing factor gene, which permits selective splicing, selective inhibition of splicing, or selective modification of splicing of the intron from the context sequence (e.g., surrounding exonic sequences), compared to cells that lack the mutation in the RNA splicing factor gene.

In some embodiments, the intron or intron domain comprises at least the following features:
a 5′ splice site;
a canonical 3′ splice site;
at least one cryptic 3′ splice site;
a pyrimidine-rich domain comprising at least 6 consecutive nucleotides; and
at least one branchpoint at least 15 nucleotides upstream of the canonical 3′ splice site.

Addressing the 5′ splice site, the disclosed artificial intron can comprise any functional 5′ splice site sequence that is typically recognized by splicing factors. 5′ splice sites are known in the art and are encompassed by the present disclosure. Exemplary, non-limiting 5′ splice sites encompassed by the present disclosure comprise a sequence selected from GTGAG, GTAAG, GTGCG, GTACG, GTGGG, GTAGG, GTGTG, GTATG, and GTATC. As would be evident to a person of ordinary skill in the art, the 5′ splice site is by definition positioned upstream, or 5′ to, the other recited elements of the intron sequence.

The term “canonical” 3′ splice site refers to a splice site whose usage results in preservation of the open reading frame if the intron is inserted into a coding DNA sequence and subsequently spliced, such that no in-frame termination codons are introduced into the coding sequence if the canonical 3′ splice site is used during the splicing process. For example, a canonical 3′ splice site may lie at the 3′ end of an intron, such that insertion of this intron into a coding sequence and subsequent usage of the canonical 3′ splice site during splicing results in complete excision of the intron from the mature RNA transcript, thereby preserving the open reading frame. The term “cryptic” 3′ splice site refers to a splice site whose usage results in disruption of the open reading frame if the intron is inserted into a coding DNA sequence and subsequently spliced, such that one or more in-frame termination codons are introduced into the coding sequence if the cryptic 3′ splice site is used during the splicing process. For example, a cryptic 3′ splice site may lie upstream, or 5′ to, the canonical 3′ splice site, such that insertion of this intron into a coding sequence and subsequent usage of the cryptic 3′ splice site during splicing does not result in complete excision of the intron from the mature RNA transcript, thereby disrupting the open reading frame.

Canonical 3′ splice sites are known in the art, which are encompassed by the present disclosure. Exemplary, non-limiting, canonical 3′ splice sites encompassed by the present disclosure comprise at least a core sequence of AAG, CAG, GAG, and TAG. The 3′ splice sites can be longer, however, such as selected from the non-limiting list including AACAG, AATAG, ACCAG, ACTAG, ATCAG, ATTAG, AGCAG, AGTAG, CACAG, CATAG, CCCAG, CCTAG, CTCAG, CTTAG, CGCAG, CGTAG, TACAG, TATAG, TCCAG, TCTAG, TTCAG, TTTAG, TGCAG, TGTAG, GACAG, GATAG, GCCAG, GCTAG, GTCAG, GTTAG, GGCAG, and GGTAG, all of which are encompassed by the present disclosure. Exemplary, non-limiting cryptic 3′ splice sites can comprise a sequence selected from AAG, CAG, GAG, TAG, ATG, CTG, GTG, and TTG.

The at least one cryptic 3′ splice site is positioned within about 100 nucleotides (e.g., including within about 90, 80, 70, 60, 50, 40, 30, 20, 10 nucleotides or any range therein) upstream of the canonical 3′ splice site or within about 50 nucleotides (e.g., including within about 40, 30, 20, 10 nucleotides, or any range therein) downstream of the canonical 3′ splice site. As used herein, the term “upstream” refers to a position in a nucleic acid molecule or sequence that is on the 5′ side of the reference position within the nucleic acid molecule or sequence. Conversely, the term “downstream” refers to a position in a nucleic acid molecule or sequence that is on the 3′ side of the reference position within the nucleic acid molecule or sequence.

The artificial intron can comprise a plurality (e.g., 2, 3, 4, 5, 6, 7, 8, 9, 10, or more) of cryptic 3′ splice sites, which can be the same or different from each other. For example, each of the plurality of the cryptic 3′ splice sites can comprise a sequence independently selected from AAG, CAG, GAG, TAG, ATG, CTG, GTG, and TTG. In some embodiments, the intron comprises a plurality of cryptic 3′ splice sites within about 100 nucleotides (e.g., including within about 90, 80, 70, 60, 50, 40, 30, 20, 10 nucleotides or any range therein) upstream of the 3′ canonical splice site. In some embodiments, the intron comprises a plurality of cryptic 3′ splice sites within about 100 nucleotides (e.g., including within about 90, 80, 70, 60, 50, 40, 30, 20, 10 nucleotides or any range therein) downstream of the 3′ canonical splice site. In some embodiments, the intron comprises one or more cryptic 3′ splice sites within about 100 nucleotides (e.g., including within about 90, 80, 70, 60, 50, 40, 30, 20, 10 nucleotides or any range therein) upstream of the 3′ canonical splice site and one or more cryptic 3′ splice sites within about 100 nucleotides (e.g., including within about 90, 80, 70, 60, 50, 40, 30, 20, 10 nucleotides or any range therein) downstream of the 3′ canonical splice site.

The sequence of the pyrimidine-rich domain is contiguous sequence that is at least about 60% pyrimidine nucleotides (e.g., including at least about 60%, 65%, 70%, 75%, 80%, 85%, 90%, and 95% pyrimidine nucleotides). Examples of pyrimidine nucleotides are cytosine, thymine, and uracil, although the disclosure also encompasses non-canonical derivatives and analogs thereof that preserve the pyrimidine core structure. The pyrimidine-rich domain is positioned within at least about 50 nucleotides of a cryptic 3′ splice site (e.g., including within about 40, 30, 20, 10 nucleotides, or any range therein). As used in this context, the reference to the distance, or range of distances, between the pyrimidine-rich domain and the cryptic 3′ splice site refers to the number of intervening nucleotides between and including the closest nucleotide of the pyrimidine-rich domain and the splice site. Thus, a portion of the pyrimidine-rich domain (including a substantial portion) can be outside the indicated range. The pyrimidine-rich domain can be located upstream or downstream of the cryptic 3′ splice site.

In some embodiments, the pyrimidine-rich domain comprises at least 15 consecutive nucleotides, such as about 15, 20, 25, 30, 35, 40, 45, 50, or more nucleotides. In some embodiments, the pyrimidine-rich domain has a sequence with at least 60% pyrimidine nucleotides (e.g., including at least about 60% 65%, 70%, 75%, 80%, 85%, 90%, and 95% pyrimidine nucleotides) and is also at least 40% thymine nucleotides (e.g., including at least about 45%, 50%, 55%, 60% 65%, 70%, 75%, 80%, 85%, and 90% thymine nucleotides), which contribute to the pyrimidine proportion indicated above. In some embodiments, the pyrimidine-rich domain is within at least 30 nucleotides (e.g., including within about 25, 20, 10 nucleotides, or any range therein) of a cryptic 3′ splice site. As indicated above, the expression of proximity to the splice site refers to the number of intervening nucleotides between and including the closest nucleotide of the pyrimidine-rich domain and the splice site. In some embodiments, the pyrimidine-rich domain comprises a sequence with at least 50% sequence identity (e.g., including at least about 55%, 60% 65%, 70%, 75%, 80%, 85%, 90%, and 95% sequence identity) to any 20 or more contiguous nucleotides selected from the sequence CATTTCTATGTTTTATTTTACTTTGTCTTTATCCT (SEQ ID NO:49). In some embodiments, the pyrimidine-rich domain comprises two, three, four, or more of the elements described in this paragraph, in any combination.

As indicated above, the intron comprises at least one branchpoint at least 15 nucleotides upstream of the canonical 3′ splice site. A branchpoint is a nucleotide that participates in a specific step during splicing catalysis. RNA splicing generally proceeds via a two-step process defined by sequential transesterification reactions between three nucleotides: the first nucleotide of the 5′ splice site, the branch nucleotide (branchpoint) upstream of the 3′ splice site, and the last nucleotide of the 3′ splice site. In the first step of splicing, the 2′ OH group of the branchpoint engages in a nucleophilic attack on the phosphate between the upstream exon and the 5′ splice site, forming a 2′-5′ phosphodiester linkage (the “branch”) characteristic of the lariat RNA intermediate and releasing the upstream exon. The 3′ OH group of the now-free upstream exon then engages in a nucleophilic attack on the phosphate between the 3′ splice site and the downstream exon, resulting in release of the intronic lariat and exon ligation (for review, see Wahl et al., The Spliceosome: Design Principles of a Dynamic RNP Machine, Cell, 2009, 136(4):701-718, incorporated herein by reference in its entirety). The intronic lariat is then linearized via debranching and subsequently degraded. In some embodiments, the at least one branchpoint is at least 20 nucleotides upstream of the canonical 3′ splice site, and wherein the branchpoint nucleotide is an adenine. In some embodiments, the branchpoint and surrounding sequence context has sequence identity of at least 50% to the sequence tactaAca, where the nucleotide represented by the uppercase A is the branchpoint nucleotide and is preserved in the sequence. Other branchpoint nucleotides and surrounding sequence contexts are known in the art and are encompassed by the present disclosure.

Intron lengths can vary widely in natural settings and still be functionally spliced to result in a contiguous coding sequence in mature RNA transcripts. For example, typical intron lengths in the human genome can be approximately 6,400 nucleotides. Accordingly, the disclosed intron is not limited by length. In some embodiments, the intron is at least about 50 nucleotides to about 1500 nucleotides, such as at least about 50 nucleotides to about 1250 nucleotides, about 50 nucleotides to about 1000 nucleotides, about 50 nucleotides to about 900 nucleotides, about 50 nucleotides to about 800 nucleotides, about 50 nucleotides to about 700 nucleotides, about 50 nucleotides to about 600 nucleotides, about 50 nucleotides to about 500 nucleotides, about 100 nucleotides to about 1500 nucleotides, about 100 nucleotides to about 1250 nucleotides, about 100 nucleotides to about 1000 nucleotides, about 100 nucleotides to about 900 nucleotides, about 100 nucleotides to about 800 nucleotides, about 100 nucleotides to about 700 nucleotides, about 100 nucleotides to about 600 nucleotides, about 100 nucleotides to about 500 nucleotides, and any length or range therein.

The intron can be derived from a naturally occurring intron from any eukaryotic organism (referred to as a “source” intron). As indicated above, the term “derived from” refers to the retention of certain structural features of the source intron, but wherein the artificial intron also has certain variations that deviate from the source intron. In some embodiments, a sequence “derived from” a source can comprise a sequence or subsequence (i.e., subdomain) is about 30%, 35%, 40%, 45%, 50%, 55%, 60%, 65%, 70%, 75%, 80%, 85%, 90%, 95%, 98%, or 99% identical to the source sequence or subsequence (i.e., subdomain), as determined by standard methods. The subdomain can be, e.g., at least about 20, 30, 40, 50, 60, 70, 80, 90, 100, 125, 150, 175, 200, 225, 250, 275, 300, 325, 350, 375, 300, or more contiguous nucleotides of the overall sequence. As a non-limiting example, the intron can be derived from a human wildtype intron. Examples of such human source introns from which the disclosed intron can be derived include intron 1 of MTERFD3, intron 4 of MYO15B, intron 10 of SYTL1, intron 11 of SYTL1, intron 4 of MAP3K7, intron 1 of ORAI2, and intron 1 of TMEM14C, although the disclosed intron can be derived from others as well. To illustrate, in some embodiments, the source intron is selected from one of the following: intron 1 of MTERFD3 comprising a sequence set forth in SEQ ID NO:2; intron 4 of MYO15B comprising a sequence set forth in SEQ ID NO:8; intron 10 of SYTL1 comprising a sequence set forth in SEQ ID NO:13; intron 11 of SYTL1 comprising a sequence set forth in SEQ ID NO: 15; intron 4 of MAP3K7 comprising a sequence set forth in SEQ ID NO:22; intron 1 of ORAI2 comprising a sequence set forth in SEQ ID NO:26; and intron 1 of TMEM14C comprising a sequence set forth in SEQ ID NO:30.

The exemplary source introns indicated above are, in exemplary wild-type contexts, flanked by upstream and downstream exon sequences as follows: intron 1 of MTERFD3 (comprising a sequence set forth in SEQ ID NO:2) is flanked by exon 1 (SEQ ID NO:1) and exon 2 (SEQ ID NO:3) of MTERFD3; intron 4 of MYO15B (comprising a sequence set forth in SEQ ID NO:8) is flanked by exon 4 (SEQ ID NO:7) and exon 5 (SEQ ID NO:9) of MYO15B; intron 10 of SYTL1 (comprising a sequence set forth in SEQ ID NO:13) is flanked by exon 10 (SEQ ID NO:12) and exon 11 (SEQ ID NO:14) of SYTL1; intron 11 of SYTL1 (comprising a sequence set forth in SEQ ID NO:15) is flanked by exon 11 (SEQ ID NO:14) and exon 12 (SEQ ID NO:16) of SYTL1; intron 4 of MAP3K7 (comprising a sequence set forth in SEQ ID NO:22) is flanked by exon 4 (SEQ ID NO:21) and exon 5 (SEQ ID NO:23) of MAP3K7; intron 1 of ORAI2 (comprising a sequence set forth in SEQ ID NO:26) is flanked by exon 1 (SEQ ID NO:25) and exon 2 (SEQ ID NO:27) of ORAI2; and intron 1 of TMEM14C (comprising a sequence set forth in SEQ ID NO:30) is flanked by exon 1 (SEQ ID NO:29) and exon 2 (SEQ ID NO:31) of TMEM14C.

The disclosed intron can be obtained, in part, by removing an interior portion from the source intron sequence. Accordingly, in some embodiments, the disclosed intron has a higher sequence similarity to 5′ end and 3′ end domains of the source intron sequence compared to an interior domain of the source sequence. For example, the 5′ end domain and/or 3′ end domain can have a minimal sequence identity to a corresponding 5′ end and/or 3′ end domain of the source intron sequence of at least approximately 25% or 30%, and lack any discernable identity or similarity to an interior domain of the source intron sequence. In some embodiments, the disclosed intron has a 5′ end domain with a length of about 10 to about 150 nucleotides (e.g., about 10, 11, 12, 13, 14, 15, 16, 17, 18, 19, 20, 25, 30, 35, 40, 45, 50, 55, 60, 65, 70, 80, 85, 90, 95, 100, 105, 110, 115, 120, 125, 130, 135, 140, 145, or 150 nucleotides), wherein the sequence has at least about 30% sequence identity (e.g., at least about 30%, 40%, 50%, 60%, 70%, 80%, or 90% sequence identity) to a corresponding sequence of the 5′-most 10 to about 150 nucleotides of the wildtype intron. Exemplary wildtype source intron sequences are indicated above. In some embodiments, the disclosed intron has a 3′ end domain with about 50 to about 350 nucleotides (e.g., about 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, or 350 nucleotides) having at least 30% sequence identity (e.g., at least about 30%, 40%, 50%, 60%, 70%, 80%, or 90% sequence identity) to a corresponding sequence of the 3′-most 50 to about 350 nucleotides of the wildtype intron. In one embodiment, the disclosed intron has a 5′ end domain with a length of about 15-30 nucleotides (e.g., about 15, 20, 25, or 30 nucleotides) wherein the sequence has at least about 30% sequence identity (e.g., at least about 30%, 40%, 50%, 60%, 70%, 80%, or 90% sequence identity) to a corresponding sequence of a 15-30 nucleotide portion (e.g., the 5′-most 15 to about 30 nucleotides) of the wildtype intron and a 3′ end domain with about 80 to about 130 nucleotides (e.g., about 80, 85, 90, 95, 100, 105, 110, 115, 120, 125, 130 nucleotides) having at least 30% sequence identity (e.g., at least about 30%, 40%, 50%, 60%, 70%, 80%, or 90% sequence identity) to a corresponding sequence of a 80-130 nucleotide portion (e.g., the 3′-most 80 to about 130 nucleotides) of the wildtype intron. In further embodiments, the disclosed intron has a 5′ end domain with a length of about 25 nucleotides (e.g., about 20-30 nucleotides) wherein the sequence has at least about 30% sequence identity (e.g., at least about 30%, 40%, 50%, 60%, 70%, 80%, or 90% sequence identity) to a corresponding sequence of a 25 nucleotide portion (e.g., the 5′-most 20 to about 30 nucleotides) of the wildtype intron and a 3′ end domain with about 80 to about 130 nucleotides (e.g., about 80, 85, 90, 95, 100, 105, 110, 115, 120, 125, 130 nucleotides) having at least 30% sequence identity (e.g., at least about 30%, 40%, 50%, 60%, 70%, 80%, or 90% sequence identity) to a corresponding sequence of 80-130 nucleotides (e.g., the 3′-most 80 to about 130 nucleotides) of the wildtype intron. In some embodiments, the disclosed intron has a 5′ end domain with a length of about 15 nucleotides wherein the sequence has at least about 30% sequence identity (e.g., at least about 30%, 40%, 50%, 60%, 70%, 80%, or 90% sequence identity) to a corresponding sequence of a 15 nucleotide portion (e.g., the 5′-most 15 nucleotides) of the wildtype intron and a 3′ end domain with about 85 nucleotides having at least 30% sequence identity (e.g., at least about 30%, 40%, 50%, 60%, 70%, 80%, or 90% sequence identity) to a corresponding sequence of 85 nucleotides (e.g., the 3′-most 85 nucleotides) of the wildtype intron.

In some embodiments, the artificial nucleic acid intron construct is selected from SEQ ID NOS:4-6, 10, 11, 17-20, 24, 28, 32, and 150-157 or has an intron comprising a sequence with at least 70% sequence identity (e.g., about 70%, 75%, 80%, 85%, 90%, 95% or 98% sequence identity) of a sequence selected from SEQ ID NOS:4-6, 10, 11, 17-20, 24, 28, 32, and 150-157. In some embodiments, the sequence identity of the disclosed intron to the reference SEQ ID NOS:4-6, 10, 11, 17-20, 24, 28, 32, and 150-157 is higher at the 5′ end and/or the 3′ end. For example, in some embodiments, the disclosed intron has a 5′ end subsequence with at least 70% sequence identity (e.g., about 70%, 75%, 80%, 85%, 90%, 95% or 98% sequence identity) to the 5′-most 15 nucleotide positions of one of SEQ ID NOS:4-6, 10, 11, 17-20, 24, 28, 32, and 150-157. In some embodiments, the disclosed intron has a 3′ end subsequence with at least 70% sequence identity (e.g., about 70%, 75%, 80%, 85%, 90%, 95% or 98% sequence identity) to the 3′-most 50 nucleotide positions of one of SEQ ID NOS:4-6, 10, 11, 17-20, 24, 28, 32, and 150-157.

In an exemplary group of embodiments, the intron is derived from a human wildtype intron 1 of MTERFD3 (e.g., is derived from an intron sequence comprising a sequence set forth in SEQ ID NO:2). Various additional features of this group of MTERFD3-derived embodiments, which can be included alone or in any combination, are described below.

In a further embodiment, the MTERFD3-derived intron comprises a 5′ splice site comprising a GT dinucleotide immediately followed by a consensus 5′ splice site context. Exemplary 5′ splice site contexts include, but are not limited to AAG, GAG, and GTG, which would result in a sequence of GTAAG, GTGAG, GTGTG, respectively, when including the GT dinucleotide.

In a further embodiment, the canonical 3′ splice site of the MTERFD3-derived intron comprises an AG dinucleotide immediately preceded by a C or T, which would result in a sequence of CAG or TAG, respectively.

In a further embodiment, the MTERFD3-derived intron comprises at least one cryptic 3′ splice site located at least 5 nucleotides upstream of the canonical 3′ splice site. The at least one cryptic 3′ splice site comprises an AG dinucleotide and has a sequence that is a weaker 3′ splice site than is the canonical 3′ splice site. The relative strength or weakness can be estimated computationally, for example with the MaxEntScan algorithm or similar methods.

In a further embodiment, the MTERFD3-derived intron comprises a pyrimidine-rich domain comprising at least 15 consecutive nucleotides. The sequence of the pyrimidine-rich domain is generally at least 50% pyrimidine nucleotides (e.g., including at least about 55%, 60% 65%, 70%, 75%, 80%, 85%, 90%, and 95% pyrimidine nucleotides, as described above). Furthermore, the sequence of the pyrimidine-rich domain is also specifically at least 40% thymine nucleotides (e.g., including at least about 55%, 60% 65%, 70%, 75%, 80%, and 85% thymine nucleotides), which contributes to the pyrimidine content parameter. Additionally, the pyrimidine-rich domain is within at least 30 nucleotides of a cryptic 3′ splice site. As indicated above, the indicated placement refers to the number of intervening nucleotides between and including the closest nucleotide of the pyrimidine-rich domain and the splice site. Thus, a portion of the pyrimidine-rich domain (including a substantial portion) can be outside the indicated range.

In a further embodiment, the MTERFD3-derived intron comprises at least one branchpoint at least 20 nucleotides upstream of the canonical 3′ splice site, such as, e.g., about 25, 30, 35, 40, 45, 50, 55, 60, 65, 70, 75 or more nucleotides upstream of the canonical 3′ splice site.

The embodiments of the intron, including those described above, are configured to be spliced differently in a cell (e.g., cancer cell) comprising a change-of-function or loss-of-function mutation in a recurrently mutated RNA splicing factor gene. The difference in splicing is relative to the splicing pattern of the intron in a cell lacking a change-of-function or loss-of-function mutation in a recurrently mutated RNA splicing factor gene. In some embodiments, the artificial intron is more likely to be recognized and spliced in a cell comprising a change-of-function or loss-of-function mutation in a recurrently mutated RNA splicing factor gene compared to in a cell without the mutation. In some embodiments, the artificial intron is less likely to be recognized and spliced in a cell comprising a change-of-function or loss-of-function mutation in a recurrently mutated RNA splicing factor gene compared to in a cell without the mutation. In some embodiments, the artificial intron is preferentially partially spliced out in a cell comprising a change-of-function or loss-of-function mutation in a recurrently mutated RNA splicing factor gene, such that a portion of the intron is not excised from the mature transcript, while the entire intron is preferentially spliced out in a cell without the mutation. In some embodiments, the entire intron is preferentially spliced out in a cell comprising a change-of-function or loss-of-function mutation in a recurrently mutated RNA splicing factor gene, while the intron is partially spliced out in a cell without the mutation, such that a portion of the intron is not excised from the mature transcript.

There are a variety of RNA splicing factor genes that have recurrent mutations. As used herein, the term “recurrent” refers to a mutation that has been observed in multiple cell types (e.g., multiple cancer types) and/or in multiple individuals with the same cancer type, such that there is an established association with the recurrent mutation and the aberrant phenotype of the cell (e.g., cancer phenotype).

To illustrate, an exemplary and non-limiting RNA splicing factor gene encompassed by this disclosure is SF3B1, which can have a recurrent mutation that leads to a change-of-function or loss-of-function in the expressed splicing factor. Various recurrent mutations in SF3B1 have been previously characterized and are encompassed by this disclosure. For example, in some embodiments, the recurrent change-of-function mutation in SF3B1 leads to one or more of the following changes in the SF3B1 protein sequence (in any combination): E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, and M971V, with respect to the exemplary reference SF3B1 protein sequence set forth in SEQ ID NO:190.

The artificial nucleic acid intron construct can consist of the intron sequence, consist essentially of the intron sequence, or comprise the intron sequence with additional domains or element. For example, in some embodiments, the artificial nucleic acid intron construct comprises the artificial intron, such as described above, in addition to coding sequence flanking one or both ends. In one embodiment, the artificial nucleic acid intron construct further comprises a first exon domain and a second exon domain, wherein the intron is disposed between the first exon domain and the second exon domain. Upon proper splicing, the combination of the first exon domain and the second exon domain in a contiguous sequence, i.e., without the intron) encodes part or all of a protein of interest. In some embodiments, the artificial nucleic acid intron construct can be, comprise, or be comprised in an expression cassette to facilitate transcription. An expression cassette in the present context is a construct that generally includes a gene (e.g., including coding and noncoding, or intron, sequence) and regulatory non-coding sequence to facilitate expression. In some embodiments, the expression cassette comprises a promoter sequence and the gene sequence. In additional embodiments, the expression cassette can further comprise a 5′ untranslated region and/or a 3′ untranslated region.

In some embodiments, the first exon domain is SEQ ID NO:33 and the second exon domain is SEQ ID NO:34, or functional variants thereof. In some embodiments, the first exon domain is SEQ ID NO:35 and the second exon domain is SEQ ID NO:36, or functional variants thereof. In some embodiments, the first exon domain is SEQ ID NO:37 and the second exon domain is SEQ ID NO:38, or functional variants thereof. In some embodiments, the first exon domain is SEQ ID NO:39 and the second exon domain is SEQ ID NO:40, or functional variants thereof. In some embodiments, the first exon domain is SEQ ID NO:41 and the second exon domain is SEQ ID NO:42, or functional variants thereof. In some embodiments, the first exon domain is SEQ ID NO:43 and the second exon domain is SEQ ID NO:44, or functional variants thereof. In some embodiments, the first exon domain is SEQ ID NO:45 and the second exon domain is SEQ ID NO:46, or functional variants thereof. In some embodiments, the first exon domain is SEQ ID NO:47 and the second exon domain is SEQ ID NO:48, or functional variants thereof.

The term “promoter” refers to a regulatory nucleotide sequence that can activate transcription (expression) of a gene. As indicated, a promoter is typically located upstream of a gene, but can be located at other regions proximal to the gene, or even within the gene. The promoter typically contains binding sites for RNA polymerase and one or more transcription factors, which participate in the assembly of the transcriptional complex. As used herein, the term “operatively linked” indicates that the promoter and the gene region (e.g., including coding and noncoding, or intron, sequence) are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended use, as various promoters are known and commonly used in the art. In some embodiments, the nucleic acid intron construct comprises an expression cassette comprising the first exon domain, the intron, the second exon domain, and a promoter sequence operatively linked thereto.

The expression cassette can be incorporated into a vector, such as a plasmid or viral vector, configured for delivery into a cell. Accordingly, in some embodiments, the disclosure provides a vector comprising the artificial nucleic acid intron construct described above. The vector can be any construct that facilitates the delivery of the nucleic acid to the target cell and/or expression of the nucleic acid within the cell. The vectors can be viral vectors, circular nucleic acid constructs (e.g., plasmids), or nanoparticles. Various viral vectors are known in the art and are encompassed by the present disclosure. See, e.g., Machida, C. A. (ed.), Viral Vectors for Gene Therapy: Methods and Protocols, Humana Press, Totowa, New Jersey (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno-associated virus (AAV) vector, an adenovirus vector, a herpes simplex virus vector, a retrovirus vector, a lentivirus vector, an alphavirus vector, a flavivirus vector, a rhabdovirus vector, a measles virus vector, a Newcastle disease virus vector, a Coxsackievirus vector, or a poxvirus vector. An exemplary embodiment of an AAV vector includes the AAV2/5 serotype.

Methods

In another aspect, the disclosure provides a method of generating an artificial nucleic acid intron construct with an intron that can be differentially spliced in a cell depending on the cell's RNA splicing phenotype. The method of this aspect generally comprises:
(1) ligating a 5′ end domain of a human wildtype intron to a 3′ end domain of the human wildtype intron to provide an abbreviated intron that lacks an interior wildtype sequence;
(2) implementing one or more sequence modifications to the abbreviated intron sequence to provide a first plurality of artificial introns derived from the abbreviated intron sequence; and
(3) selecting artificial introns from the first plurality of artificial introns that conform to at least three parameters. Specifically, the artificial introns selected in step (3) are selected if they conform to three, four, or more of the following parameters:
(i) the artificial intron contains a 5′ splice site;
(ii) the artificial intron contains a canonical 3′ splice site;
(iii) the artificial intron contains at least one cryptic 3′ splice site, optionally when the at least one cryptic 3′ splice site is within about 100 nucleotides upstream of the canonical 3′ splice site or within about 50 nucleotides downstream of the canonical 3′ splice site;
(iv) the artificial intron contains a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and
(v) the artificial intron contains at least one branchpoint at least 15 nucleotides upstream of the canonical 3′ splice site.

In some embodiments, the 5′ end domain comprises about 10 to about 150 nucleotides (e.g., about 10, 11, 12, 13, 14, 15, 16, 17, 18, 19, 20, 25, 30, 35, 40, 45, 50, 55, 60, 65, 70, 80, 85, 90, 95, 100, 105, 110, 115, 120, 125, 130, 135, 140, 145, or 150 nucleotides) of the 5′ end sequence of the human wildtype intron, and wherein the 3′ end domain comprises about 50 to about 350 nucleotides (e.g., about 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, or 350 nucleotides) of the 3′ end sequence of the human wildtype intron.

The structural descriptions of the 5′ splice site, the canonical 3′ splice site, the at least one cryptic 3′ splice site, the pyrimidine-rich domain, and the at least one branchpoint provided above in the context of the artificial nucleic acid intron construct apply to the elements of this disclosed method aspect and are not repeated here for brevity.

In some embodiments, the artificial introns selected in step (3) are selected if they conform to parameters (i) and (ii) and further conform to at least two of (iii), (iv), and (v).

Examples of such human source introns from which the disclosed intron can be derived include intron 1 of MTERFD3, intron 4 of MYO15B, intron 10 of SYTL1, intron 11 of SYTL1, intron 4 of MAP3K7, intron 1 of ORAI2, and intron 1 of TMEM14C, although the disclosed intron can be derived from other wildtype introns as well without limitation. Further descriptions of the exemplary, non-limiting introns are provided above in the context of the artificial nucleic acid intron construct. Such descriptions apply here and are not repeated for brevity.

The one or more sequence modifications imposed in step (2) can be any form of sequence modification, such as insertions, deletions, or substitutions, alone or in any combination. Such modifications can be implemented with any technique available in the art without limitation.

Exemplary embodiments of the one or more sequence modifications are now described. The one or more modifications can comprise one or more of the following in any combination and implemented in any order:
(a) mutating a single nucleotide;
(b) mutating any pair of nucleotides within 10 nucleotides of the 5′ end of the abbreviated intron sequence or 30 nucleotides of the 3′ end of the abbreviated intron sequence;
(c) deleting any consecutive stretch of about 5, 10, 15, 20, 25, 30, 35, 40, 45, 50, 100, 125, 150, 200, 250, or more nucleotides, or any number or range contained therein; (d) mutating any pair of nucleotides within the 5 nucleotides upstream of and 2 nucleotides downstream of each branchpoint;
(e) mutating any combination of branchpoints to guanine;
(f) mutating any combination of multiple adenines to guanines;
(g) mutating any one or more branchpoint and flanking sequence contexts to one or more strong branchpoint and flanking sequence contexts;
(h) mutating any four consecutive nucleotides to cAGg;
(i) inserting a polypyrimidine tract immediately followed by a 3′ splice site at any position;
(j) mutating any consecutive stretch of nucleotides to one or more thymines;
(k) mutating all pyrimidines within any six or more consecutive positions to guanines;
(l) inserting a strong branchpoint and flanking sequence context at any position;
(m) inserting one or more intronic splicing enhancers at any position; and
(n) inserting one or more intronic splicing silencers at any position.

In some embodiments, any 2, 3, 4, 5, 6, 7, 8, 9, 10, 11, 12, 13, or all of the modifications (a) through (n) are implemented. Terms such as “upstream”, “downstream”, “branchpoint”, and “pyrimidine” are described in more detail above in the context of the artificial nucleic acid intron construct. Such descriptions apply here and are not repeated for brevity.

In some embodiments, the polypyrimidine tract immediately followed by a 3′ splice site, as described in modification (i), comprises at least six consecutive nucleotides containing at least four pyrimidines. The stretch of the at least four pyrimidines are immediately followed by a sequence selected from AAG, CAG, GAG, TAG, ATG, CTG, GTG, TTG, or any other known 3′ splice site.

The strong branchpoint and flanking sequence context(s) referred to in modifications (g) and (1) can comprise a sequence with a sequence identity of at least 50% (e.g., about 60%, 70%, 80%, 90%) to the sequence tactaAca, where A is a representative branchpoint nucleotide and tacta_ca is a context sequence with a 5′ flanking sequence (tacta) and 3′ flanking sequence (ca). In some embodiments, the A is preserved in the newly imposed strong branchpoint and flanking sequence context.

Intronic splicing enhancers referred to in modification (m) are sequences that facilitate intron recognition and functional splicing within the cell. Any known intronic splicing enhancer sequence can be incorporated into the disclosure without limitation according to ordinary skill and knowledge in the art. For example, see Wang, Y, et al., “Intronic splicing enhancers, cognate splicing factors and context-dependent regulation rules”, Nature Structural & Molecular Biology, 19:1044-1052 (2012), incorporated herein by reference in its entirety. In some embodiments, the one or more intronic splicing enhancers can be selected from GGGTTT, GGTGGT, TTTGGG, GAGGGG, GGTATT, GTAACG, and the like. Intronic splicing silencers referred to in modification (n) are sequences that inhibit intron recognition and functional splicing within the cell. Any known intronic splicing silencer sequence can be incorporated into the disclosure without limitation according to ordinary skill and knowledge in the art. For example, see Wang, Y, et al., Nature Structural & Molecular Biology, 20:36-45 (2013), incorporated herein by reference in its entirety. In some embodiments, the one or more intronic splicing silencers are selected from CACACCA, CTCCTC, TACAGCT, CTTCAG, GAACAG, CAAAGGA, AGATATT, ACATGA, AATTTA, AGTAGG, and the like. The modifications can include a combination of intronic splicing silencers and enhancers according to the needs of the particular application. For example, a particularly strong splice site in a cell with modified RNA splicing functionality (e.g., with mutated RNA splicing factors) might also be susceptible to some residual splicing in cells with normal (e.g., wildtype) RNA splicing factors. Accordingly, an intronic splicing silencer can reduce the likelihood of splicing in the normal cells while permitting an acceptable splicing activity in the cells with modified RNA splicing functionality. A person of ordinary skill in the art can incorporate a combination to balance the enhancing and silencing signals to reach a desired level of differential splicing in the target cells of interest.

An artificial nucleic intron that conforms to the designated parameters can be further incorporated into larger constructs, such as a construct that contains flanking exon sequences containing a protein-coding sequence. In some embodiments, the method further comprises incorporating the artificial nucleic intron into an expression cassette, as described above in more detail. In yet further embodiments, the expression cassette can be incorporated into an expression vector or cell-delivery system to facilitate delivery and expression of the cassette in a target cell. Additional details regarding the expression vectors and cell delivery systems are provided below.

In a related aspect, the disclosure provides an artificial nucleic acid intron construct produced by the method described hereinabove.

In another aspect, the disclosure provides a method of modifying a nucleic acid sequence to permit selective modification of expression in a cell characterized by a mutation in an RNA splicing factor gene. The selective modification of expression can refer to selective expression in the cell, e.g., increased expression in the cell, compared to a cell without the mutation. Increased expression can include any expression in the cell if the reference cell without the expression has no detectable expression. For example, the cell can be a cancer cell with a recurrently mutated RNA splicing factor and the nucleic acid is modified to be selectively expressed to produce a protein in the cancer cell, but to avoid having the production of the protein in non-cancer cells. Alternatively, the selective modification of expression can refer to selective reduction or lack of expression in the cell, compared to a cell without the mutation. For example, the cell can be a cancer cell with a recurrently mutated RNA splicing factor and the nucleic acid is modified to be selectively expressed to produce a protein in the non-cancer cells, but to avoid having the production of the protein in the cancer cells. Furthermore, in the present context, the term “expressed” and grammatical variants thereof refer to successful transcription, processing (including splicing) to produce a mature transcript (i.e., mRNA), and translation of the mature transcript to produce a functional polypeptide molecule (i.e., protein). The artificial nucleic acid introns disclosed herein can modify the expression, i.e., the ultimate production of protein, by being selectively subject to different patterns of splicing (i.e., being selectively susceptible or resistant to excision of the full intron versus excision of none or only part of the intron) from the initial transcribed RNA (i.e., pre-mRNA) before translation occurs.

The method of this aspect comprises the following steps.
(1) Providing a sequence of a target nucleic acid molecule and sequence of an artificial nucleic acid intron as described herein. In some embodiments, the artificial nucleic acid intron is derived from a wildtype intron with known nucleotide sequences of upstream and downstream flanking exons.
(2) Identifying one or more dinucleotides in the target nucleic acid sequence that are identical to an intron dinucleotide sequence. The dinucleotide consists of (from 5′ to 3′) the 3′-most nucleotide of the upstream exon flanking the wildtype intron, and the 5′-most nucleotide of the downstream exon flanking the wildtype intron.
(3) Selecting a dinucleotide identified in step (2) as an insertion point. The insertion point divides the target nucleic acid into a first domain and a second domain. In some embodiments, the first domain and the second domain are substantially the same or similar length. For example, one of the first domain and second domain is at least about 50% (e.g., about 50%, 55%, 60%, 65%, 70%, 75%, 80%, 85%, 90%, 95%) of the length of the other of the first domain and second domain.
(4) Inserting an artificial intron molecule with the artificial nucleic acid intron sequence between the first domain and the second domain of the target nucleic acid molecule.

In some embodiments, the selecting activity in step (3) further comprises the following design steps:
(a) computationally inserting the sequence of the artificial nucleic acid intron at the selected insertion point to create a hypothetical exonic flanking sequence context for a 5′ splice site and a 3′-most 3′ splice site;
(b) computing strength scores for the 5′ splice site and the 3′-most 3′ splice site, respectively, in their hypothetical exonic contexts;
(c) comparing the computed strength scores for the 5′ splice site and 3′-most 3′ splice site within their hypothetical exonic contexts to strength scores of the respective 5′ splice site and 3′-most 3′ splice site of the wildtype intron in its wildtype exonic context from which the artificial nucleic acid intron is derived; and
(d) selecting a dinucleotide wherein computational insertion of the artificial nucleic acid intron sequence results in strength scores for the 5′ splice site and 3′-most 3′ splice site in their hypothetical exonic contexts that differ by about 50% or less of the respective 5′ splice site and 3′-most 3′ splice site scores of the wildtype intron in its wildtype exonic context (i.e., the scores in the hypothetical exonic contexts are between 50% and 150% of the respective scores in the wildtype exonic context).

Strength scores can be computed using any available program or algorithm that models splicing performance. For example, in some non-limiting embodiments, the strength scores can be computed with a standard method such as MaxEntScan::scores5ss, MaxEntScan::score3ss, HumanSplicingFinder, and other similar algorithms known in the art. See, e.g., Desmet, et al., Human Splicing Finder: an online bioinformatics tool to predict splicing signals, Nucleic Acids Res. 2009 May; 37(9): e67; and Yeo, G. and Burge C., Maximum entropy modeling of short sequence motifs with applications to RNA splicing signals, J Comput Biol. 2004; 11(2-3):377-94, each of which is incorporated herein by reference in its entirety.

In some embodiments, the selecting activity in step (3) can further comprise introducing one or more synonymous codon mutations into the nucleic acid that improve or weaken one or both scores for the 5′ and 3′-most 3′ splice sites in their hypothetical exonic contexts. Synonymous codon mutations are substitutions in the encoding DNA sequence that encode for the same amino acid (i.e., are redundant to) the original sequence. By this approach, the relative splice strength scores can be adjusted as necessary for the desired application of the artificial intron construct.

In some embodiments, the method can further comprise introducing one or more synonymous codon mutations into the nucleic acid that result in creation of one or more exonic splicing enhancers and/or one or more exonic splicing silencers. As indicated above, exonic enhancers and/or silencers can be incorporated to fine tune the construct's susceptibility to splicing. A practitioner might include an exonic splicing enhancer if the artificial intron construct is not effectively spliced out at high rates even in target cells, e.g., cells with recurrent mutation in an RNA splicing factor gene. Conversely, a practitioner might incorporate an exonic splicing silencer if the artificial intron is spliced out at high rates in the target cells, including at unacceptable rates in wildtype cells without the RNA splicing factor gene mutation (e.g., wildtype reference cells). Sequences serving as splicing enhancers or splicing silencers are described in more detail above and are encompassed by this aspect of the disclosure. In some embodiments, the one or more exonic splicing enhancers is/are selected from CCNG, CGNG, GCNG, GGNG, and other sequences with known enhanced likelihood of binding by serine/arginine-rich (SR) proteins, in any combination. The designation of N refers to any nucleotide. In some embodiments, the one or more exonic splicing silencers is/are selected from TTTGTTCCGT (SEQ ID NO:160), GGGTGGTTTA (SEQ ID NO:161), GTAGGTAGGT (SEQ ID NO:162), TTCGTTCTGC (SEQ ID NO:163), GGTAAGTAGG (SEQ ID NO:164), GGTTAGTTTA (SEQ ID NO:165), TTCGTAGGTA (SEQ ID NO:166), GGTCCACTAG (SEQ ID NO:167), TTCTGTTCCT (SEQ ID NO:168), TCGTTCCTTA (SEQ ID NO:169), GGGATGGGGT (SEQ ID NO:170), GTTTGGGGGT (SEQ ID NO:171), TATAGGGGGG (SEQ ID NO:172), GGGGTTGGGA (SEQ ID NO:173), TTTCCTGATG (SEQ ID NO:174), TGTTTAGTTA (SEQ ID NO:175), TTCTTAGTTA (SEQ ID NO:176), GTAGGTTTG, GTTAGGTATA (SEQ ID NO:177), TAATAGTTTA (SEQ ID NO:178), TTCGTTTGGG (SEQ ID NO:179), and the like, or sequences with at least 50% identity thereto. See, e.g., Wang, Z., et al., Systematic Identification and Analysis of Exonic Splicing Silencers, Cell, 119(6):831-845, 2004, incorporated herein by reference in its entirety, for disclosure of exonic splicing silencers encompassed by the present disclosure.

In some embodiments, the disclosed steps are performed multiple times for a given target nucleic acid molecule such that two or more (e.g., 3, 4, 5, 6, or more) artificial intron molecules are ultimately inserted into the target nucleic acid molecule. The insertion of the two or more artificial introns results in a plurality of target molecule domains, wherein each of the plurality of target molecule domains are separated by the artificial intron molecules. The plurality of target molecule domains can each correspond to a different portion of the same CDS. The plurality of separated target molecule domains can be of any size in relation to each other. In some embodiments, however, each of the plurality of the separated target molecule domains is at least about 50% (e.g., about 50%, 55%, 60%, 65%, 70%, 75%, 80%, 85%, 90%, 95%) of the length of the longest separated target molecule domain.

The target nucleic acid molecule can an isolated nucleic acid molecule with a protein-coding sequence (CDS) that encodes a protein of interest. The target nucleic acid modified with the artificial intron construct molecule is configured to permit selective modified expression (e.g., selective increased expression, or alternately selective lack of expression) of the protein of interest in a cell characterized by a mutation in an RNA splicing factor gene. As indicated above, the term selective refers to the modified expression (e.g., increased or lack of expression) in the cell characterized by a mutation in an RNA splicing factor gene in contrast to reference cells characterized by the wildtype RNA splicing factor gene. As described above, the term expression refers to the ultimate production of a protein product translated from a gene transcript. The expression involves proper splicing of the intron construct to permit expression of the final protein product. The artificial intron construct can be configured for selective proper splicing by the cell in the context of the mutated RNA splicing factor, or alternatively to selectively prevent proper splicing by the cell in the context of the mutated RNA splicing factor.

In some embodiments, the method further comprises introducing the modified target nucleic acid molecule to a cancer cell with a mutation in an RNA splicing factor gene and permitting expression, or alternately selective lack of expression, of the protein of interest. The modified target nucleic acid molecule can be incorporated into a functional expression cassette, as described above. In some embodiments, the modified target nucleic acid molecule is incorporated into an expression vector, such as a viral expression vector, or other cell delivery/expression system, as described herein, to promote delivery into and expression in the cancer cell.

In some embodiments, the target nucleic acid molecule is a gene in the chromosome of a cell, wherein the gene encodes a protein of interest, and the modified target nucleic acid molecule is configured for selective expression, or alternately selective lack of expression, in a cell characterized by a mutation in an RNA splicing factor gene.

In some embodiments, the cell is a cancer cell and the mutation in an RNA splicing factor gene is a change-of-function or loss-of-function mutation in a recurrently mutated RNA splicing factor gene. As indicated above, the artificial intron sequence is configured to be spliced differently in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene. The different splicing pattern of the artificial intron sequence results in production of different mature transcripts of the modified target nucleic acid molecule in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene. The production of different mature transcripts of the modified nucleic acid molecule permits either selective expression, or alternately selective lack of expression, of a desired protein from the target nucleic acid molecule in the cancer cell, and the opposite pattern in a cell lacking the change-of-function or loss-of-function mutation in the recurrently mutated RNA splicing factor gene.

RNA splicing factor genes that are subject to recurrent mutations are known. As used herein, the term “recurrent mutation” and grammatical variants thereof refer to the mutation (or mutations) being observed in multiple individuals such that there is an association between the mutation and the altered functionality of the RNA splicing factor expressed from the mutated gene. In some embodiments, the mutation (or mutations) are associated with or demonstrably contribute to the phenotype of a transformed (e.g., cancer) cell. One illustrative, non-limiting example of the RNA splicing factor gene is SF3B1, which is known to have recurrent mutations associated with change of function. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190.

In another aspect, the disclosure provides a method of selectively expressing, or alternately selectively not expressing, a gene of interest in a cell. The cell comprises a change-of-function or loss-of-function mutation in a recurrently mutated RNA splicing factor gene.

The method comprises introducing to the cell an expression cassette comprising a coding sequence (CDS) interrupted by at least one artificial nucleic acid intron described hereinabove. The expression cassette further comprises a promoter operatively linked to the CDS. The terms “promoter” and “operatively linked” are defined above. The method further comprises permitting transcription of the coding sequence and modified splicing of the transcript induced by the artificial nucleic acid intron in the resulting transcript in conjunction with the mutated splicing factor. As described above, the modified splicing of the transcript can encompass an increased likelihood of a splicing event such that the resulting protein is expressed, or an decreased likelihood of a splicing event such that the resulting translation product is not the protein in its functional form. The modification is selective in that the outcome is specific to the cell(s) with the cell comprising a change-of-function or loss-of-function mutation in a recurrently mutated RNA splicing factor gene in comparison to a cell without the change-of-function or loss-of-function mutation. It will be appreciated that while the RNA splicing factor expressed from the mutated RNA splicing factor gene is necessary for the modified splicing of the artificial intron, it does not itself perform the direct catalytic reaction of splicing. Instead, the mutated splicing factor alters splice site, intron, or exon recognition to allow subsequent splicing of the artificial intron domain by other factors. Alternately, for the case of recurrent loss-of-function mutations in an RNA splicing factor gene, absence of the functional RNA splicing factor results in modified recognition or loss of recognition of splice sites, introns, or exons.

The expression cassette can be incorporated into an expression vector, such as a viral expression vector, or other cell delivery/expression system, as described herein, to promote delivery into and expression in the cell.

The cell can be a cancer cell and the mutation in an RNA splicing factor gene can be a change-of-function or loss-of-function mutation in a recurrently mutated RNA splicing factor gene, as described above. As described above, an exemplary and non-limiting RNA splicing factor gene encompassed by the disclosure is SF3B1. Exemplary recurrent change-of-function mutations in SF3B1 protein sequence include E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, which are encompassed in the present disclosure, individually or in any combination, with respect to the amino acid sequence set forth in SEQ ID NO:190.

The cancer cell can be from any cancer, myelodysplastic syndrome or other hematologic disease, or other dysplastic, proliferative, or malignant disease that is characterized by or associated with a recurrently mutated RNA splicing factor gene. For example, with respect to recurrent mutations in SF3B1, the cancer is a myelodysplastic syndrome (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, or other neoplasm with recurrent SF3B1 mutations.

In some embodiments, upon splicing of the at least one artificial nucleic acid intron from the gene transcript, the mature transcript, i.e., with the CDS lacking the intron, encodes a functional therapeutic protein. The functional therapeutic protein can be any protein that, when expressed, can have a detrimental effect on the cancer cell, whether directly or indirectly, alone or in conjunction with other therapeutics or immune system factors. For example, the functional therapeutic protein can be a toxin, chemokine, cytokine, growth factor, targetable cell-surface protein, targetable antigen, druggable enzyme, detectable marker, and the like. Exemplary functional therapeutic proteins are described in more detail below.

In another aspect, the disclosure provides a method of treatment in a subject for a subject with cancer. The method incorporates cancer-specific gene therapy. In this aspect, the cancer is characterized by a change-of-function or loss-of-function mutation in a recurrently mutated RNA splicing factor gene, as have been described above. The method comprises administering to the subject an effective amount of a therapeutic composition comprising an expression cassette comprising a coding sequence (CDS) interrupted by at least one artificial nucleic acid intron as described herein. The expression cassette further comprises a promoter operatively linked to the CDS, as described herein.

As described above, an exemplary and non-limiting RNA splicing factor gene encompassed by the disclosure that can have recurrent mutations is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, and combinations thereof, with reference to the wild-type amino acid sequence set forth in SEQ ID NO:190.

The cell can be from any cancer, myelodysplastic syndrome or other hematologic disease, or other dysplastic, proliferative, or malignant disease that is characterized by or associated with a recurrently mutated RNA splicing factor gene. For example, with respect to recurrent mutations in SF3B1, the cancer is a myelodysplastic syndrome (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, or other neoplasm with recurrent SF3B1 mutations.

In some embodiments, upon splicing of the at least one artificial nucleic acid intron from the gene transcript, the mature transcript, i.e., with the CDS lacking the intron, encodes a functional therapeutic protein. The functional therapeutic protein can be any protein that, when expressed, can have a detrimental effect on the cancer cell, whether directly or indirectly, alone or in conjunction with other therapeutics or immune system factors. For example, the functional therapeutic protein can be a toxin, chemokine, cytokine, growth factor, targetable cell-surface protein, targetable antigen, druggable enzyme, detectable marker, and the like. Exemplary functional therapeutic proteins are now described.

In some embodiments, the functional therapeutic protein is a chemokine, cytokine, or growth factor, and wherein the chemokine, cytokine, or growth factor stimulates an increased immune response against the cancer cell. As non-limiting examples, the functional therapeutic protein can be IFN alpha, IFN beta, IFN gamma, IL-2, IL-12, IL-15, IL-18, IL-24, TNF-alpha, GM-CSF, and the like, or functional domains or derivatives thereof. Exemplary cytokines and derivatives are known (see, e.g., Levin, A. M., et al. Exploiting a natural conformational switch to engineer an interleukin-2‘superkine’. Nature, 484(7395), 529-533 (2012) and Silva, D. A., et al. De novo design of potent and selective mimics of IL-2 and IL-15. Nature, 565(7738), 186-191 (2019), incorporated herein by reference in its entirety) and are encompassed by the disclosure. In one embodiment, the functional therapeutic protein is IL-2 or IL-2-derived variant proteins, such as IL-2 “superkines,” that exhibit desirable therapeutic properties such as enhanced activation of cytotoxic CD8⁺ T cells. For example, as described in Example 2 below, a CDS for IL-2 was divided by the disclosed artificial introns in an expression cassette. When transcribed and properly spliced in leukemic and melanoma cells with a change of function mutation in the RNA splicing factor gene SF3B1, the exons are combined in the mRNA leading to proper expression and secretion of the IL-2 protein by the cells. Exemplary IL-2 exon sequences that can be used in conjunction with the disclosed artificial introns to implement such cell-specific expression of functional IL-2 protein are set forth as SEQ ID NOS: 148 and 149 (for use with, e.g., synMTERFD3i1 family introns). Use of these disclosed exons, or exons with sequences that encode the same protein sequences encoded by SEQ ID NOS:148 and 149, in an expression cassette with the disclosed artificial intron constructs, and functional variants and derivatives thereof, are encompassed by the disclosure.

In some embodiments, the functional therapeutic protein is a targetable cell-surface protein or targetable antigen. In further embodiments, the method further comprises administering to the subject an effective amount of a second therapeutic composition comprising an affinity reagent that specifically binds the target cell-surface protein or targetable antigen. Useful targetable antigens include proteins that are not typically expressed in healthy cells, or not typically expressed at high levels in healthy cells, such that a targeting affinity reagent will bind with substantial specificity to the transformed cell induced to express the targetable antigen. Non-limiting examples of targetable cell-surface proteins or targetable antigens include CD19, CD22, CD23, CD123, ROR1, truncated EGFR (EGFRt), or functional domains thereof, and the like.

As used herein, the term “affinity reagent” refers to a molecule that specifically binds to a target antigen, and typically a specific epitope on a target antigen. As used herein, the term “specifically bind” or variations thereof refer to the ability of the affinity reagent(s) to bind to the antigen of interest (e.g., the targetable antigen or cell-surface protein), without significant binding to other molecules, under standard conditions known in the art.

Exemplary, non-limiting categories of affinity reagent include antibodies, an antibody-like molecule (including antigen-binding fragments of antibodies and derivatives thereof), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional antigen-binding domain or fragment thereof.

In some embodiments, the indicated affinity reagent is an antibody. As used herein, the term “antibody” encompasses antibodies and antigen-binding antibody fragments or derivatives thereof, derived from any antibody-producing mammal (e.g., mouse, rat, rabbit, camel, and primate including human), that specifically bind to an antigen of interest (e.g., the targetable antigen or cell-surface protein). Exemplary antibodies include multi-specific antibodies (e.g., bispecific antibodies); humanized antibodies; murine antibodies; chimeric, mouse-human, mouse-primate, primate-human monoclonal antibodies; and anti-idiotype antibodies. The antigen-binding molecule can be any intact antibody molecule or fragment or derivative thereof (e.g., with a functional antigen-binding domain).

An antibody fragment is a portion derived from or related to a full-length antibody, preferably including the complementarity-determining regions (CDRs), antigen-binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagents do not have to be naturally occurring or naturally derived, but can be further modified to, e.g., reduce the size of the domain or modify affinity for the antigen (e.g., the targetable antigen or cell-surface protein) as necessary. For example, complementarity-determining regions (CDRs) can be derived from one source organism and combined with other components of another, such as human, to produce a chimeric molecule.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Bispecific antibodies can incorporate CDR regions of two different identified monoclonal antibodies by fusing encoding gene portions for the relevant binding domains followed by cloning into an expression vector that also comprises nucleic acids encoding the remaining structure(s) of the bispecific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

The affinity reagent employed as the agent can also be an aptamer. As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phosphodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer-antigen binding.

Peptide aptamers are protein molecules that often contain a peptide loop attached at both ends to a protein scaffold. The loop typically is between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

The affinity reagents can be configured to carry a toxic payload that is detrimental to the cell with induced expression of the targetable antigen or cell surface protein. Alternatively, the affinity reagent can be configured to induce an immune response against the cell with induced expression of the targetable antigen or cell-surface protein.

In some embodiments, the second therapeutic composition comprises an antibody, or a fragment or derivative thereof. In other embodiments, the second therapeutic composition comprises an immune cell expressing an antibody, or fragment or derivative thereof, or an immune cell expressing a T cell receptor, or fragment or derivative thereof. The expressed antibody or T cell receptor, or fragment or derivative thereof, specifically binds the antigen. For example, in some embodiments, the immune cell expresses a chimeric antigen receptor with an antigen-binding domain and an intracellular domain that induces a response by the immune cell upon binding of the antigen-binding domain to the antigen or cell-surface receptor whose expression is selectively induced in the cancer cell.

In some embodiments, the functional therapeutic protein is a toxin. Any toxin that is locally detrimental or lethal to the expressing cell is encompassed by this disclosure. Some non-limiting examples include Caspase 9, TRAIL, Fas ligand, and the like, or functional fragments thereof.

In some embodiments, the functional therapeutic protein is a druggable enzyme. A druggable enzyme is an enzyme that is ideally not substantially prevalent in healthy cells, but when expressed presents a target for a known therapeutic, which can be additionally administered to the specific detriment of the cancer cell expressing the druggable enzyme target. Various druggable enzymes and their associated therapeutics are known and are encompassed by this disclosure. Non-limiting examples are provided below.

In one embodiment, the druggable enzyme is herpes simplex virus thymidine kinase and the method further comprises administering to the subject an effective amount of ganciclovir. For example, as described in Example 1 below, a CDS for herpes simplex virus thymidine kinase (HSV-TK) was divided by the disclosed artificial introns in an expression cassette. When transcribed and properly spliced in leukemic and melanoma cells with a change of function mutation in the RNA splicing factor gene SF3B1, the exons are combined in the mRNA leading to proper expression of the HSV-TK protein in the cells. Upon treatment with ganciclovir, the cells are selectively killed compared to cells not properly expressing the HSV-TK (i.e., cell not receiving the expression cassette or cells with receiving the expression cassette but having wild-type SF3B1). Exemplary HSV-TK exon sequences that can be used in conjunction with the disclosed artificial introns to implement such cell-specific expression of functional HSV-TK protein are set forth as SEQ ID NOS:35 and 36 (for use with, e.g., synMTERFD3i1 family introns), and 43 and 44 (for use with, e.g., synMAP3K7i4 family introns). Use of these disclosed exons, or exons that encode the same amino acid sequences as SEQ ID NOS:35 and 36 (or SEQ ID NOS:43 and 44), in an expression cassette with the disclosed artificial intron constructs, and functional variants and derivatives thereof, are encompassed by the disclosure.

In one embodiment, the druggable enzyme is cytosine deaminase and the method further comprises administering to the subject an effective amount of 5-fluorocytosine. In one embodiment, the druggable enzyme is nitroreductase and the method further comprises administering to the subject an effective amount of CB1954 or analogs thereof. In one embodiment, the druggable enzyme is carboxypeptidase G2 and the method further comprises administering to the subject an effective amount of CMDA, ZD-2767P, and the like. In one embodiment, the druggable enzyme is purine nucleoside phosphorylase and the method further comprises administering to the subject an effective amount of 6-methylpurine deoxyriboside, and the like. In one embodiment, the druggable enzyme is cytochrome P450 and the method further comprises administering to the subject an effective amount of cyclophosphamide, ifosfamide, and the like. In one embodiment, the druggable enzyme is horseradish peroxidase and the method further comprises administering to the subject an effective amount of indole-3-acetic acid, and the like. In one embodiment, the druggable enzyme is carboxylesterase and the method further comprises administering to the subject an effective amount of irinotecan, and the like.

In some embodiments, the functional therapeutic protein is a detectable marker and can be useful in monitoring and/or guiding surgical procedures in the removal of the cancer cells. In some embodiments, the detectable marker provides a visual detectable signal (e.g., fluorescent signal) and the method further comprises surgically removing the cancer cells expressing the detectable marker. For example, as described in Example 1 below, a CDS for mEmerald was divided by the disclosed artificial introns in an expression cassette. When transcribed and properly spliced in target cancer cells with a change of function mutation in the RNA splicing factor gene SF3B1, the exons are combined in the mRNA leading to proper expression of the mEmerald protein in the cells. As a result, the cells are selectively fluorescent compared to cells not properly expressing the mEmerald protein (i.e., cell not receiving the expression cassette or cells with receiving the expression cassette but having wild-type SF3B1). Exemplary mEmerald exon sequences that can be used in conjunction with the disclosed artificial introns to implement such cell-specific expression of functional mEmerald protein are set forth as SEQ ID NOS:33 and 34 (for use with, e.g., synMTERFD3i1 family introns); SEQ ID NOS:37 and 38 (for use with, e.g., synMYO15Bi4 family introns); SEQ ID NOS:39 and 40 (for use with, e.g., synSYTL1i10 family introns); SEQ ID NOS:41 and 42 (for use with, e.g., synMAP3K7i4 family introns); SEQ ID NOS:45 and 46 (for use with, e.g., synORAI2i1 family introns); and SEQ ID NOS:47 and 48 (for use with, e.g., synTMEM14Ci1 family introns). Use of these disclosed exons in an expression cassette with the disclosed artificial intron constructs, and functional variants and derivatives thereof, are encompassed by the disclosure.

In yet other embodiments, multiple therapeutic proteins are simultaneously expressed. For example, as described in Example 2 below, a CDS for herpes simplex virus thymidine kinase (HSV-TK) was divided by the disclosed artificial introns in an expression cassette. This divided HSV-TK CDS was then immediately followed by a 2A peptide and a CDS for IL-2. When transcribed and properly spliced in leukemic and melanoma cells with a change of function mutation in the RNA splicing factor gene SF3B1, the HSV-TK exons are combined in the mRNA leading to proper expression of the HSV-TK protein as well as expression and secretion of the IL-2 protein in the cells. Exemplary HSV-TK exon sequences that can be used in conjunction with the disclosed artificial introns to implement such cell-specific expression of functional HSV-TK protein are set forth as SEQ ID NOS:35 and 36 (for use with, e.g., synMTERFD3i1 family introns), and 43 and 44 (for use with, e.g., synMAP3K7i4 family introns). An exemplary 2A CDS that can be used to implement such cell-specific expression is set forth as SEQ ID NOS:147, although other 2A CDSs (e.g., from foot-and-mouth disease virus, equine rhinitis A virus, Thosea asigna virus, and porcine teschovirus-1) are known and can be used. An exemplary IL-2 CDS is set forth as SEQ ID NO:146. Use of these disclosed sequences, individually or in combination, in an expression cassette with the disclosed artificial intron constructs, and functional variants and derivatives thereof, are encompassed by the disclosure.

The therapeutic compositions and/or additional therapeutic agents described herein can be formulated for any local or systemic mode of administration to facilitate efficient delivery and, with respect to the disclosed therapeutic composition with the artificial intron construct, expression in the target cells.

In some embodiments, the artificial nucleic acid intron construct, and expression cassette comprising a coding sequence (CDS) interrupted by at least one artificial nucleic acid intron, is comprised in a vector, e.g., viral expression vector, that facilitates expression of the heterologous nucleic acid in the nucleus of the target cell. In some embodiments, the vector promotes integration of the heterologous nucleic acid in the genome of the cell.

As indicated above, the construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses (AAV)), adenovirus, coronavirus, Newcastle disease virus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (see, e.g., Coffin, J. M., Retroviridae: The viruses and their replication, In Fundamental Virology, Third Edition, B. N. Fields et al., Eds., Lippincott-Raven Publishers, Philadelphia, 1996, incorporated herein by reference in its entirety).

As used herein, “expression vector” refers to a DNA construct containing a nucleic acid molecule that is operatively-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host cell, the vector may replicate and function independently of the host genome, or may, in some instances, integrate into the genome itself. In the present specification, “plasmid,” “expression plasmid,” “virus” and “vector” can be used interchangeably.

In some embodiments, the therapeutic composition further comprises a vehicle for intracellular delivery and a pharmaceutically acceptable carrier. The vehicle can be a liposome, nanocapsule, nanoparticle, exosome, microparticle, microsphere, lipid particle, vesicle, and the like, configured for the introduction of the expression cassette into cancer cells.

In some embodiments, the therapeutic composition further comprises a non-viral gene editing system and a pharmaceutically acceptable carrier. Chromosomal editing can be performed using, for example, endonucleases. As used herein, “endonuclease” refers to an enzyme capable of catalyzing cleavage of a phosphodiester bond within a polynucleotide chain. In certain embodiments, an endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule, such as the artificial synthetic introns herein, may be used for a donor gene “knock-in”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a FokI endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double-strand break (DSB) in the genome, and targeted integration of a transgene comprising flanking sequences homologous to the genome at the site of DSB is facilitated by homology-directed repair. Alternatively, a DSB generated by a ZFN can result in knock out of a target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entireties). TALENs can be used to direct site-specific double-strand breaks (DSBs) in the genomes of cells. Non-homologous end joining (NHEJ) ligates DNA from both sides of a double-strand break in which there is little or no sequence overlap for annealing, thereby introducing errors that knock out gene expression. Alternatively, homology-directed repair can introduce a transgene at the site of DSB, providing homologous flanking sequences are present in the transgene. In certain embodiments, a gene knockout comprises an insertion, a deletion, a mutation or a combination thereof, made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. The type II system, the most studied, comprises at least three components: an RNA-guided Cas9 nuclease, a crRNA, and a trans-acting crRNA (tracrRNA). The tracrRNA comprises a duplex-forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology-directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programmed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference in its entirety). In certain embodiments, a gene knockout comprises an insertion, a deletion, a mutation or a combination thereof, made using a CRISPR/Cas nuclease system.

As used herein, a “meganuclease,” also referred to as a “homing endonuclease,” refers to an endodeoxyribonuclease characterized by a large recognition site (double-stranded DNA sequences of about 12 to about 40 base pairs). Meganucleases can be divided into five families based on sequence and structure motifs: LAGLIDADG (SEQ ID NO:50), GIY-YIG (SEQ ID NO:51), HNH, His-Cys box and PD-(D/E)XK (SEQ ID NO:52). Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J Mol. Biol. 263:163-180, 1996; Argast et al., J. Mol. Biol. 280:345-353, 1998, each of which is incorporated herein by reference in its entirety).

As indicated above, the CDS generated by splicing the artificial intron can be a protein that provides a detectable signal. The selective expression of such a reporter protein in a cancer cell can be leveraged to guide more specific and targeted surgical techniques. Accordingly, in another aspect, the disclosure provides a method of enhancing surgical resection of a tumor from a subject. In this aspect, the tumor is characterized by a change-of-function or loss-of-function mutation in a recurrently mutated RNA splicing factor gene. The method comprises administering to the subject an effective amount of a therapeutic composition comprising an expression cassette comprising a coding sequence (CDS) encoding a detectable marker, wherein the CDS is interrupted by at least one artificial nucleic acid intron as described above, and wherein the expression cassette further comprises a promoter operatively linked to the CDS.

In one embodiment, the RNA splicing factor gene is SF3B1. Exemplary recurrent change-of-function mutations in the SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190, and are encompassed in this aspect, individually or in any combination. Cancer types associated with recurrent change-of-function mutations in RNA splicing factor genes, such as SF3B1, are known and are encompassed by this aspect of the disclosure. Exemplary cancer types include uveal melanoma, mucosal melanoma, skin melanoma, breast cancer, pancreatic cancer, endometrial cancer, liver cancer, lung cancer, mesothelioma, or other solid tumor or neoplasm with recurrent SF3B1 mutations.

In some embodiments, the detectable marker is a fluorescent or luminescent protein. For example, any fluorescent protein at any detectable spectrum (blue/uv, cyan, green, yellow, orange, red, far-red, etc.) can be used. See, e.g., Snapp E. Design and use of fluorescent fusion proteins in cell biology. Curr Protoc Cell Biol. 2005; Chapter 21:21.4.1-21.4.13. doi:10.1002/0471143030.cb2104s27, incorporated herein by reference in its entirety. Non-limiting examples of fluorescent and luminescent proteins include TagBFP2, BFP, mTurquoise2, TagGFP2, GFP, eGFP, Superfolder GFP, TurboGFP, mEmerald, Azamin Green, mTFP1 (Teal), EYFP, Topaz, T-Sapphire, mWasabi, mVenus, mKO, EBFP, ABFP2, Azurite, mTagBFP, ECFP, Cerulean, mTurquoise, CyPet, AmCyan1, Midori-Ishi Cyan, TagCFP, mCitrine, YPet, TagYFP, PhiYFP, ZsYellow1, mBanana, mOrange, dTomato, TagRFP, DsRed/2, mTangerine, mRuby, mStrawberry, Jred, mRaspberry, mPlum, mApple, mCherry, mKate2, Katushka, mCardinal, firefly luciferase, and renilla luciferase, and the like.

The method can further comprise the step of detecting fluorescent or luminescent tumor cells and surgically resecting the fluorescent or luminescent tumor cells.

The expression cassette can be disposed in a vector, e.g., a viral vector, or otherwise formulated with a vehicle (e.g., nanoparticle, liposome, etc.) for intracellular delivery, as described above in more detail.

In another aspect, the disclosure provides an in vitro method of screening candidate compositions for activity in a cell. The cell has a genetic background comprising a change-of-function or loss-of-function mutation in a recurrently mutated RNA splicing factor gene. The cells can be established transformed cell lines with known genetic backgrounds or can be cells derived from a subject with a suspected genetic background that comprises a change-of-function or loss-of-function mutation in a recurrently mutated RNA splicing factor gene. For example, in some embodiments, the RNA splicing factor gene is SF3B1. Illustrative, non-limiting examples of the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190.

The method comprises contacting the cell with an expression cassette comprising a coding sequence (CDS) interrupted by at least one artificial nucleic acid intron, as disclosed herein. The cell is contacted with a candidate composition and transcription from the expression cassette, with any transcriptional processing (i.e., RNA splicing), is permitted. The cells are monitored for modulation of the expression of a functional reporter protein, which indicates whether the candidate composition modulates the activity of the recurrently mutated RNA splicing factor. In some embodiments, the modulation is the presence or increase of functional reporter protein when a mutated RNA splicing factor is present and functionally active. In alternative embodiments, the modulation is the decrease or absence of functional reporter protein in when a mutated RNA splicing factor is present and functionally active.

The expression cassette can comprise a promoter and/or appropriate enhancers operatively linked to the CDS. Upon processing of the transcript encoded, and potential splicing of the artificial nucleic acid intron, the CDS encodes or does not encode a functional detectable reporter protein. Splicing depends upon mutant splicing factor activity in the cell and, therefore, differs between cells with a genetic background comprising a change-of-function or loss-of-function mutation in a recurrently mutated RNA splicing factor gene and cells lacking such a mutation.

For example, as described in Example 3 below, a CDS for mEmerald was divided by the disclosed artificial intron in an expression cassette. When transcribed and properly spliced in breast epithelial and melanoma cells, which possess a change of function mutation in the RNA splicing factor gene SF3B1, the exons were combined in the mRNA leading to expression of intact mEmerald protein by the cells. In contrast, cells lacking such a mutation in SF3B1 did not express mEmerald, which replicated the effect of a compound that successfully modulates (i.e., inhibits) the activity of the recurrently mutated RNA splicing factor. This difference between cells with or without aberrant SF3B1 splicing activity is readily detectable as a difference in the relative fluorescence signal.

In some embodiments, detection of a functional reporter protein or a relative increase of functional reporter protein in the cell indicates the candidate composition does not suppress activity of the mutated RNA splicing factor in the cell. In contrast, detection of an absence or relative reduction in functional reporter protein in the cell indicates the candidate composition does suppress activity of the mutated RNA splicing factor in the cell.

The screen can be scaled up to assess the impact of a library of candidate compounds on aberrant RNA splicing due to change-of-function or loss-of-function mutation(s) in a recurrently mutated RNA splicing factor gene. The screen can be characterized as a positive screen, i.e., assessing for a positive effect in inhibiting aberrant RNA splicing. In some embodiments, indicated above, the cells are derived from a subject, e.g., from a biopsy. The screen can be implemented to assess how the suspected cancer in the subject might respond to a variety of candidate therapeutics. For example, the cells can be expanded and arranged in an array plate and individual cells or groups of cells are transformed with the expression cassette comprising the artificial intron and contacted with different potential therapeutics. In some embodiments, the detection of reporter protein is indicative of the aberrant splicing activity and, thus, is inversely proportional to the efficacy of the therapeutic contacted to the cells.

In other embodiments, the screen can be characterized as a negative screen. The expression cassette comprising the synthetic intron can be configured, as described above, to preferentially result in expression of a functional reporter protein in the absence of a mutated RNA splicing factor or in the presence of an inhibited mutated RNA splicing factor. Accordingly, detection of a functional reporter protein in the cell indicates the candidate composition suppresses activity of the mutated RNA splicing factor in the cell. In contrast, an absence or relative reduction in detected functional reporter protein in the cell indicates the candidate composition does not suppress activity of the mutated RNA splicing factor in the cell.

The step of detecting the presence of a functional reporter protein can comprise quantifying the amount of reporter protein. This can be performed according to standard techniques in the art, and depends on the nature of the reporter protein incorporated into the method.

Reporter proteins, and their sequences, appropriate for these methods are well-known in the art and are encompassed by the present disclosure. A nonlimiting list of exemplary reporter proteins are described above. In some embodiments, the reporter protein is a fluorescent protein or a luminescent protein. Other reporter proteins can be enzymatic proteins, such as β-galactosidase, that catalyze reactions that can be readily assayed.

In some embodiments, the method further comprises contacting a control cell without a change-of-function or loss-of-function mutation in a recurrently mutated RNA splicing factor gene with the expression cassette and further contacting the control cell with the candidate composition. This can provide a reference or standard reporter protein level to which the experimental screen results can be compared.

The candidate composition can be any composition suspected of having a potential direct or indirect effect on the transcription or splicing functionality in a cell. For example, the candidate composition can be selected from a small molecule, protein (e.g., antibody, or fragment or derivative thereof, enzyme, and the like), and nucleic acid construct to alter the genome or transcriptome of the cell, or a complex of a nucleic acid and protein.

In some embodiments, the nucleic acid construct is an interfering RNA construct. In other embodiments, the candidate composition comprises a Transcription Activator-Like Effector Nuclease (TALEN), Zinc Finger Nuclease (ZFN), or recombinant fusion protein. In some embodiments, the candidate composition comprises a guide nucleic acid specific for a target sequence and an associated nuclease that modifies and/or cleaves a nucleic acid molecule upon binding of the guide nucleic acid to its target sequence. Alternatively, the candidate composition comprises a guide nucleic acid specific for a target sequence and an associated catalytically inactive nuclease, wherein binding of the guide nucleic acid to the target sequence results in modification of transcription, splicing, or translation of the target sequence. In further embodiments, the associated nuclease is Cas9, Cas12, Cas13, Cas14, variants thereof, and the like.

In a similar aspect, the disclosure provides a method of screening a cell with suspected genetic background comprising a change-of-function or loss-of-function mutation in a recurrently mutated RNA splicing factor gene. The cell can be derived from a subject, e.g., a suspected cancer cell obtained from the subject. As above, the cell is contacted with an expression cassette comprising a coding sequence (CDS) interrupted by at least one artificial nucleic acid intron, as disclosed herein. The cell is monitored for expression of an intact protein resulting from a complete CDS, e.g., an intact reporter protein, which indicates aberrant activity of an RNA splicing factor and, thus, indicates the presence of a change-of-function or loss-of-function mutation in a recurrently mutated RNA splicing factor gene, as described above. Cells that exhibit aberrant RNA splicing, as indicated by presence of an protein encoded by the CDS, can be further subjected to a screen of candidate compounds that may inhibit aberrant RNA splicing to determine the appropriateness of the candidate compounds as a therapeutic.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., a cancer), or any combination thereof. The term “therapeutic effect” refers to the reduction, elimination, or prevention of the disease or condition, symptoms of the disease or condition, or side effects of the disease or condition in the subject.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

### Abstract

Many cancers carry recurrent, change-of-function mutations affecting RNA splicing factors, which induce sequence-specific splicing alterations (see, e.g., Yoshida, K. et al. Frequent pathway mutations of splicing machinery in myelodysplasia. Nature 478, 64-69 (2011); Papaemmanuil, E. et al. Somatic SF3B1 mutation in myelodysplasia with ring sideroblasts. The New England journal of medicine 365, 1384-1395 (2011); Quesada, V. et al. Exome sequencing identifies recurrent mutations of the splicing factor SF3B1 gene in chronic lymphocytic leukemia. Nature genetics (2011) doi:10.1038/ng.1032; Graubert, T. A. et al. Recurrent mutations in the U2AF1 splicing factor in myelodysplastic syndromes. Nature genetics (2011) doi:10.1038/ng.1031; Dvinge, H., et al. RNA splicing factors as oncoproteins and tumour suppressors. Nature reviews. Cancer 16, 413-430 (2016); and Wang, L. et al. SF3B1 and other novel cancer genes in chronic lymphocytic leukemia. The New England journal of medicine 365, 2497-2506 (2011), each of which is incorporated herein by reference in its entirety).

This Example describes a method to harness this abnormal splicing activity to drive splicing factor mutation-dependent gene expression in cancers and selectively eliminate these tumors. Synthetic introns were engineered that were efficiently spliced in cancer cells bearing SF3B1 mutations, but unspliced in otherwise isogenic wild-type cells, to yield mutation-dependent protein production. A massively parallel screen of 8,878 introns delineated ideal intronic size and mapped essential sequence elements underlying mutation-dependent splicing. Synthetic introns enabled mutation-dependent expression of herpes simplex virus thymidine kinase and subsequent ganciclovir-mediated elimination of SF3B1-mutant cancer cells, while leaving wild-type cells unaffected. This approach dramatically decreased the growth of otherwise lethal leukemia, breast cancer, and uveal melanoma xenografts and correspondingly improved host survival. The modular, compact, and specific nature of synthetic introns provides a power platform for inducing gene expression in selectively in specific cancer cells. This can be leveraged, for example, as a means to exploit cancer-specific changes in RNA splicing for gene therapy, among other applications.

### Results and Discussion

Recurrent mutations affecting an RNA splicing factor occur in many cancer types, with frequencies ranging from 65-83% in myelodysplastic syndromes with ring sideroblasts (MDS-RS) and 14-29% in uveal melanoma to 15-35% in acute myeloid leukemia (AML) and 2-3% in breast adenocarcinoma. These lesions are attractive targets for therapeutic development thanks to their pan-cancer nature, frequent occurrence as initiating or early events, presence in the dominant clone, and particular enrichment in diseases with few effective therapies. Accordingly, several studies have demonstrated that cancer cells bearing spliceosomal mutations are preferentially sensitive to further splicing perturbation, including treatment with compounds that inhibit normal spliceosome assembly or function. However, the therapeutic index of drugs that inhibit global splicing activity is not yet clear. Moreover, therapeutic approaches that target the function of the mutant splicing machinery itself have not yet been identified.

Spliceosomal mutations alter splice site and exon recognition to cause dramatic mis-splicing of a restricted set of genes, while leaving most genes unaffected. Although these splicing changes promote aberrant self-renewal, transformation, and other pro-tumorigenic phenotypes, the inventors hypothesized that this splicing dysregulation could be exploited for therapeutic development. Accordingly, synthetic constructs were designed, developed and tested for differential splicing in cells with or without recurrent mutations in SF3B1, the most commonly mutated spliceosomal gene in cancer, to allow for cancer cell-specific protein production.

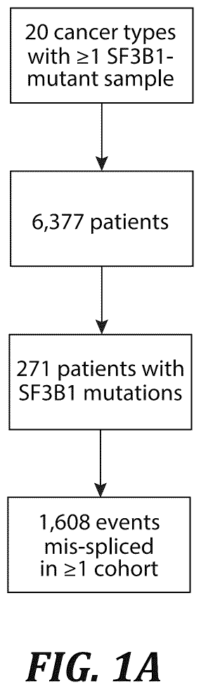

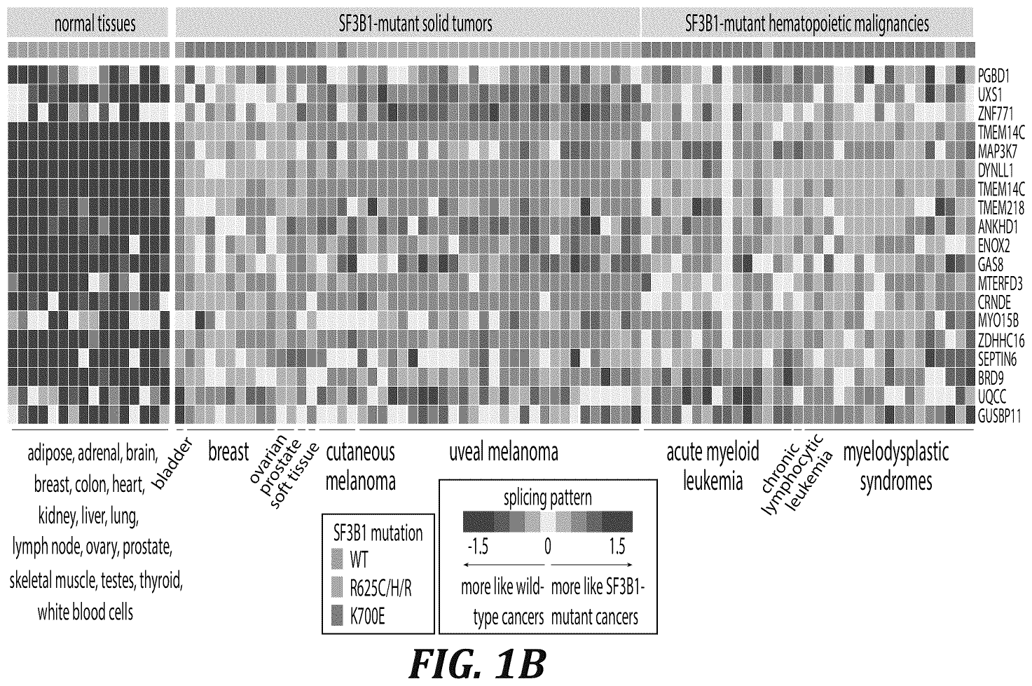

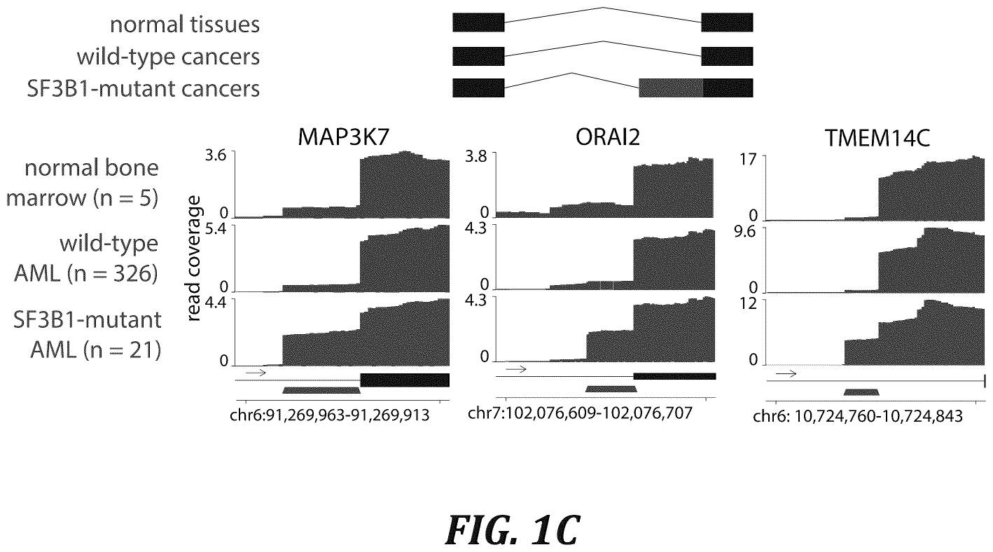

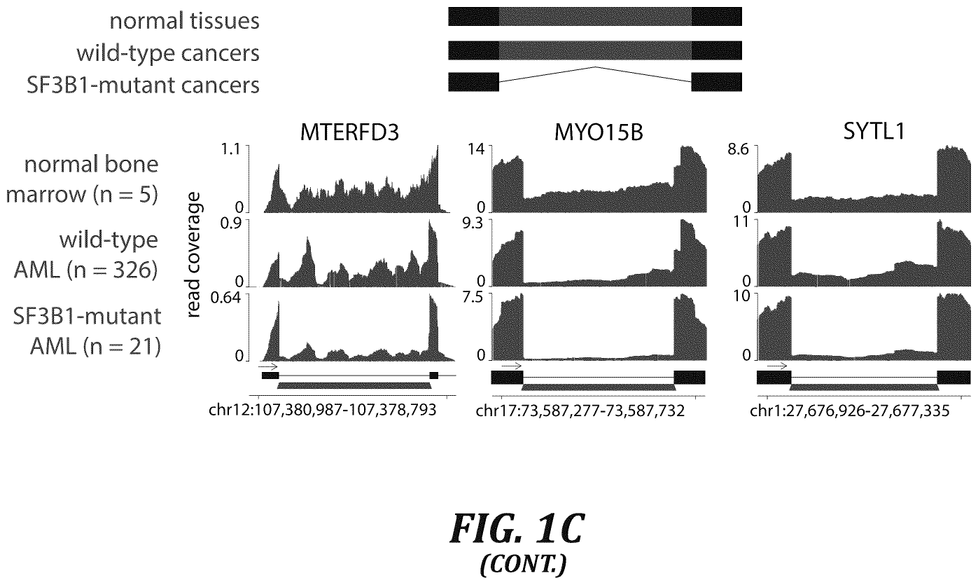

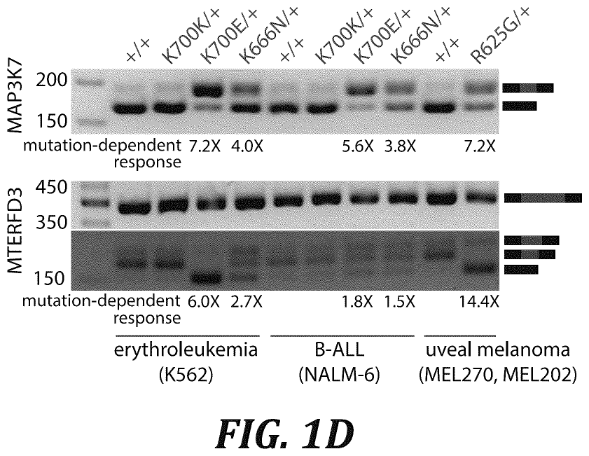

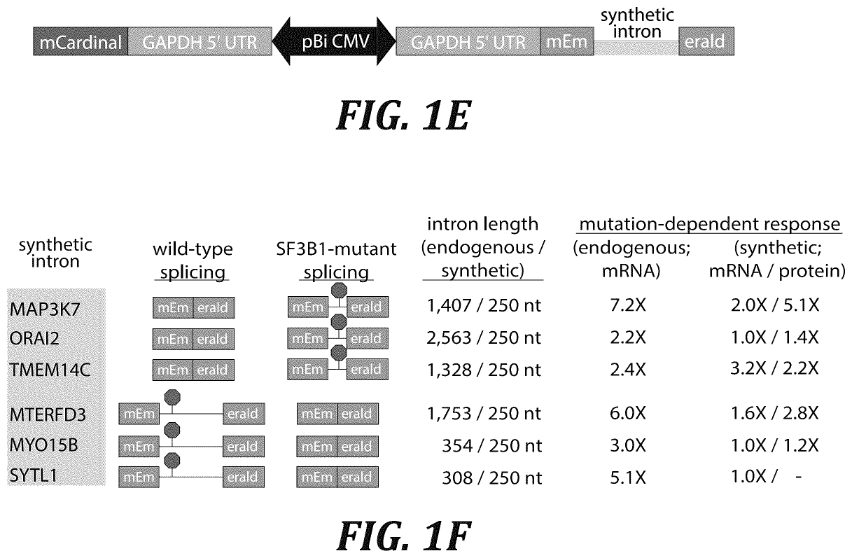

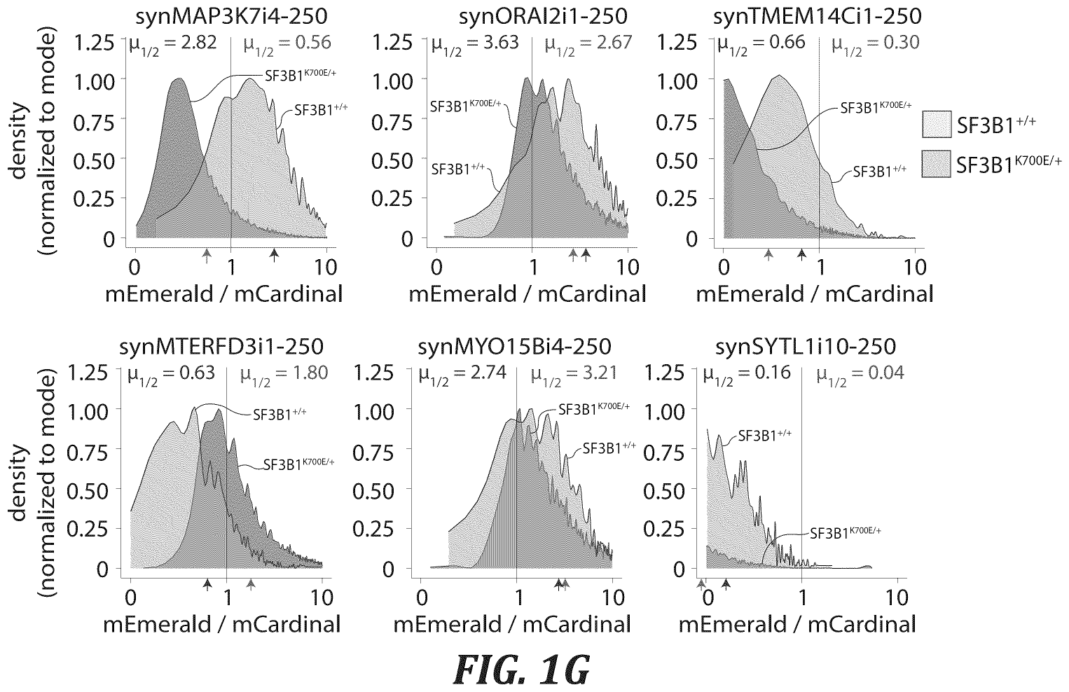

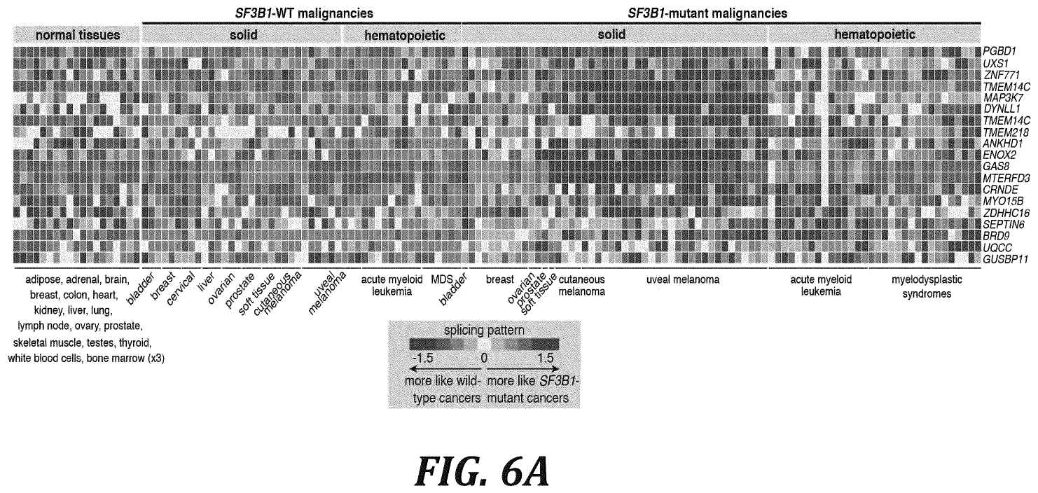

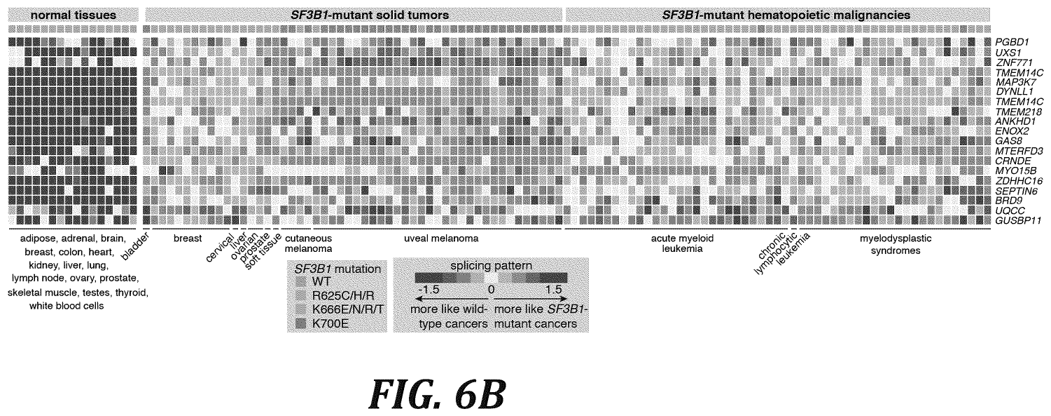

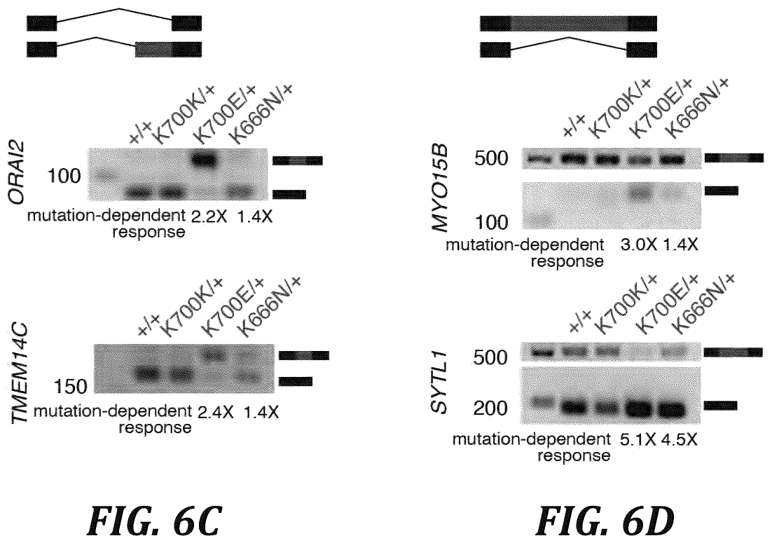

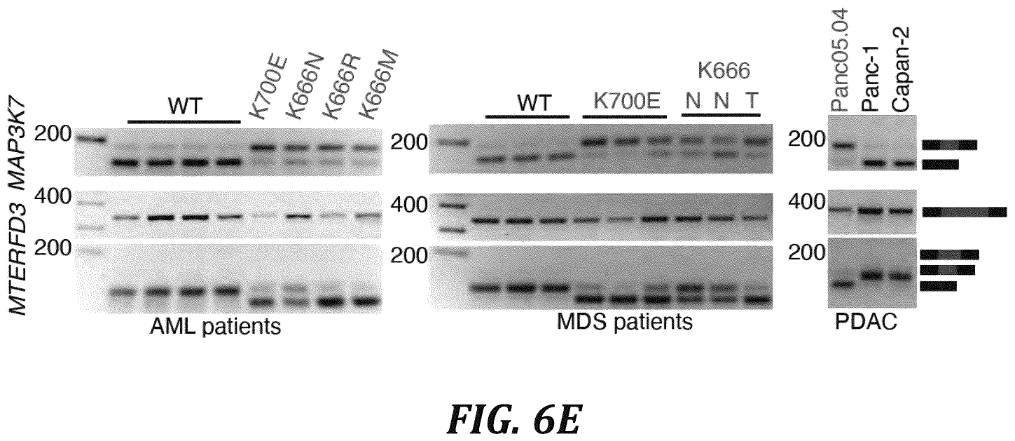

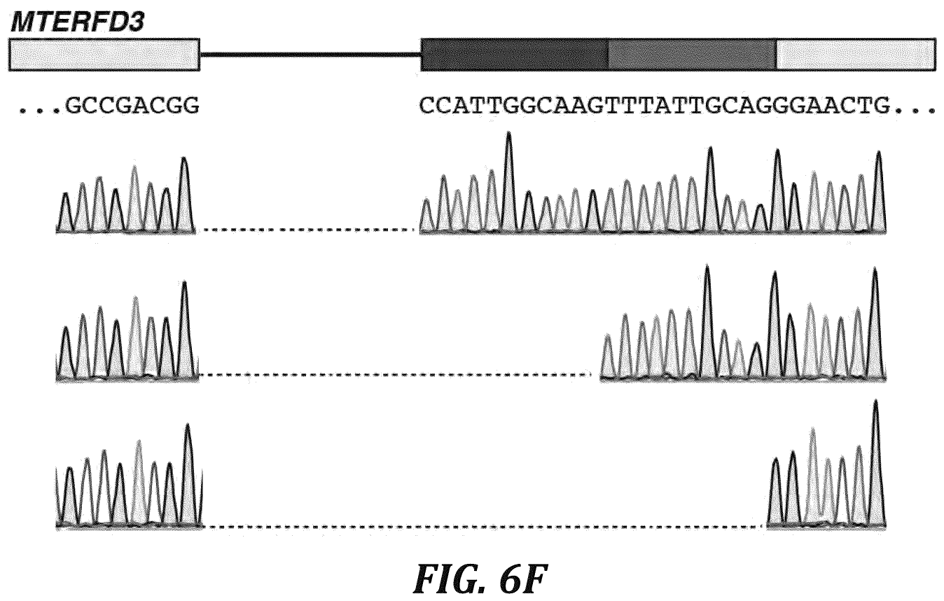

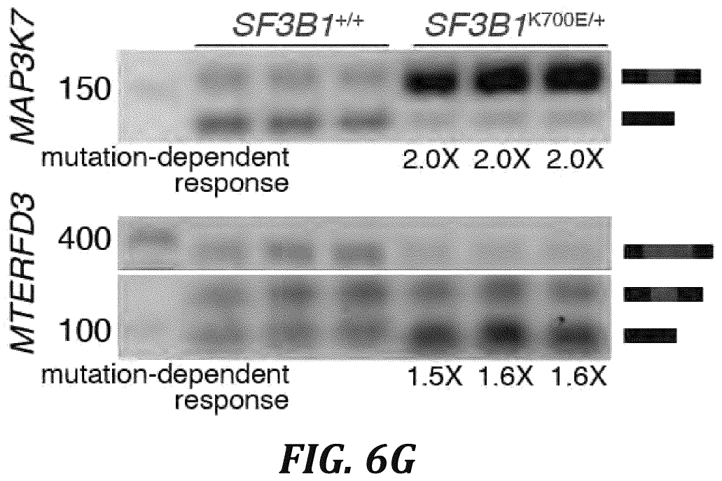

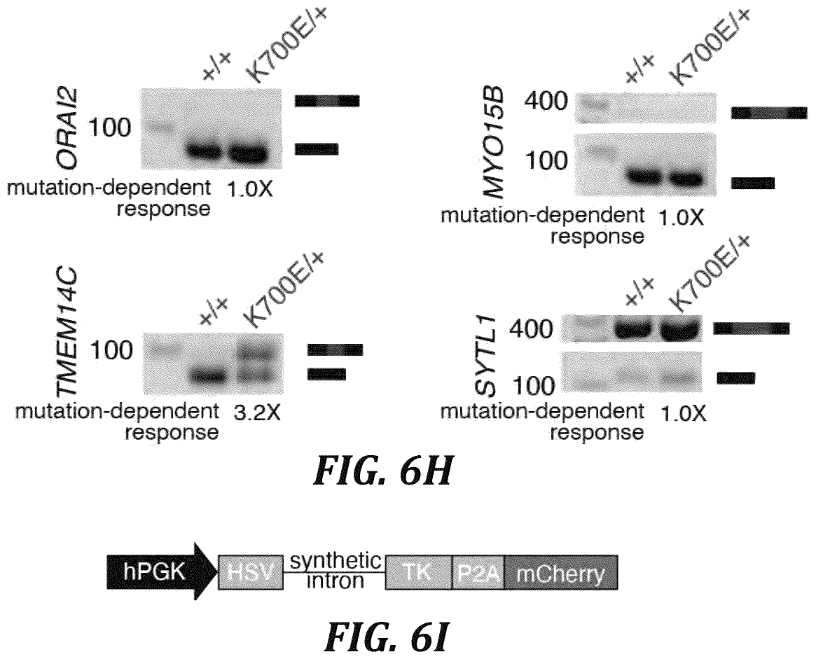

Endogenous genes were first identified that responded most strongly and consistently to SF3B1 mutations, which are near-universally present as heterozygous, missense changes affecting a few residues. The transcriptomes of 35 cancer cohorts were queried to identify 20 distinct cancer types with more than one SF3B1-mutant sample, with a total of 271 samples from patients carrying SF3B1 mutations (sample origins in Data Availability). 1,608 splicing events were significantly differentially spliced between samples bearing no spliceosomal mutations (wild-type; WT) and SF3B1-mutant samples in at least one cohort, with a subset exhibiting highly consistent differential splicing (FIGS. 1A, 1B, 6A, and 6B). SF3B1 mutations were associated with diverse splicing changes, including altered 3′ splice site (3′ss) selection, exon recognition, and intron retention.

Six introns representing two major classes of splicing events were selected for further study. SF3B1 mutations activate intron-proximal cryptic 3′ss in MAP3K7, ORAI2, and TMEM14C, and promote efficient intron removal in MTERFD3, MYO15B, and SYTL1 (FIG. 1C). These were among the strongest and most consistent mis-splicing events, and preferentially caused either open reading frame disruption (MAP3K7, ORAI2, and TMEM14C) or preservation (MTERFD3, MYO15B, and SYTL1) in SF3B1-mutant samples. The mis-splicing observed in primary patient samples was confirmed to be recapitulated in isogenic engineered K562 (erythroleukemic) and NALM-6 (B-cell acute lymphoblastic leukemia) cells with or without SF3B1K666N and SF3B1K700E mutations; in MEL270 and MEL202 (uveal melanoma) cells, which have endogenous WT or mutant (R625G) SF3B1; and in primary samples from patients with AML or MDS with WT or mutant (K666M/N/R/T, K700E) SF3B1 (FIGS. 1D, and 6C-6E). These experiments revealed particularly complex splicing for MTERFD3; three distinct spliced isoforms were observed in addition to the intron retention isoform evident from RNA-seq. Therefore, each isoform were cloned and sequenced to identify three distinct, competing 3′ splice sites within the MTERFD3 intron (FIG. 6F).

These six endogenous introns served as starting points for the development of synthetic introns that functioned as compact and modular molecular switches. Each intron was reduced to 250 nt in length by taking the first 100 and last 150 nt, and inserted into the mEmerald coding sequence in a location that preserved the 5′ss and 3′ss strengths of the endogenous genes as well as generated exons of roughly comparable sizes. These choices were guided by the increased complexity of the 3′ss versus 5′ss, SF3B1's functional role in 3′ss recognition, and the importance of exon length in splicing. Each split mEmerald sequence was cloned into a vector with constitutive expression of the non-overlapping fluorophore mCardinal (FIG. 1E). The resulting vectors permitted quantitative assessment of mutation-dependent protein production by measuring the ratio of mEmerald to mCardinal in cells with or without an SF3B1 mutation via flow cytometry.

Each construct was transfected into isogenic WT or SF3B1-mutant K562 cells and mutation-dependent splicing and protein production was measured. Of the six initial synthetic introns, three exhibited mutation-dependent specificity of 2-fold (synMAP3K7i4-250, synTMEM14Ci1-250, and synMTERFD3i1-250) and two others drove modestly mutation-dependent protein production (synORAI2i1-250 and synMYO15Bi4-250; FIGS. 1F and 1G). Mutation-dependent protein production arose from mutation-dependent splicing changes, as designed (FIGS. 6G and 6H). These proof-of-principle studies confirmed the feasibility of using synthetic introns for mutation-dependent gene expression.

Next, the therapeutic potential of using synthetic introns to achieve mutation-dependent toxin delivery to cancer cells was tested. The herpes simplex virus thymidine kinase (HSV-TK) system was selected, in which treatment of HSV-TK-expressing cells with the prodrug ganciclovir (GCV) causes cytotoxic metabolite production (Smith, K. O., Galloway, K. S., Kennell, W. L., Ogilvie, K. K. & Radatus, B. K. A new nucleoside analog, 9-[[2-hydroxy-1-(hydroxymethyl)ethoxyl]methyl]guanine, highly active in vitro against herpes simplex virus types 1 and 2. Antimicrob Agents Ch 22, 55-61 (1982), incorporated herein by reference in its entirety), was selected. As GCV is an FDA-approved antiviral therapy with low toxicity for cells lacking HSV-TK, HSV-TK is an attractive system for cancer gene therapy.

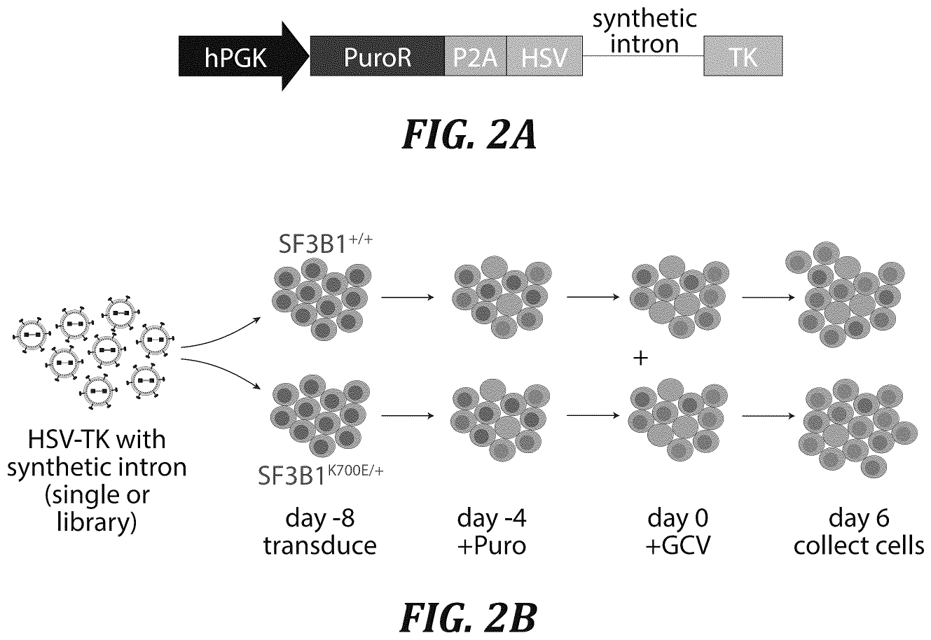

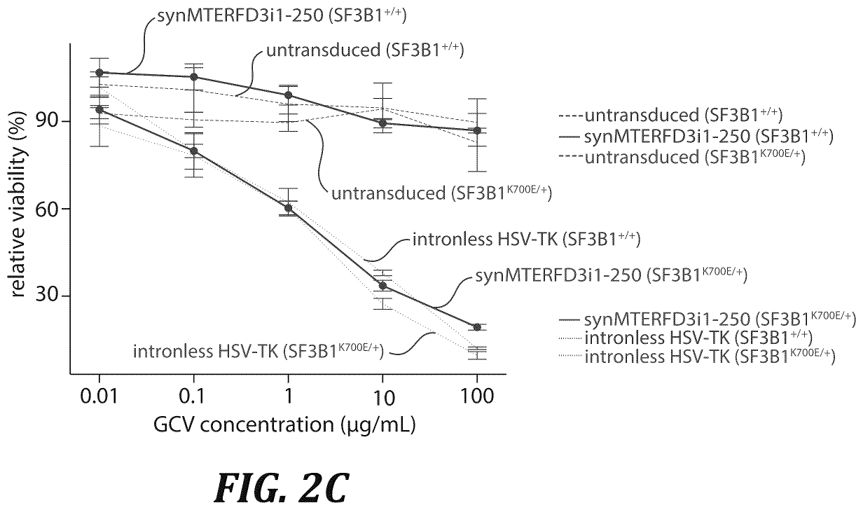

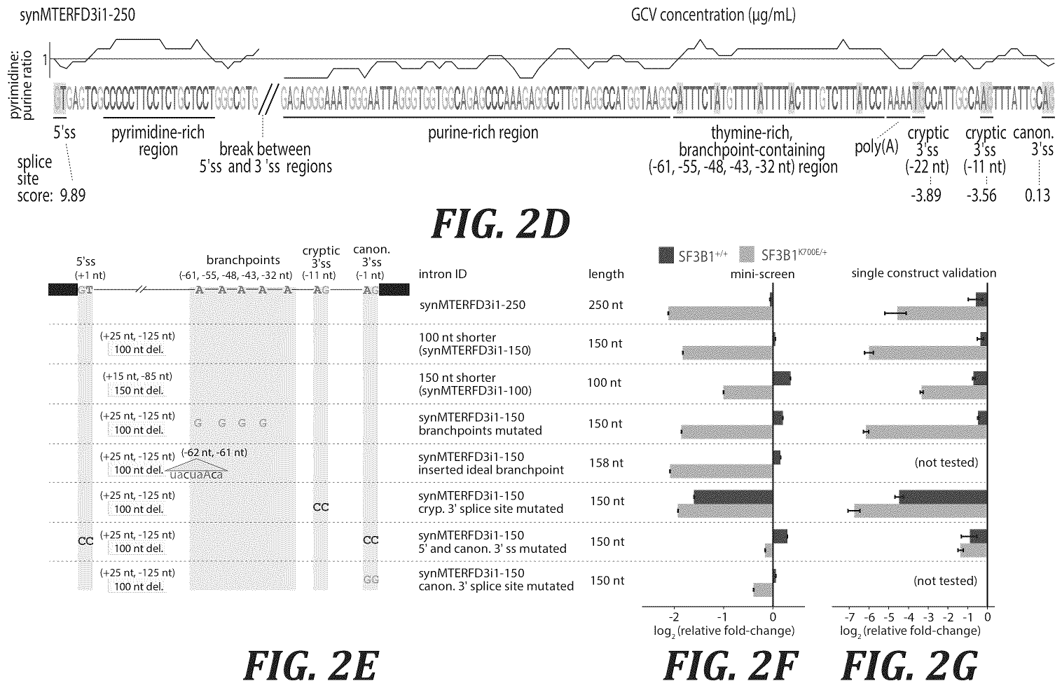

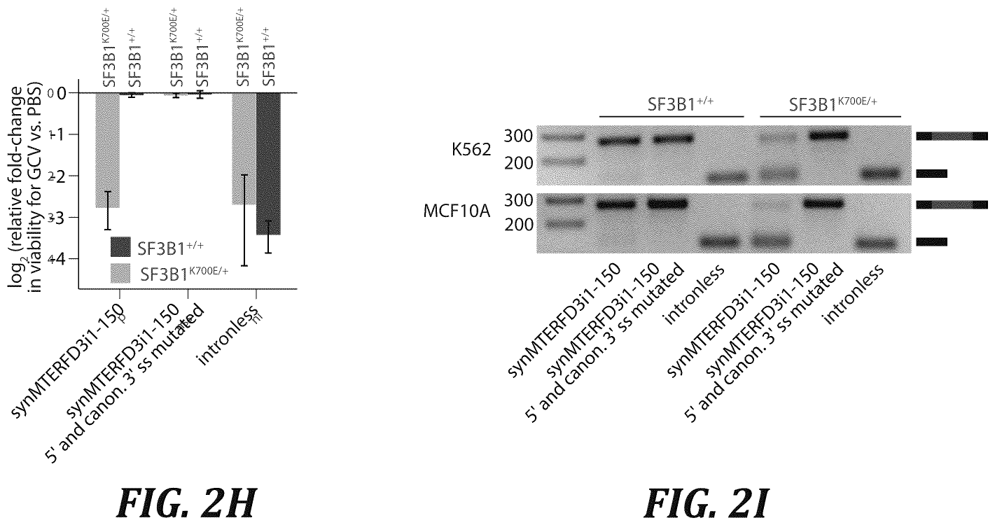

Following the same approach used for fluorescent protein expression, the MTERFD3-derived synthetic intron, which was more efficiently excised in SF3B1-mutant cells, was inserted into the HSV-TK coding sequence (FIGS. 2A and 6I). This split HSV-TK sequence or an intronless HSV-TK was cloned into a lentiviral expression vector. Isogenic WT or SF3B1-mutant K562 cells were infected. Positive integrants were selected and treated with GCV (FIG. 2B). Untransduced cells exhibited minimal loss of viability, while cells transduced with an intronless HSV-TK construct died rapidly, independent of SF3B1 mutational status. SF3B1-mutant cells expressing synthetic intron-containing HSV-TK exhibited a rapid and dose-dependent loss of viability, indistinguishable to that caused by intronless HSV-TK; in contrast, WT cells expressing synthetic intron-containing HSV-TK exhibited no significant differences in viability from untransduced cells (FIG. 2C).

Next key sequence features were identified that conferred mutation responsiveness to the described synthetic intron with sequence analysis, cDNA cloning, and branchpoint identification. The intron has a simple 5′ss region, with a near-consensus 5′ss followed by a pyrimidine-rich region of unknown function. In contrast, its 3′ss region is very complex. It has two cryptic 3′ss at positions −11 and −22 relative to the canonical (frame-preserving) 3′ss, with a highly unusual TG dinucleotide at the most intron-proximal site. The cryptic 3′ss at −22 nt is followed immediately by a short poly(A) sequence of unknown function, which in turn is followed by a thymine-rich region that resembles a polypyrimidine tract interrupted by branchpoints. Five branchpoints were identified at positions −32, −43, −48, −55, and −61, corresponding to all adenines within the thymine-rich region. This thymine-rich, branchpoint-containing region is immediately followed by a long, purine-rich region of unknown function (FIG. 2D). Because of the intron's high complexity, the sequence features that govern mutation responsiveness were not a priori obvious.

Therefore, a massively parallel splicing assay was developed to map and functionally interrogate key sequence features within the synthetic intron. First, a pilot mini-library of eight synthetic introns was synthesized, seven of which were much shorter (lengths of 100-158 nt) than the parent synthetic intron (synMTERFD3i1-250), and each with one or more perturbations to potentially critical features (FIG. 2E). This mini-library was cloned into HSV-TK, introduced into WT or SF3B1-mutant K562 cells with a lentiviral vector at a low multiplicity of infection, and treated with GCV. Relative depletion of each construct was measured by high-throughput sequencing of the entire introns from genomic DNA after six days of treatment (TABLE 1).

This pilot experiment demonstrated the utility of parallel screening for functional interrogation (FIG. 2F). The parent synthetic intron (synMTERFD3i1-250) was markedly depleted in SF3B1-mutant, but not WT, cells. Shortening the intron to 150 or 100 nt resulted in robust or modest mutation responsiveness, respectively. Mutation responsiveness was maintained even after ablating all four commonly used branchpoints or inserting a single consensus branchpoint at the 5′ end of the thymine-rich, branchpoint-containing region upstream of the 3′ss. Removing either the 5′ss or canonical (frame-preserving) 3′ss prevented introns from becoming depleted even in SF3B1-mutant cells, as expected for abolition of splicing, while removing the cryptic 3′ss at position −11 resulted in strong depletion irrespective of genotype.

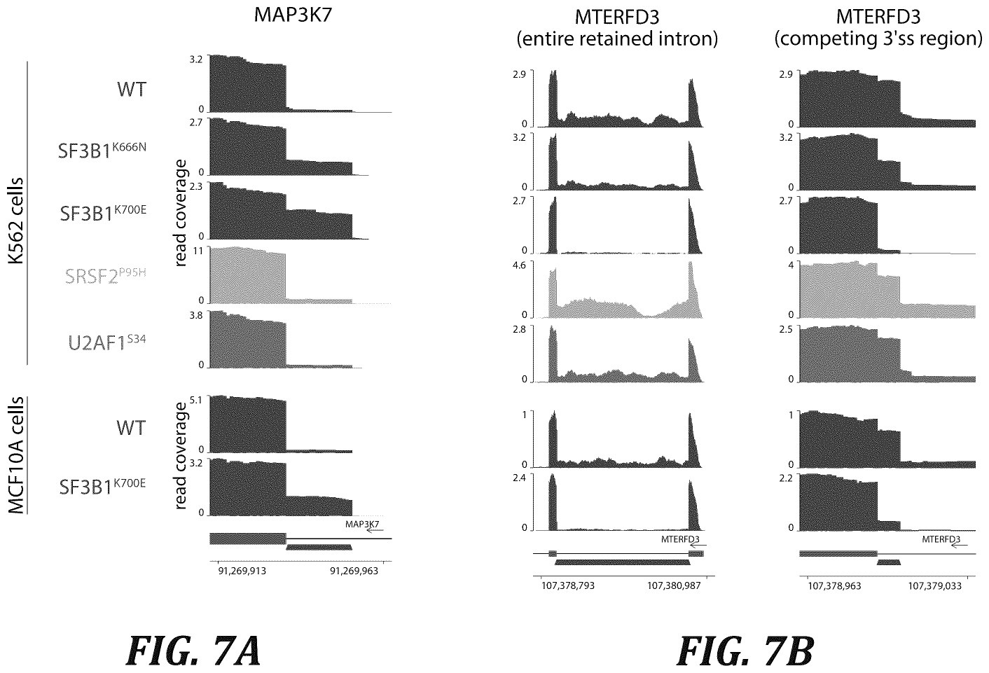

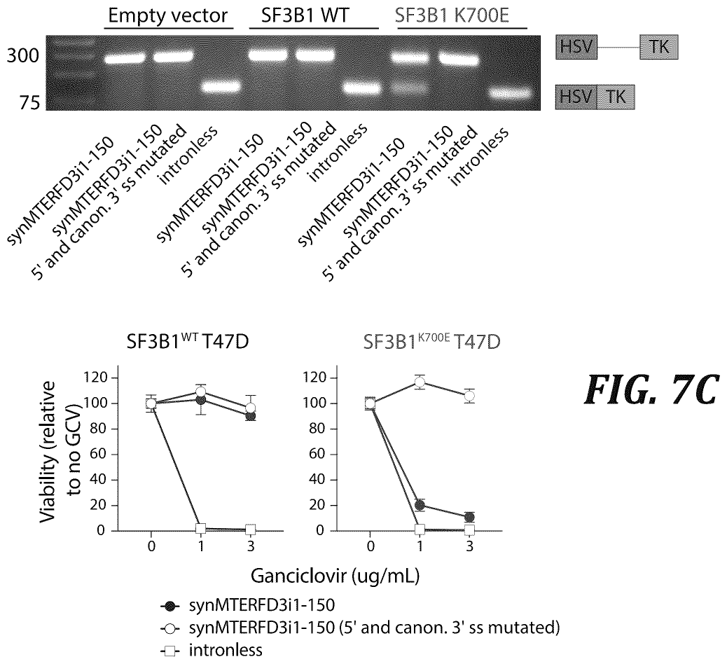

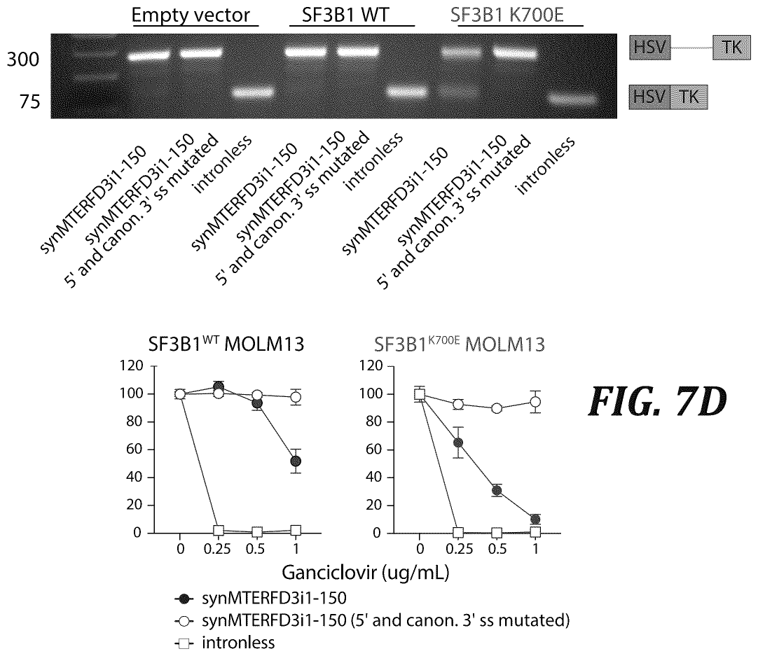

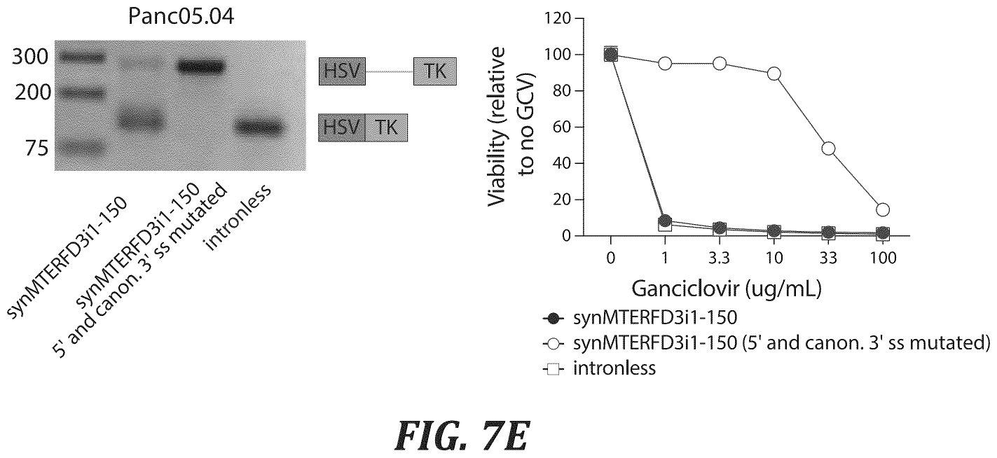

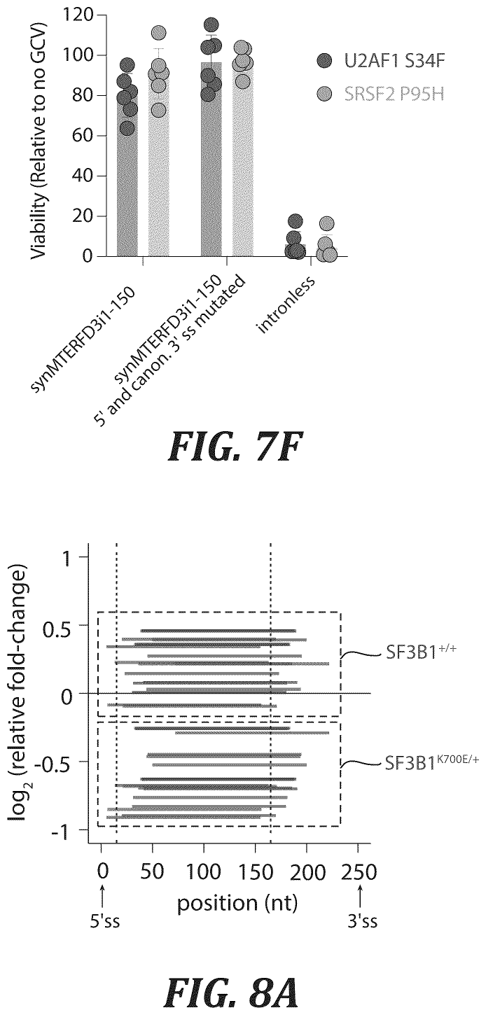

Mini-screen results were validated by introducing individual constructs from the mini-library into WT or SF3B1-mutant K562 cells and measuring relative depletion, confirming that the parallelized functional screen yielded accurate estimates of fitness costs following GCV treatment (FIG. 2G). The generalizability of this approach to other cell types was tested next. Isogenic MCF10A breast epithelial cells with (SF3B1K700E) or without an endogenous SF3B1 mutation, which recapitulated the expected mutation-driven mis-splicing of endogenous genes, were initially studied (FIGS. 7A and 7B). Expressing synthetic intron-containing HSV-TK and administering GCV resulted in strongly mutation-dependent death of MCF10A cells (FIG. 2H), confirming that the synthetic intron enables efficient targeting of multiple cell types. The synthetic intron was efficiently excised from HSV-TK pre-mRNA in SF3B1-mutant K562 and MCF10A cells, but not in SF3B1-wild-type K562 or MCF10A cells, confirming that GCV sensitivity arose from mutation-dependent splicing of the synthetic intron (FIG. 2I). Next, these studies were extended to additional cancer cell lines: T47D cells (breast cancer) and MOLM-13 cells (AML) engineered to transgenically express WT or mutant (K700E) SF3B1, as well as Panc05.04 cells (pancreatic cancer) bearing endogenous SF3B1Q699H/K700E. In each case, excision of the synthetic intron specifically occurred in SF3B1-mutant cells, and resulted in dose-dependent cell death upon GCV treatment (FIGS. 7C-7E). Intron excision and GCV-dependent cell death was specific to SF3B1 mutations and was not induced by the recurrent spliceosomal mutations SRSF2P95H or U2AF1S34F, consistent with initial results (FIGS. 7A and 7F).

Next, the approach was expanded to a massively parallel assay. Eight thousand, eight hundred and seventy eight distinct introns were designed to test the functional consequences of perturbing diverse features, including intron length, 5′ss and canonical 3′ss strengths, cryptic 3′ss position and multiplicity, pyrimidine and purine contents, branchpoint position and multiplicity, and nucleotide and dinucleotide identity (TABLE 2). Each intron was derived by applying defined changes to a “parent” synthetic intron from the mini-screen (synMTERFD3i1-250, synMTERFD3i1-150, or synMTERFD3i1-100). These 8,878 introns were synthesized as an oligonucleotide array, which was cloned into HSV-TK. K562 cells were infected and entire introns from genomic DNA was sequenced to estimate how each affected cell viability upon GCV administration.

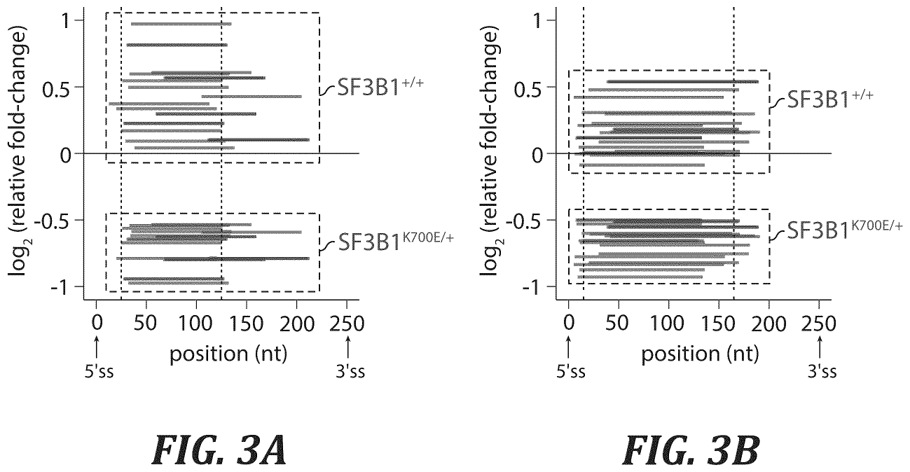

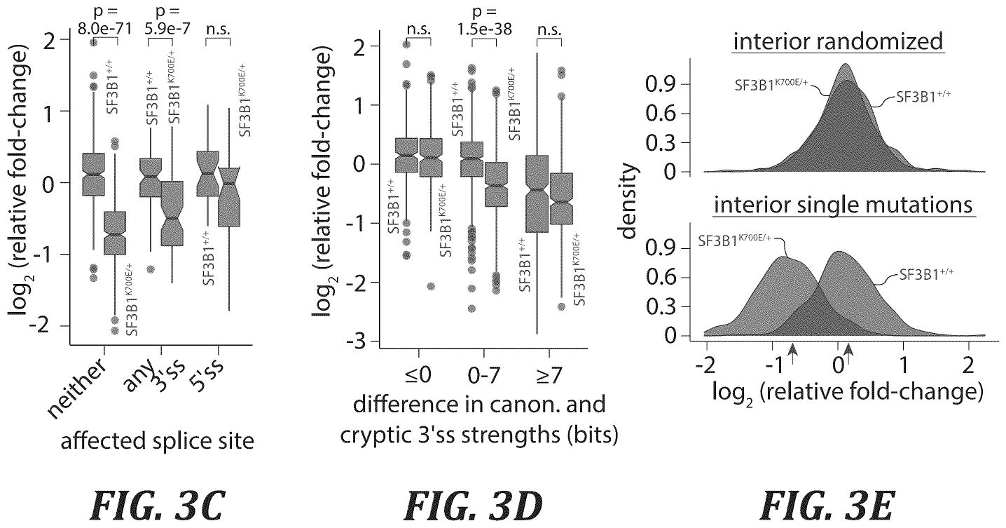

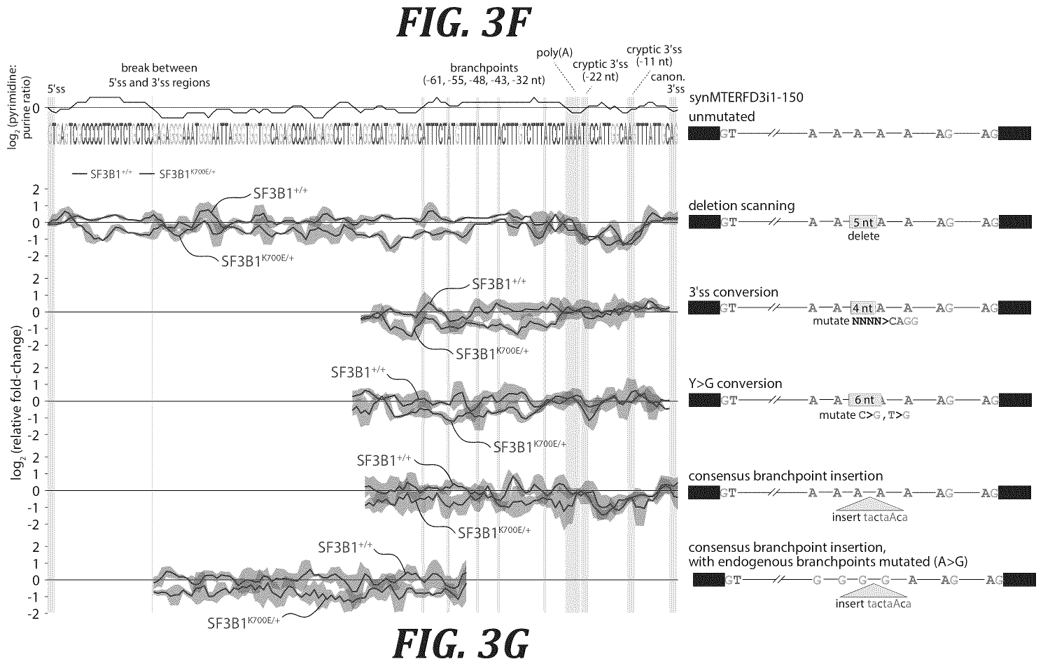

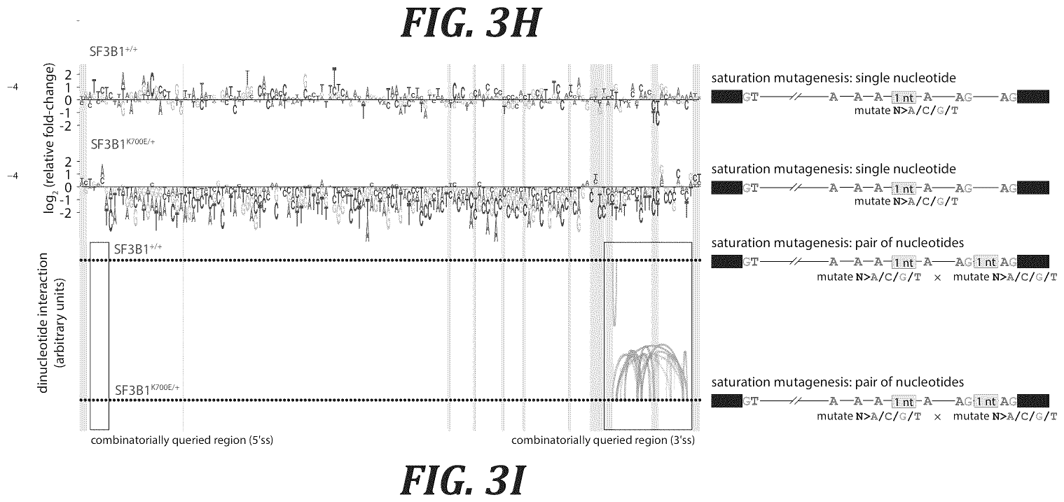

The resulting data illuminated global features governing mutation responsiveness. Iteratively deleting each consecutive 100 nt of the synMTERFD3i1-250 intron revealed that shortening the original 250 nt synthetic intron to 150 nt while maintaining mutation responsiveness required preserving the first 25 and last 125 nt (FIG. 3A). Shortening to 100 nt required preserving the first 15 and last 85 nt, although as in the mini-screen, 100 nt introns exhibited modestly reduced mutation responsiveness relative to 150 nt introns. Extreme shortening to 75 nt was possible, although with further reduced mutation responsiveness (FIG. 3B). These unbiased data indicate that the rationally designed deletions used to construct synMTERFD3i1-150 and synMTERFD3i1-100 were surprisingly close to optimal.

Both canonical and cryptic splice sites were critical for mutation-dependent behavior, with significant perturbations not tolerated. On average, constructs with single-nucleotide mutations within 10 nt of the 5′ss were rarely depleted in either genotype, suggesting that the strong, consensus nature of the 5′ss is required for intron recognition. Constructs with single-nucleotide mutations near any 3′ss (within the last 26 nt of the intron) had reduced mutation responsiveness relative to unperturbed introns. In contrast, single-nucleotide mutations distal to any splice site frequently maintained responsiveness (FIG. 3C). Mutation responsiveness required keeping the canonical 3′ss modestly stronger than the most intron-distal cryptic 3′ss; splice site strengths could be shifted as long as this imbalance was maintained, but not exaggerated (FIG. 3D). Although the splice sites themselves were particularly critical, more interior intronic features were also important. Although single-nucleotide mutations within the intron interior (>10 nt from the 5′ss and >30 nt from the canonical 3′ss) were generally tolerated, randomly shuffling all nucleotides within this region was not: such interior-randomized constructs typically exhibiting no depletion in any genotype (FIG. 3E).

The massively parallel assay enabled high-resolution insight into critical sequence features (FIGS. 3F and 3G). Deletion scanning with windows ranging from 5-50 nt revealed that loss of either cryptic 3′ss caused genotype-independent depletion, while most deletions affecting the thymine-rich, branchpoint-containing region or adjacent poly(A) sequence abolished depletion for both genotypes. In contrast, the purine-rich region upstream of those features was largely dispensable. Sliding creation of an additional cryptic 3′ss or conversion of pyrimidine-rich sequence to purines generally maintained mutation responsiveness, as long as the critical ˜30 nt upstream of the canonical 3′ss were preserved. In contrast, inserting a consensus branchpoint sequence typically reduced mutation responsiveness, with genotype-independent depletion resulting from insertion of a branchpoint in between the two cryptic 3′ss, unless this insertion was performed concordantly with ablation of all four commonly used, endogenous branchpoints. In that context, branchpoint insertion frequently maintained mutation responsiveness, even when the new branchpoint was located unusually deep within the intron.

Saturation mutagenesis revealed that the intron is remarkably robust to single-nucleotide mutations, with most constructs maintaining excellent mutation responsiveness (FIG. 3H). The 3′ss were notable exceptions. Mutations affecting the AG dinucleotide of the canonical 3′ss prevented depletion in SF3B1-mutant cells, as did purine mutations at the −3 position. The cryptic 3′ss at position −11 was similarly important, with mutations affecting the AG dinucleotide strongly depleted in both WT and SF3B1-mutant cells. Several positions proved unexpectedly important in SF3B1-mutant cells, including +6 of the 5′ss and −6 and −10 of the canonical 3′ss. Mutations of many positions to adenine within the pyrimidine-rich, branchpoint-containing region were associated with strong depletion, while mutations that ablated branchpoints within this region preserved mutation responsiveness, confirming that variable branchpoint multiplicity is tolerated.

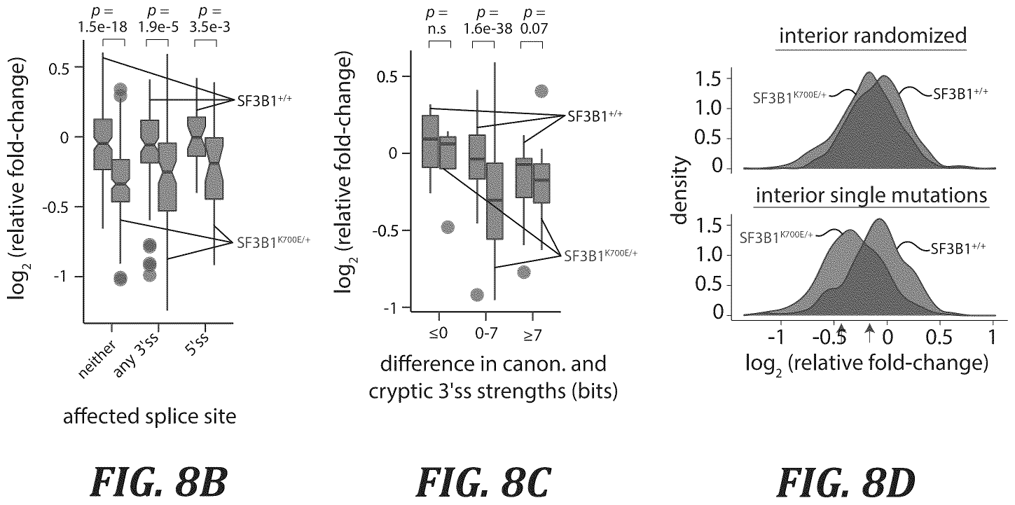

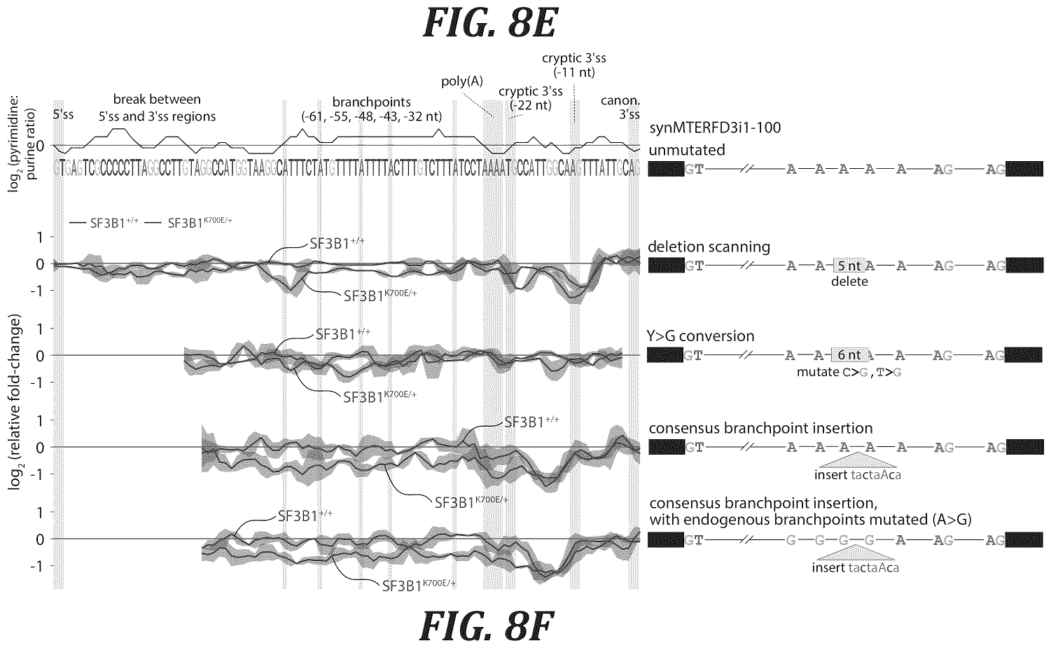

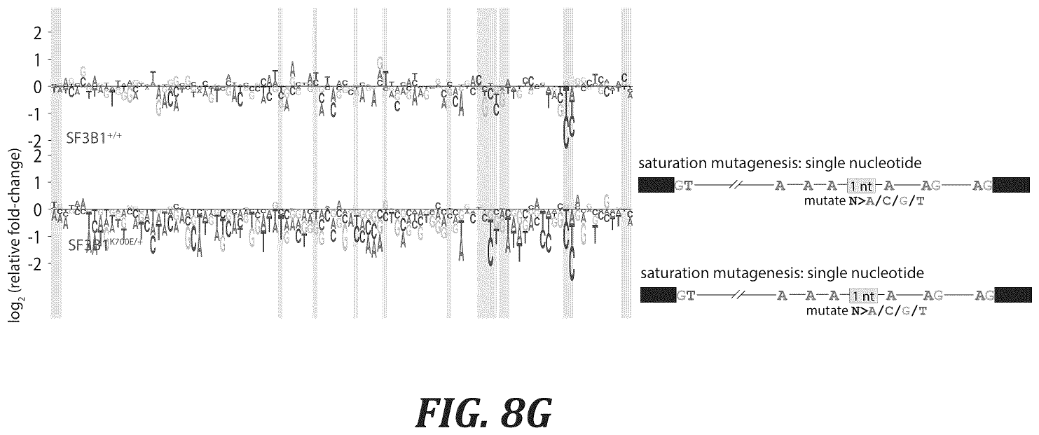

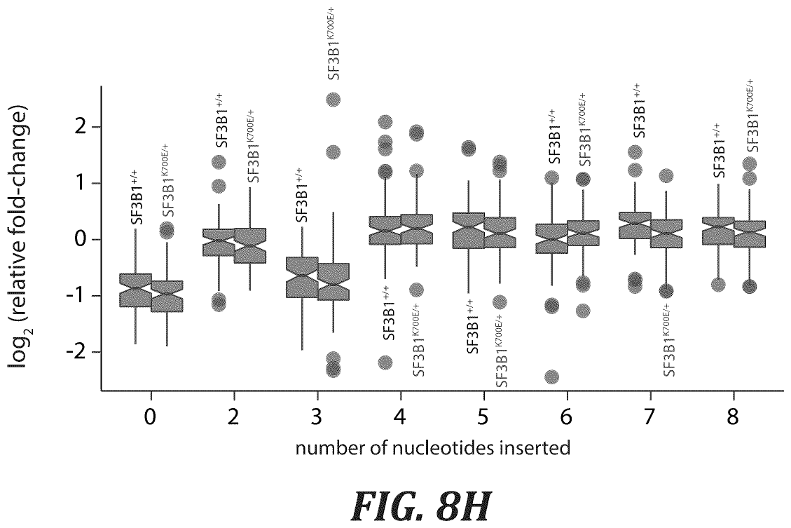

These and other observations were generally highly similar when the same modifications were applied to synMTERFD3i1-100, indicating that most critical sequence elements are independent of intron length (FIG. 8A-8G). Almost all variants of this very short intron exhibited more modest mutation-dependent splicing than did corresponding synMTERFD3i1-150-derived variants. Simultaneously inserting a strong polypyrimidine tract and 3′ss immediately upstream of the canonical 3′ss and one or more consensus branchpoints further upstream resulted in genotype-independent depletion, as expected (FIG. 3H). However, this depletion was still frequently more modest than that observed for synMTERFD3i1-150-derived variants, suggesting that overly short introns may not be efficiently spliced even when they have consensus splice sites. Depletion was similar when one to four consensus branchpoints were inserted, suggesting that increasing branchpoint multiplicity has few effects on splicing when a consensus branchpoint is present. This same insertion scanning revealed genotype-independent depletion when an extremely strong polypyrimidine tract and 3′ss was inserted 3 nt upstream of the canonical 3′ss—but not at any other position-suggesting that HSV-TK is permissive to insertion of a single glutamine residue at the exon-exon junction that was created (FIG. 8H).

Finally, a search was conducted for possible epistatic interactions within the critical 4 and 20 nt of the 5′ss and 3′ss regions. Saturation mutagenesis was performed of all 54 and 1,710 nucleotide pairs that did not disrupt the GT or AG of the 5′ss and canonical 3′ss and searched for enrichment or depletion exceeding that expected based on single-nucleotide mutagenesis by ≥2-fold (FIG. 3I). For WT cells, only a single interaction at the 3′ss met this threshold: the G and following nucleotide of the unusual TG cryptic 3′ss at −22 nt. SF3B1-mutant cells exhibited more complex interactions, particularly for positions between the two cryptic 3′ss, reinforcing the complex and essential nature of this region. No epistatic interactions met the threshold at the 5′ss for either genotype.

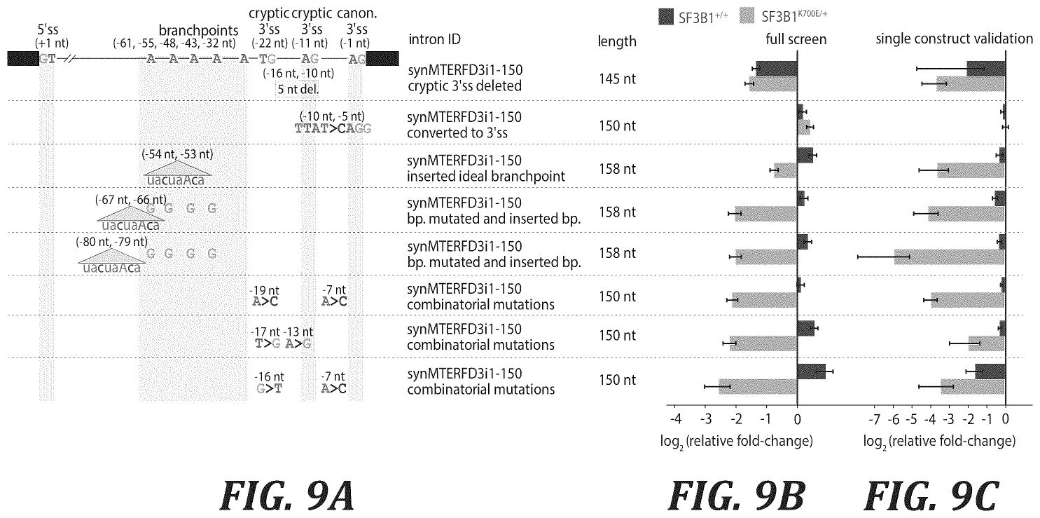

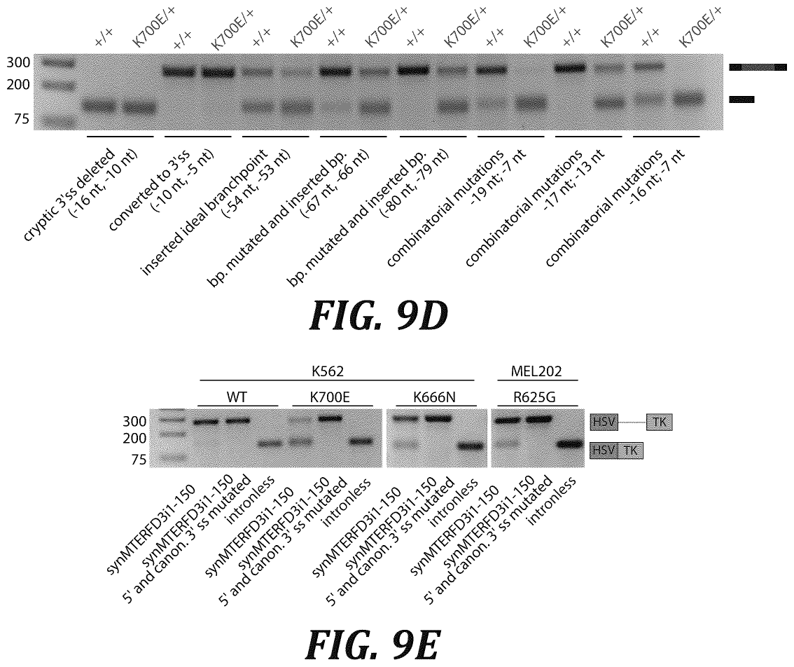

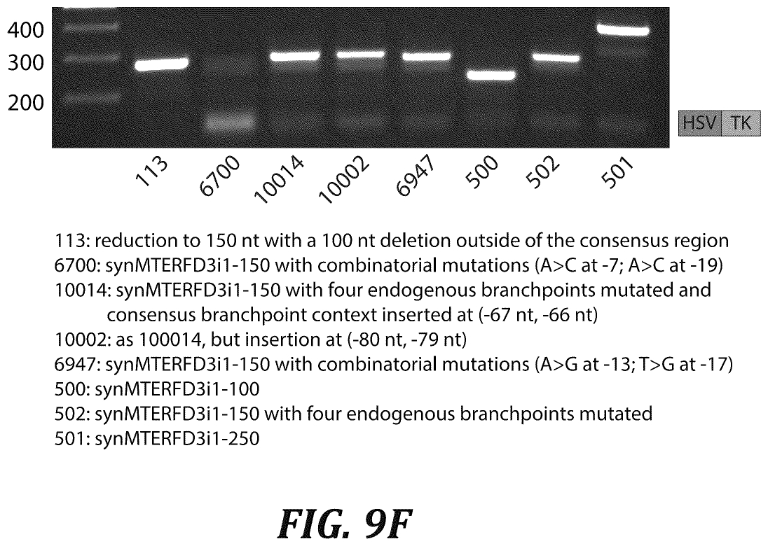

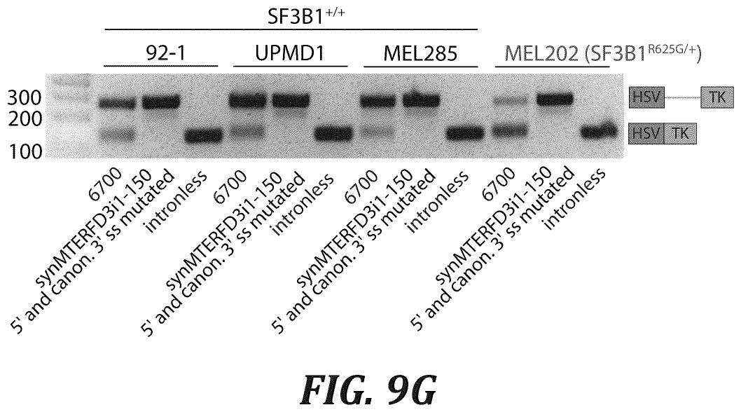

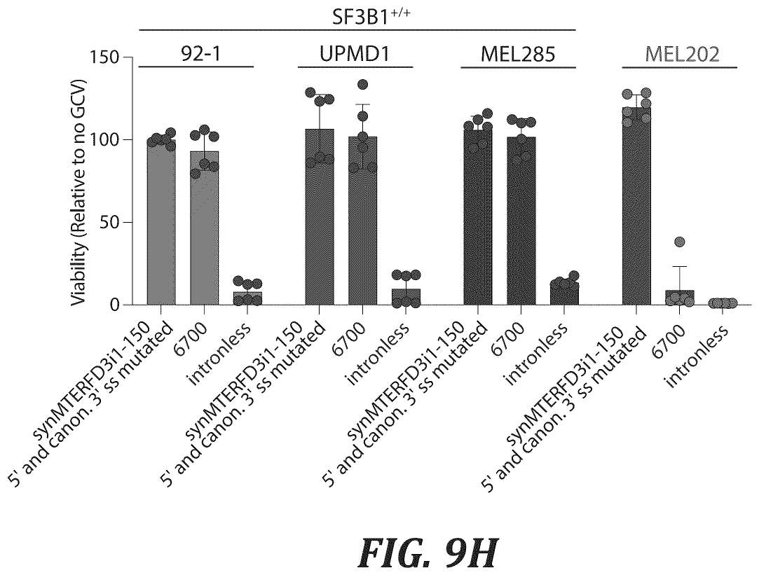

Eight synMTERFD3i1-150-derived variants representing different modification classes were selected for single-construct study. These studies confirmed the essentiality of the immediate sequence context of the cryptic 3′ss at −11 nt; demonstrated that variants with far-distal consensus branchpoints gave rise to enhanced splicing in SF3B1-mutant cells; and indicated that select combinatorial mutations fully eliminated splicing in WT cells (FIGS. 9A-9D). Testing these constructs in uveal melanoma cells confirmed that synMTERFD3i1-150 was excised in the context of endogenous SF3B1R625G, although less efficiently than for SF3B1K700E (the most common SF3B1 mutation and the focus of most of our above studies). However, efficient splicing in the context of SF3B1R625G was restored by introducing a combinatorial mutation (A>C at −7 nt; A>C at −19 nt) into synMTERFD3i1-150 (FIGS. 9E-9G). HSV-TK interrupted by this synthetic intron, which was nominated by the screen as a promising candidate, drove mutant SF3B1-dependent cell death when introduced into uveal melanoma cell lines with or without SF3B1 mutations (FIG. 9H).

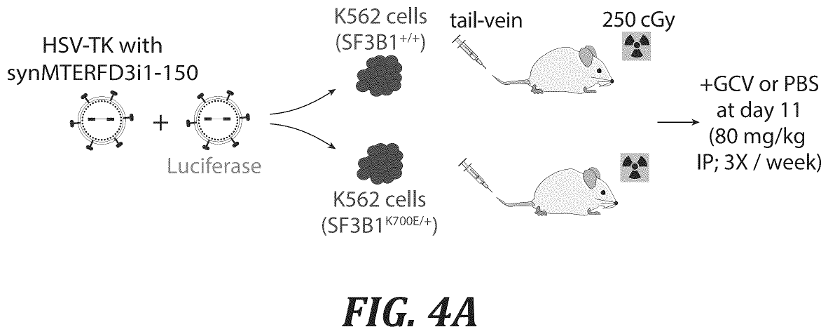

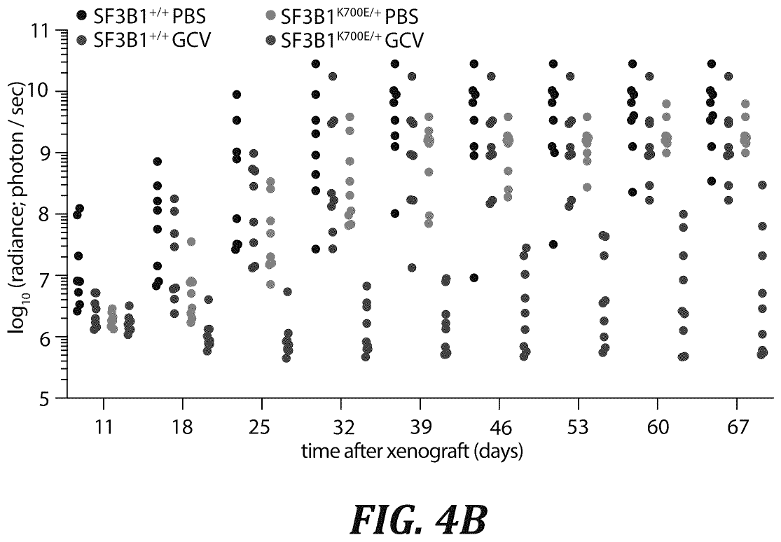

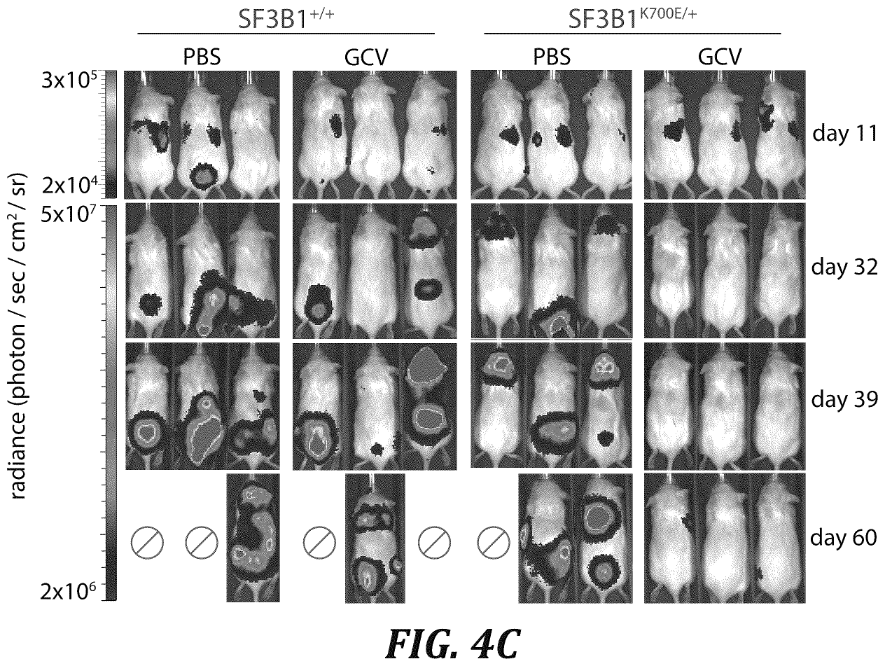

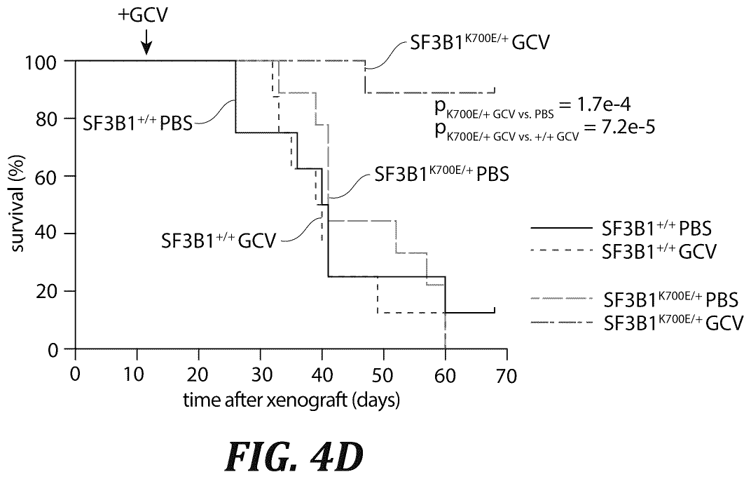

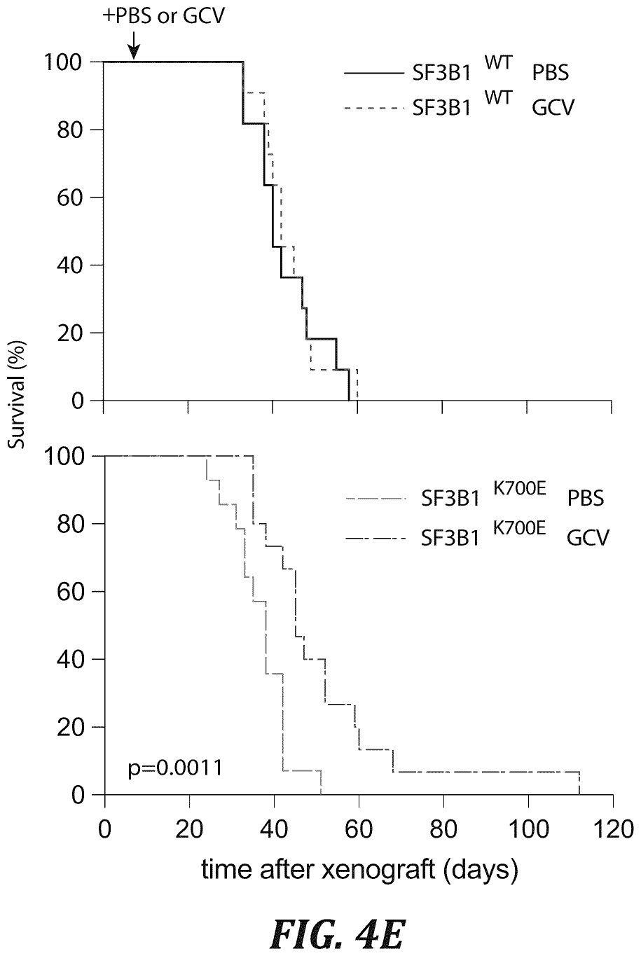

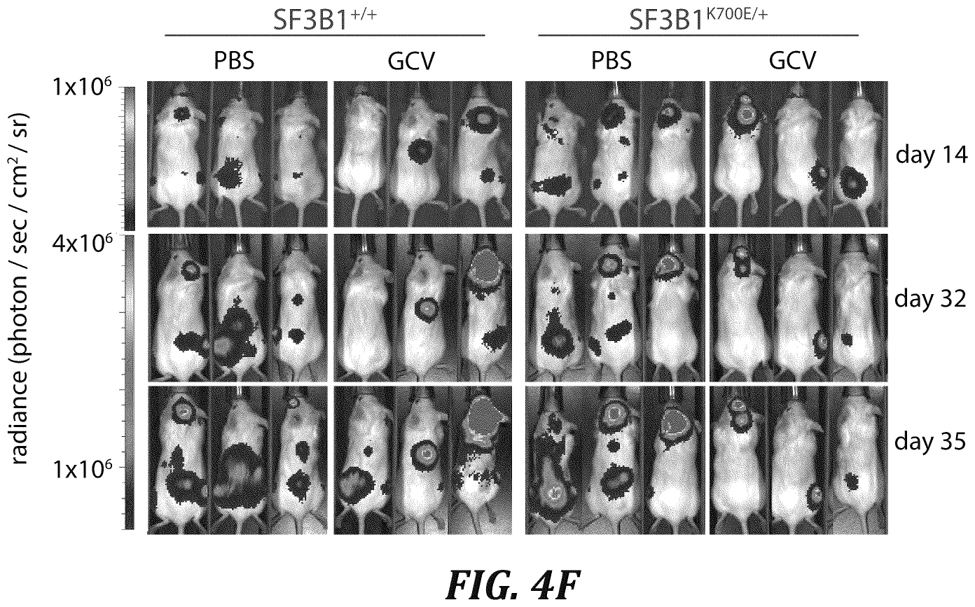

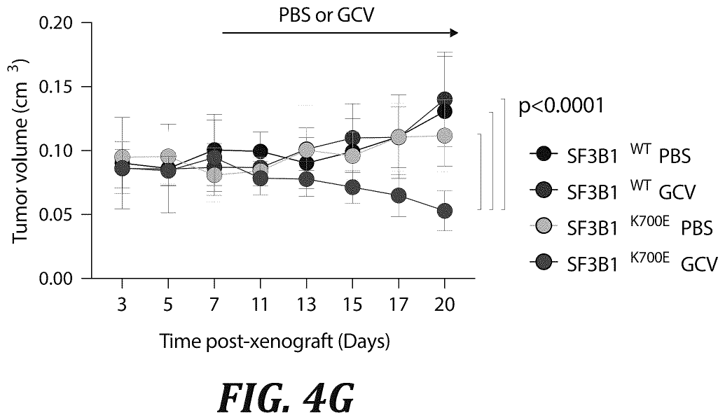

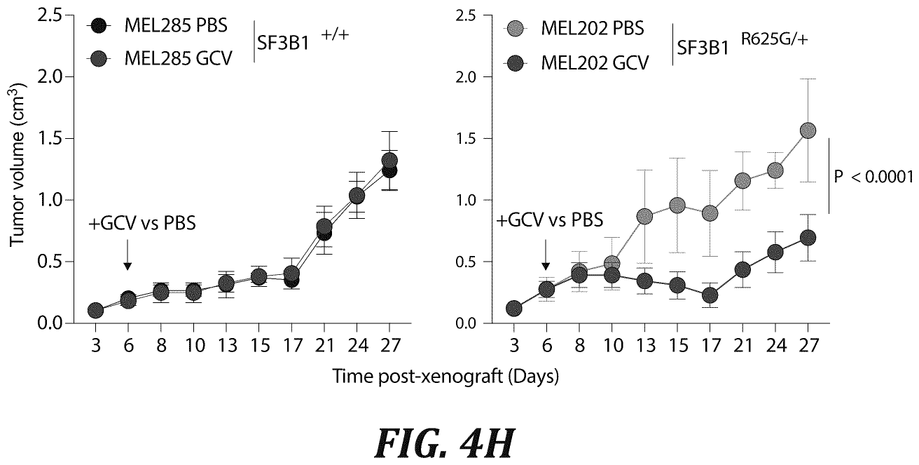

Having identified the key features governing mutation responsiveness, the next step was to test whether synthetic introns permitted mutation-dependent cancer cell killing in vivo. A focus was made on synMTERFD3i1-150 for these experiments, as it served as the parent synthetic intron from which most of the full library was derived. Luciferase-GFP constructs were introduced into WT or SF3B1-mutant K562 cells expressing HSV-TK interrupted by synMTERFD3i1-150, tail vein injections of these cells were performed into NOD-scid IL2Rgnull (NSG) mice. The mice were treated with PBS or GCV and monitored for leukemia burdens with live imaging (FIG. 4A). Both genotypes formed aggressive leukemias which rapidly resulted in lethality for PBS-treated animals, independent of genotype. GCV administration, in contrast, drove immediate and sustained suppression of SF3B1-mutant leukemic burden, with no effects on WT leukemias (FIGS. 4B and 4C). GCV treatment resulted in correspondingly significantly increased survival (p=1.7e-4 for GCV- vs. PBS-treated SF3B1-mutant leukemias). Only one GCV-treated animal engrafted with an SF3B1-mutant leukemia had died at day 70 (although the immediate cause of death of this animal was unclear, as it had minimal leukemic burden by imaging or necropsy), at which point almost all animals in other treatment arms had died (FIG. 4D).

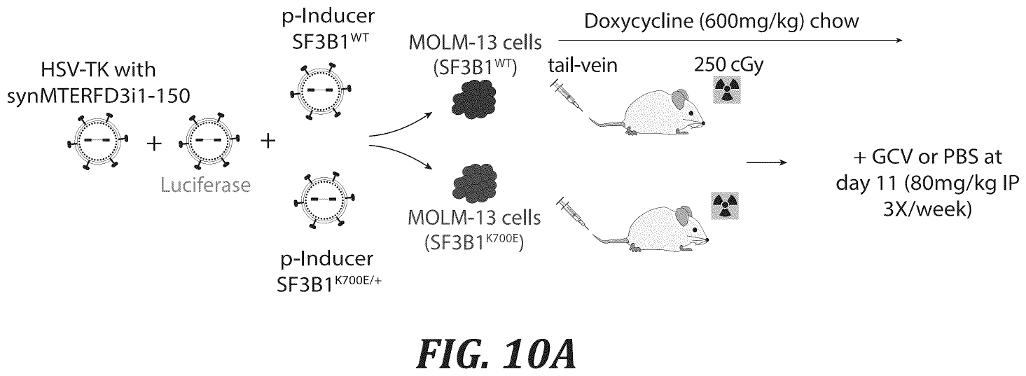

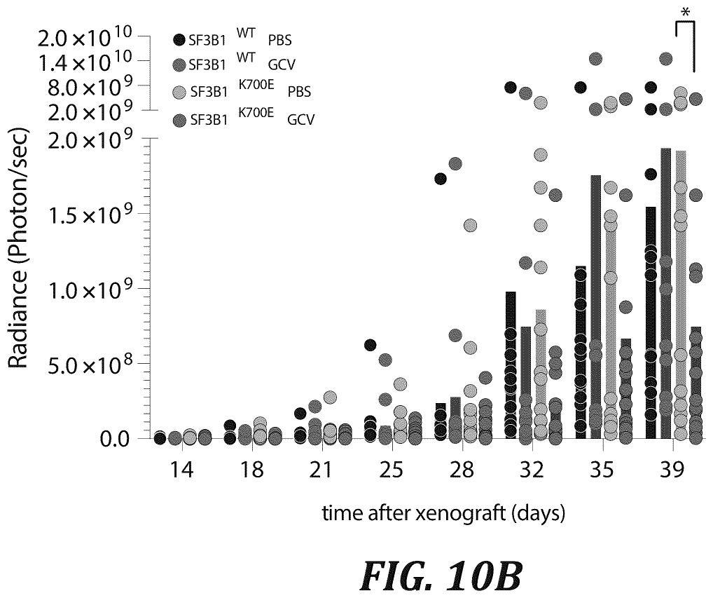

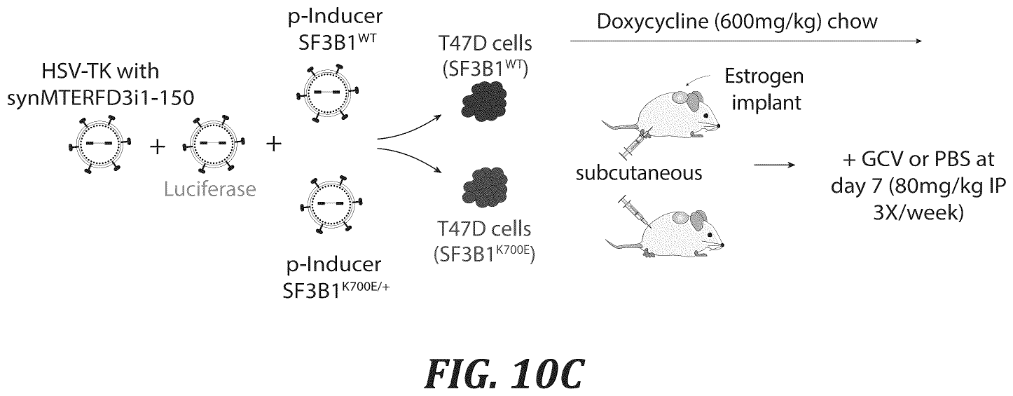

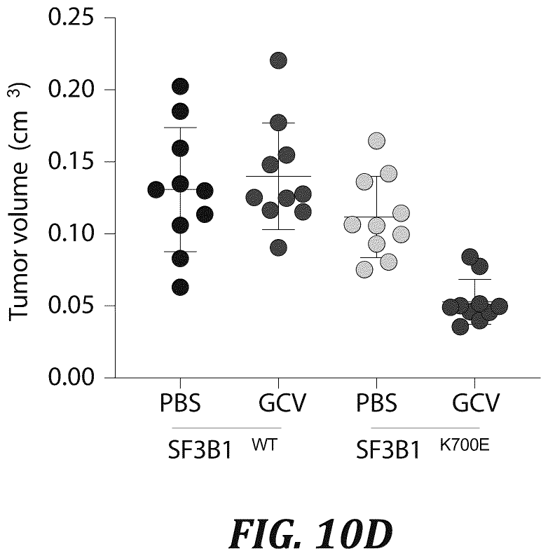

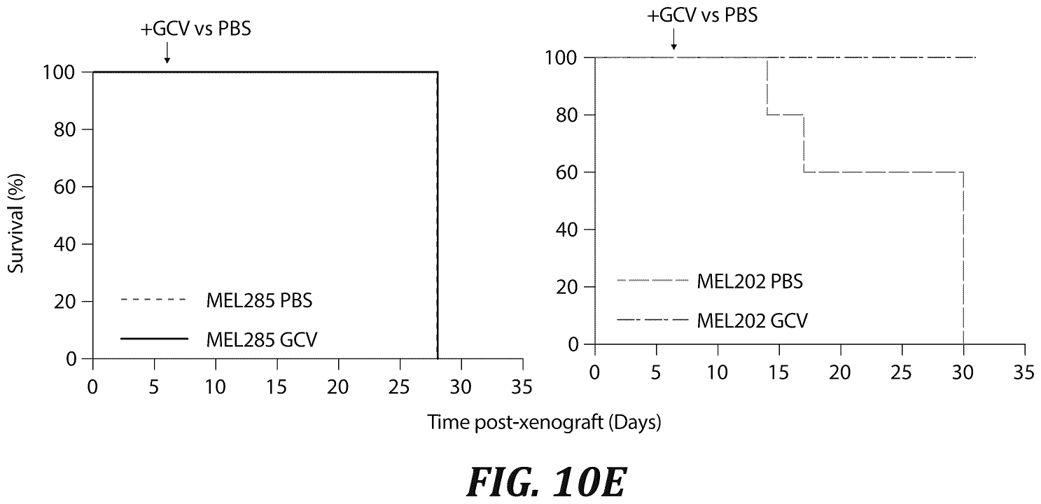

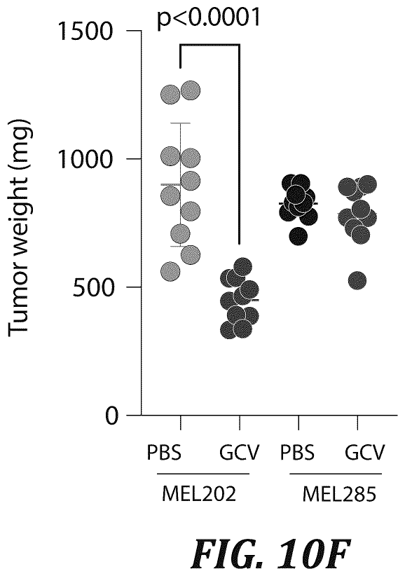

Next, the potential efficacy of using synthetic introns was tested in other cancer models. HSV-TK interrupted by synMTERFD3i1-150 was introduced into MOLM-13 cells (acute myeloid leukemia) engineered to permit doxycycline-inducible expression of SF3B1 WT or K700E and Luciferase imaging. These cells were then engrafted into NSG mice, which were treated with doxycycline and GCV. Leukemia burden and survival were monitored. As for the K562 model, significantly prolonged survival and reduced tumor burden were observed in the GCV-treated arm engrafted with SF3B1K700E-expressing MOLM-13 cells (FIGS. 4E, 4F, 10A, and 10B). A similarly structured experiment using subcutaneous engraftment of T47D (breast cancer) cells engineered to permit doxycycline-inducible expression of SF3B1 WT or K700E similarly revealed specifically reduced tumor burden in the GCV-treated arm engrafted with SF3B1K700E-expressing cells (FIGS. 4G, 10C, and 10D). Finally, HSV-TK interrupted by synMTERFD3i1-150 with A>C at −7 nt; A>C at −19 nt was introduced into uveal melanoma cells with (MEL202; SF3B1R625G) or without (MEL285) an SF3B1 mutation and these cells were subcutaneously engrafted into NSG mice. As expected, significantly reduced tumor burden and prolonged survival were observed specifically in the GCV-treated arm engrafted with SF3B1R625G-expressing cells (FIGS. 4H, 4I, 10E, and 10F).

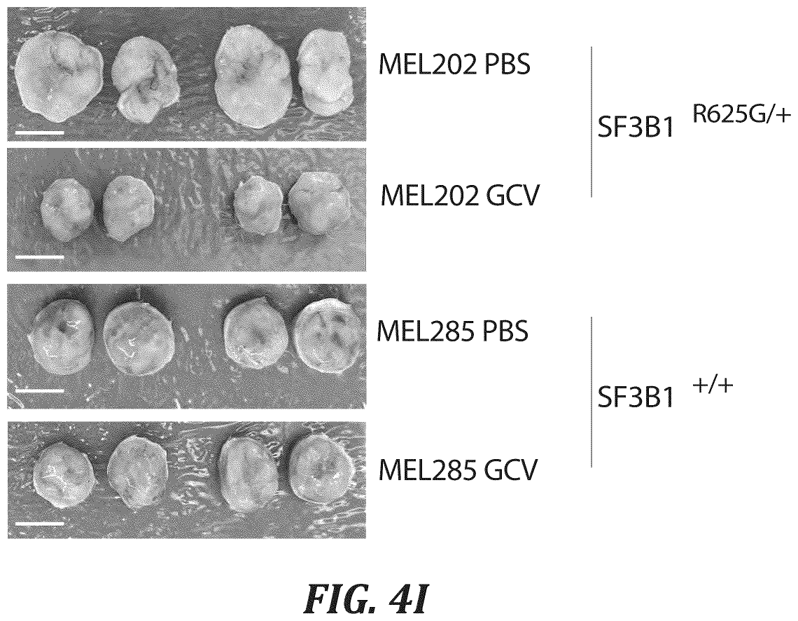

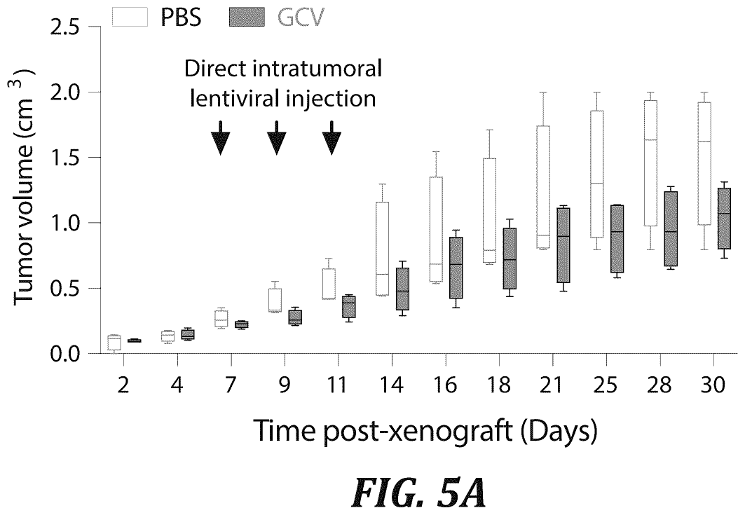

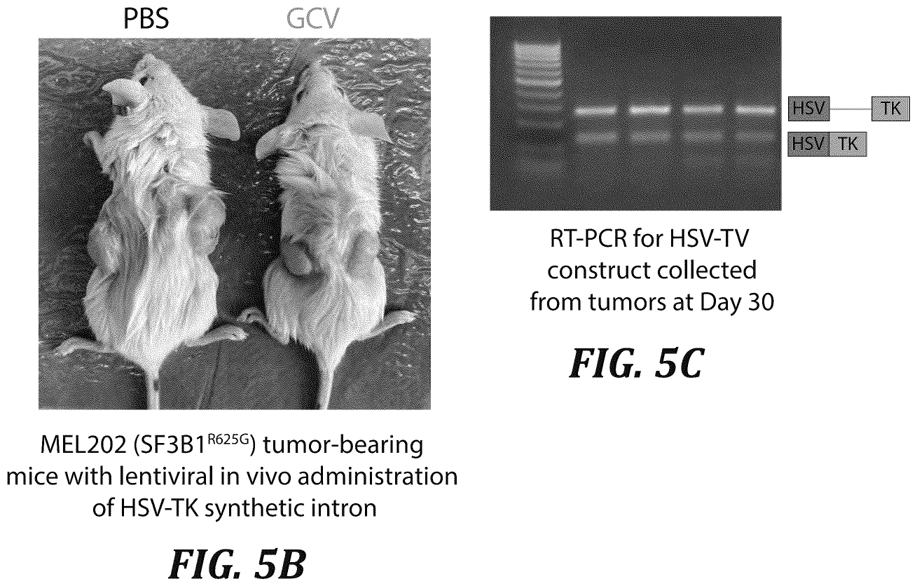

Although the above experiments clearly demonstrate that synthetic introns enable SF3B1 mutation-dependent targeting of diverse cancer types, they all utilized in vitro delivery of the synthetic intron-containing therapeutic construct. Therefore, it was tested whether synthetic intron-containing constructs could be delivered to established tumors in vivo. NSG mice were engrafted with uveal melanoma cells bearing endogenous SF3B1R625G and delivered HSV-TK interrupted by synMTERFD3i1-150 with A>C at −7 nt; A>C at −19 nt via direct intratumoral injection of high-titer lentivirus. This experiment revealed spliced HSV-TK expression in tumors that persisted weeks after the last lentiviral injection and corresponding suppression of tumor growth in the GCV-treated arm, demonstrating that delivery of synthetic intron-containing therapeutic constructs to established tumors is both possible and therapeutically efficacious (FIGS. 5A-5C).

This study demonstrates the feasibility and therapeutic potential of harnessing recurrent, pro-tumorigenic splicing alterations to engineer new molecular therapeutics. Synthetic introns have several attractive characteristics for further therapeutic development. First, they can be rationally designed to respond to defined, cancer-initiating mutations. Second, their small size facilitates delivery. Third, as their mechanism of action is post-transcriptional, they are not subject to constraints frequently imposed by transcriptional control methods of achieving cancer-specific expression, such as a comparatively weak promoter.

As SF3B1 mutations are common across diverse cancer types, synthetic introns may facilitate the development of pan-cancer gene therapies. Furthermore, because synthetic intron function exploits a fundamental property of SF3B1 mutations from which their pro-oncogenic activity arises, resistance to mutation-dependent splicing may be unlikely to develop. Synthetic introns will thereby complement other synthetic biology-based methods for targeted protein expression in response to molecular signals (e.g., Lienert, F., et al. Synthetic biology in mammalian cells: next generation research tools and therapeutics. Nat Rev Mol Cell Bio 15, 95-107 (2014); Wu, M.-R., Jusiak, B. & Lu, T. K. Engineering advanced cancer therapies with synthetic biology. Nat Rev Cancer 19, 187-195 (2019), each of which is incorporated herein by reference in its entirety), including splicing-based devices that utilize RNA aptamers to sense NF-κB and Wnt signaling (Culler, S. J., Hoff, K. G. & Smolke, C. D. Reprogramming cellular behavior with RNA controllers responsive to endogenous proteins. Science (New York, NY) 330, 1251-1255 (2010), incorporated herein by reference in its entirety) and protease-based devices that sense pro-oncogenic ErbB receptor activity (Chung, H. K. et al. A compact synthetic pathway rewires cancer signaling to therapeutic effector release. Science 364, eaat6982 (2019), incorporated herein by reference in its entirety).

The disclosed synthetic introns are expected to be widely applicable beyond the HSV-TK system. For example, synthetic introns could be used to achieve mutation-dependent expression of other proteins with anti-cancer potential, such as cytokines, chemokines, and cell-surface proteins (Nissim, L. et al. Synthetic RNA-Based Immunomodulatory Gene Circuits for Cancer Immunotherapy. Cell 171, 1138-1150.e15 (2017), incorporated herein by reference in its entirety). Importantly, as synthetic introns yield mutation-dependent splicing and protein expression, delivery of a synthetic intron-bearing therapeutic vector to healthy cells is expected to have negligible consequences. The disclosed synthetic intron-containing fluorescent reporters (FIGS. 1A-1G) could be used to screen for genes and compounds which suppress cancer-specific alterations in RNA splicing. Finally, this study illustrates the power of massively parallel assays for functional interrogation of splicing, including the derivation of rational rules governing mutation-dependent splicing that will facilitate the future design and improvement of these and other synthetic introns.

### Methods

Expression Vector Cloning

mCardinal-pBiCMV-mEmerald: Oligonucleotides containing the endogenous GAPDH 5′ UTR (from genomic DNA), mEmerald coding sequence (from mEmerald-N1; Addgene Plasmid 53976), and mCardinal coding sequence (from mCardinal-N1; Addgene Plasmid 54590) were synthesized and cloned into the pRRLSIN.cPPT.PGK-GFP.WPRE (Addgene Plasmid 12252) backbone, along with the pBiCMV promoter (from pBi-CMV1 (Clontech); Addgene Vector 6166), to replace the PGK-GFP sequence. The GAPDH 5′ UTR sequence is set forth in SEQ ID NO:53. Orientation of fragments was as illustrated in FIG. 1E. hPGK-HSV-TK-P2A-mCherry: The HSV-TK coding sequence (from pAL119-TK; Addgene Plasmid 21911) was cloned into the pRRLSIN.cPPT.PGK-SF3B1 WT-FLAG-P2A-mCherry.WPRE backbone (Pangallo, J. et al. Rare and private spliceosomal gene mutations drive partial, complete, and dual phenocopies of hotspot alterations. Blood (2020) doi:10.1182/blood.2019002894, incorporated herein by reference in its entirety) to replace the SF3B1 WT-FLAG sequence. The HPGK-HSV-TK-P2A-mCherry sequence was flipped using XhoI and SalI enzymes so that the intron is not spliced out during lentivirus production. hPGK-PuroR-P2A-HSV-TK: The puromycin resistance coding sequence (from pLenti CMV GFP Puro; Addgene Plasmid 17448) with P2A was cloned into the hPGK-HSV-TK-P2A-mCherry backbone after excising the P2A-mCherry sequence. PCR primers for cloning are specified in TABLE 3. All pieces were amplified with Phusion or Q5 polymerase (New England Biolabs). Assembly was performed with NEBuilder HiFi (New England Biolabs) according to the manufacturer's instructions. All truncated intron sequences were initially synthesized as gBlocks (Integrated DNA Technologies).

Transfection and Flow Cytometry

K562 cells were transfected with fluorescent reporters using a Lonza Cell Line Nucleofector V Kit as described in the kit protocol. Cell were spun down and resuspended in PBS 72 hours after transfection, after which flow cytometry was performed using the GFP and APC wavelengths. Gates were first set to capture all live cells, then set to only analyze mCardinal⁺ cells, after which mEmerald/mCardinal was computed for each cell.

Lentivirus Production

Expression vector plasmids were co-transfected with psPAX2 (Addgene plasmid 12260) and envelope vector pMD2.G (Addgene plasmid 12259) into 293T cells. Lentivirus was collected from the supernatant 48 hours after transfection. Stable cell lines were made by transducing K562 or MCF10A cells with lentivirus at multiplicities of infection (MOIs) of 1 (FIGS. 2C, 2G, and 2H), 0.3 (mini-library), and 0.1 (full library). Positive integrants were selected by treating with puromycin (hPGK-PuroR-P2A-HSV-TK) or flow sorting for mCherry (hPGK-HSV-TK-P2A-mCherry).

Cell Viability Measurements

Cell viability was measured for single-construct experiments in cell culture by the CellTiter-Glo Luminescent Cell Viability Assay (Promega). For FIG. 2C, K562 cells expressing HSV-TK with the indicated synthetic introns were seeded at a density of 10,000 cells/100 μL/well in a 96-well plate in biological triplicate, and then treated with 0-100 μg/mL GCV or untreated (negative control). Viability measured after 3 days of treatment. For FIG. 2G, K562 cells expressing HSV-TK with the indicated synthetic introns were seeded at a density of 5,000 cells/100 μL/well in 96-well plate in biological triplicate, and then treated with 100 μg/mL GCV or PBS (negative control). Viability was measured after 11 days of treatment.

RT-PCR to Study Splicing of Endogenous and Synthetic Introns

Total RNA was extracted using Direct-zol RNA Miniprep (Zymo Research). cDNA was synthesized using Superscript IV Reverse Transcriptase (Thermo Fisher Scientific) using the manufacturer's protocol. Gene-specific primers used for amplifications are listed in TABLE 3. Amplicons were analyzed using agarose gel electrophoresis and quantified using ImageJ (Fiji). Branchpoints were identified from lariat-spanning sequences as previously described (Pineda, J. M. B. & Bradley, R. K. Most human introns are recognized via multiple and tissue-specific branchpoints. Genes & development 32, 577-591 (2018), incorporated herein by reference in its entirety).

Cell Culture

Isogenic K562, NALM-6, and MCF10A cells with and without defined SF3B1 mutations were generated by Horizon Discovery as previously described (Inoue, D. et al. Spliceosomal disruption of the non-canonical BAF complex in cancer. Nature (2019); and Liu, B. et al. Mutant SF3B1 promotes AKT and NF-kB driven mammary tumorigenesis. J Clin Invest (2020) doi:10.1172/jci138315, each of which is incorporated herein by reference in its entirety). MEL202 and MEL270 cells were a gift from Boris Bastian (Griewank, K. G. et al. Genetic and molecular characterization of uveal melanoma cell lines. Pigment cell & melanoma research 25, 182-187 (2012), incorporated herein by reference in its entirety). K562 cells were grown at 37 C and 5% atmospheric CO₂ in Iscove's Modified Dulbecco's Medium (IMDM; Gibco) supplemented with 10% fetal bovine serum (Gibco). NALM-6, MEL270, and MEL202 cells were grown in RPMI supplemented with 10% fetal bovine serum (Gibco) and 1% penicillin/streptomycin. MEL202 cells were additionally supplemented with 1% (2 mM) GlutaMAX (Gibco).

K562 Xenografts

Luciferase-expressing K562 cells were established by infecting cells with lentivirus created from pMSCV-Luciferase-PGK-GFP (Addgene plasmid 18782; HygR replaced by GFP) at MOIs of 0.9 (SF3B1^{+/+}) and 0.5 (SF3B1^{+/K700E}). GFP⁺ cells were isolated by flow sorting 7 days after infection. K562 cells expressing Luciferase and HSV-TK interrupted by synMTERFD3i1-150 were intravenously injected into sub-lethally irradiated (250 cGy) NOD-scid IL2Rgnull (NSG) mice (2 million cells/mouse). Leukemic cells were allowed to grow for 11 days before mice were treated with PBS (negative control) or ganciclovir (GCV; 80 mg/kg) via intraperitoneal (IP) injection three times per week. Bioluminescence imaging was carried out weekly with 150 mg/kg of D-Luciferin.

Animal Use

All animal procedures were conducted in accordance with the Guidelines for the Care and Use of Laboratory Animals and approved by the Institutional Animal Care and Use Committees at Memorial Sloan Kettering Cancer Center. NSG mice were obtained from the Jackson Laboratory.

Mini-Library Construction, Screen, and Analysis

Each synthetic intron used in the mini-library was ordered individually as a gBlock (Integrated DNA Technologies), consisting of the desired intron flanked by homology arms for cloning (5′ arm: TCGACCAGGGTGAGATATCGGCCGG (SEQ ID NO:158); 3′ arm: GGACGCGGCGGTGGTAATGACAAGC (SEQ ID NO:159; TABLE 3). The gBlocks were then mixed in equal proportions before being cloned into hPGK-PuroR-P2A-HSV-TK using a previously published strategy for pooled cloning (Thomas, J. D. et al. RNA isoform screens uncover the essentiality and tumor-suppressor activity of ultraconserved poison exons. Nature genetics 52, 84-94 (2020), incorporated herein by reference in its entirety). This intron mix was then amplified using NEBNext High Fidelity Ready Mix (New England Biolabs) and purified using 1.8×AMPure XP SPRI beads (Beckman Coulter). The backbone for the library was amplified using Q5 polymerase (New England Biolabs). The library was transformed and amplified using Endura ElectroCompetent Cells (Lucigen, 60242-2) and large LB plates. The library was maxiprepped using a Macherey-Nagel MaxiPrep kit (Thermo Fisher Scientific, Cat 740414.10).

WT or SF3B1-mutant K562 cells were infected with lentivirus encoding the mini-library at an MOI of 0.3 and untreated or treated with GCV (100 ug/mL) for 6 days. Genomic DNA was collected at day 6 and the resulting Illumina libraries were sequenced with 2×150 bp reads (Illumina MiSeq). Depletion/enrichment of each construct was estimated as follows. For each sample, reads were normalized to the total reads mapped. The relative fraction of reads mapping to each intron was then estimated by dividing the numbers of normalized reads mapped to an intron by the total reads mapped in the sample. The standard deviation was calculated using the sample proportion P for each intron (σ=sqrt [P(1−P)/n]). A fold-change was calculated for each intron by dividing the proportion of the intron in the treated GCV samples by the fraction of the intron in the untreated samples. Error propagation was used to estimate the standard deviation. The final relative fold-changes were computed by normalizing fold-changes such that the fold-change of synMTERFD3i1-250 in the pilot screen was identical to the experimentally measured fold-change in a single-construct experiment (FIG. 2C, 100 μg/mL of GCV).

Full Library Construction, Screen, and Analysis

Introns constituting the full library were synthesized as an oligonucleotide array (Twist Bioscience). Each oligonucleotide consisted of a desired intron flanked by homology arms for cloning, where the homology arms consisted of the 3′ end of the first HSV-TK exon and the 5′ end of the second HSV-TK exon. The homology arms for each intron were selected such that the final oligonucleotide was 200 nt long, so that each homology arm had length ((200 nt−intron length)/2). 10 ng of the library was amplified using primers cattgttatctgggcgcttgtcattaccaccgccgcgtcc (SEQ ID NO:140) and ccacacaacaccgcctcgaccagggtgagatatcggccgg (SEQ ID NO:141) (TABLE 3) using NEBNext Master Mix (New England Biolabs) for 2 cycles at 63° C. and 10 cycles at 72° C. (for a total of 12 cycles); this amplification resulted in homology arms of consistent lengths across the whole library. After amplification, the library was cleaned up with a 1.8×SPRI bead cleanup (Beckman Coulter). The backbone was separately amplified using NEBNext Master Mix (New England Biolabs) with primers ggacgcggcggtggtaatgacaagcgcccagataacaatg (SEQ ID NO:138) and ccggccgatatctcaccctggtcgaggcggtgttgtgtgg (SEQ ID NO:139) (TABLE 3) using a two-step PCR (annealing and extension steps were combined into one step at 72° C.). The amplified library and backbone were assembled using NEBuilder HiFi (New England Biolabs) in 8 identical separate reactions, each incubated for an hour and then cleaned up with a 0.8× SPRI bead cleanup (Beckman Coulter). The insert to backbone ratio was 5:1. The resulting library was transformed and amplified using Endura ElectroCompetent Cells (Lucigen, 60242-2) and large LB plates. The library was maxiprepped using a Macherey-Nagel MaxiPrep kit (Thermo Fisher Scientific, Cat 740414.10).

WT or SF3B1-mutant K562 cells were infected with lentivirus encoding the full library at an MOI of 0.1 and treated with GCV (100 ug/mL) for 8 days. Genomic DNA was collected at day 0 and day 8 and the resulting Illumina libraries (triplicates) were sequenced with both 2×150 bp and 2×250 bp reads (Illumina MiSeq).

After sequencing, reads were first trimmed using cutadapt v2.1 (Martin, M. Cutadapt removes adapter sequences from high-throughput sequencing reads. Embnet J 17, 10-12 (2011), incorporated herein by reference in its entirety) to remove sequenced Illumina adapters (acgcggcggtggtaatgacaa (SEQ ID NO:54) for the 3′ end and ggccgatatctcaccctggtc (SEQ ID NO:55) for the 5′ end), and additionally remove sequences corresponding to portions of the HSV-TK cDNA. Each pair of trimmed reads was then combined into a single read using FLASH v1.2.11 (Magoč, T. & Salzberg, S. L. FLASH: fast length adjustment of short reads to improve genome assemblies. Bioinformatics 27, 2957-2963 (2011), incorporated herein by reference in its entirety) with a minimum sequence length of 70. Merged reads were then mapped using bowtie2 (Langmead, B. & Salzberg, S. L. Fast gapped-read alignment with Bowtie 2. Nature methods 9, 357-359 (2012), incorporated herein by reference in its entirety) with the—very-sensitive setting, and subsequently filtered to restrict to reads with a minimum MAPQ score of 1. The numbers of reads mapping to each synthetic intron in the library were then computed.

Depletion/enrichment of each synthetic intron in the library was estimated similarly as described above for the mini-screen, with modifications to take advantage of the six replicates available for the full screen. The procedure was:
Compute fractional representation for each intron by normalizing the number of reads mapping to that intron to the total number of mapped reads for a given sample.
Compute a standard deviation for the fractional representation of each intron using the sample proportion standard deviation.
Compute the mean of the fractional representation of each intron across all six replicates.
Compute the standard deviation for the mean fractional representation of each intron using error propagation rules for multiplication and division (e.g., ipl.physics.harvard.edu/wp-uploads/2013/03/PS3_Error Propagation_sp13.pdf)
Compute the mean relative fold-change for each intron as its mean fractional representation at the day 8 time point divided by its mean fractional representation at the day 0 time point. Compute standard deviation for this mean relative fold-change using error propagation.

Because of the relative, rather than absolute, nature of fold-changes estimated via sequencing, depletion of one intron necessarily implies that at least one other intron must be enriched (e.g., if one intron has few assigned reads because it has dropped out due to cell death, then another intron must have more assigned reads, simply because a fixed number of cells are collected from each sample, and then a fixed number of reads is sequenced from each sample). Accordingly, it was observed that although longer (˜150 nt) introns exhibited both enrichment and depletion that was concordant with single-construct studies, all very short introns exhibited relative enrichment for both genotypes, including 100 nt control introns which lacked splice sites. Therefore, relative fold-changes for very short (length <115 nt) introns were further normalized by dividing by the mean of fold-changes associated with four 100 nt control introns which lacked splice sites (no 5's, cryptic 3′ss at −11, or canonical 3′ss) in each genotype individually.

Relationships between differences in 3′ splice site strengths and mutation-dependent responses (FIGS. 3D and 7C) were analyzed as follows. All AG dinucleotides that occurred in each intron sequence were identified. The corresponding 23 nt of context (20 nt before the intron-exon junction and 3 nt after) that define each such candidate 3′ss were extracted and used to compute a 3′ss strength with MaxEntScan (Yeo, G. & Burge, C. B. Maximum entropy modeling of short sequence motifs with applications to RNA splicing signals. Journal of computational biology: a journal of computational molecular cell biology 11, 377-394 (2004), incorporated herein by reference in its entirety). After computing these MaxEntScan scores for all candidate 3′ss in an intron, the difference in strength between the two most intron-distal 3′ss was computed as (score for most intron-distal 3′ss—score for next most intron-distal 3′ss). This is equivalent to comparing the difference in splice site strength between the most intron-distal cryptic 3′ splice site with the canonical 3′ splice site, unless the canonical 3′ splice site is ablated by the mutation, in which case two cryptic 3′ splice sites will be compared. This analysis was restricted to introns derived by introducing one or two single-nucleotide mutations to synMTERFD3i1-150 (FIG. 3D) or synMTERFD3i1-100 (FIG. 8C).

Single nucleotide-level analyses (FIGS. 3G and 8F) were performed as follows. Introns derived from deletion scanning, 3′ss conversion, Y>G conversion, and consensus branchpoint insertion:
At each position where a modification was performed, compute the geometric mean and geometric standard deviation over the corresponding construct and its two closest neighbors (three constructs total). Geometric standard deviation is calculated over the fold-changes for the three relevant constructs.
Compute a confidence interval (illustrated by shading on ribbon plot) as the geometric mean scaled by the geometric standard deviation.
   Introns derived by single-nucleotide mutations:
Compute log₂ (mean fold-change) for each construct. For a construct with the mutation X>Y, illustrate this value as the corresponding height of the nucleotide Y in the sequence logo.
   Arc diagrams of combinatorial mutations:
For a given combinatorial mutation X₁>Y₁; X₂>Y₂, compute the expected fold-change based on the corresponding two single-nucleotide mutations X₁>Y₁ and X₂>Y₂ as (fold-change for single-nucleotide mutation construct X₁>Y₁)×(fold-change for single-nucleotide mutation construct X₂>Y₂).
For a given pair of positions (X₁, X₂), compute the geometric mean over all fold-changes for with each associated combinatorial mutation (X₁>Y₁; X₂>Y₂, where X₁ and X₂ are fixed and Y₁ and Y₂ vary over all 4×4 combinations of mutations). Similarly, compute the geometric mean over all expected fold-changes based on the corresponding single-nucleotide mutations (X₁>Y₁ and X₂>Y₂, where X₁ and X₂ are fixed and Y₁ and Y₂ vary over all 4×4 combinations of mutations).
Estimate dinucleotide interaction (synergy) between nucleotides X₁ and X₂ as (geometric mean over observed fold-changes for combinatorial mutations affecting those positions)/(geometric mean over expected fold-changes based on single-nucleotide mutations affecting those positions).

gDNA PCR for Sequencing

gDNA was extracted using the DNeasy Blood and Tissue Kit (Qiagen) following the manufacturer's protocol. Intronic regions of interest were amplified using primers listed in TABLE 3 and analyzed using agarose gel electrophoresis to verify library size after amplifying from gDNA, adding Illumina adapters and adding Illumina barcodes.

SF3B1 Mutation Identification

Samples bearing recurrent SF3B1 mutations (FIGS. 1A and 1B) were identified by searching for RNA-seq reads with single-nucleotide variants corresponding to known, high-frequency mutations in SF3B1 with rnasegmut (see github.com/davidliwei/rnaseqmut).

RNA-Seq Analysis

Splicing events that were particularly responsive to SF3B1 mutations (FIG. 1B) were identified as follows. Transcriptome-wide alternative splicing analysis was performed as previously described (Ilagan, J. O. et al. U2AF1 mutations alter splice site recognition in hematological malignancies. Genome research 25, 14-26 (2015), incorporated herein by reference in its entirety). In brief, a gene and isoform annotation of the GRCh37/hg19 genome assembly was created by merging annotations from Ensembl v71.1 (Flicek, P. et al. Ensembl 2013. Nucleic acids research 41, D48-55 (2013), incorporated herein by reference in its entirety), UCSC knownGene (Meyer, L. R. et al. The UCSC Genome Browser database: extensions and updates 2013. Nucleic acids research 41, D64-9 (2013), incorporated herein by reference in its entirety), and MISO v2.0 (Katz, Y., Wang, E. T., Airoldi, E. M. & Burge, C. B. Analysis and design of RNA sequencing experiments for identifying isoform regulation. Nature methods 7, 1009-1015 (2010), incorporated herein by reference in its entirety) annotations. RNA-seq reads were mapped to this transcriptome annotation with RSEM v1.2.4 (Li, B. & Dewey, C. N. RSEM: accurate transcript quantification from RNA-Seq data with or without a reference genome. BMC bioinformatics 12, 323 (2011), incorporated herein by reference in its entirety), and remaining unaligned reads were mapped to the genome and a database of all possible junctions between annotated 5′ and 3′ splice sites within single genes with TopHat v2.0.8b (Trapnell, C., Pachter, L. & Salzberg, S. L. TopHat: discovering splice junctions with RNA-Seq. Bioinformatics (Oxford, England) 25, 1105-1111 (2009), incorporated herein by reference in its entirety). Isoform expression was computed with MISO v2.0, and candidate SF3B1 mutation-responsive events were identified as previously described (Inoue, D. et al. Spliceosomal disruption of the non-canonical BAF complex in cancer. Nature (2019)). The resulting initial list of SF3B1 mutation-responsive events was then further filtered to those that exhibited the most consistent responses across cancer types by restricting to events with an absolute mean change in isoform ratio >0.1 and standard deviation of isoform ratio <0.17. This loose standard deviation cutoff was chosen to permit some variability in SF3B1 mutation-dependent differential splicing across cohorts, while still eliminating splicing events that exhibited unwanted large variation. Cassette exons, competing 5′ or 3′ splice sites, or annotated retained introns for which no isoform ratio could be computed due to insufficient read counts in >15% of individual samples were eliminated from further consideration. The final set of six events selected for experimental studies (FIG. 1F) were chosen based on manual inspection of RNA-seq read coverage across patient samples (to confirm robust differential splicing and eliminate events whose splicing was very complex, such as those involving multiple linked types of differential splicing).

Data Availability

RNA-seq data from 16 normal human tissues (Illumina Body Map 2.0, illustrated in FIG. 1A) was downloaded from EMBL-EBI ArrayExpress (accession E-MTAB-513). RNA-seq data from published studies was downloaded from CGHub (TCGA cohorts), the Genomic Data Commons (accession BEATAML1.0-COHORT for the Beat AML cohort (Tyner, J. W. et al. Functional genomic landscape of acute myeloid leukaemia. Nature 60, 277-531 (2018), incorporated herein by reference in its entirety)), the Gene Expression Omnibus (accession GSE72790 for chronic lymphocytic leukemia (Darman, R. B. et al. Cancer-Associated SF3B1 Hotspot Mutations Induce Cryptic 3? Splice Site Selection through Use of a Different Branch Point. Cell reports 13, 1033-1045 (2015), incorporated herein by reference in its entirety), GSE49642 for acute myeloid leukemia (Lavallée, V.-P. et al. The transcriptomic landscape and directed chemical interrogation of MLL-rearranged acute myeloid leukemias. Nature genetics (2015) doi:10.1038/ng.3371, incorporated herein by reference in its entirety), GSE63569 and GSE85712 for myelodysplastic syndromes (Obeng, E. A. et al. Physiologic Expression of Sf3b1(K700E) Causes Impaired Erythropoiesis, Aberrant Splicing, and Sensitivity to Therapeutic Spliceosome Modulation. Cancer cell 30, 404-417 (2016); and Dolatshad, H. et al. Disruption of SF3B1 results in deregulated expression and splicing of key genes and pathways in myelodysplastic syndrome hematopoietic stem and progenitor cells. Leukemia: official journal of the Leukemia Society of America, Leukemia Research Fund, UK (2014) doi:10.1038/leu.2014.331, incorporated herein by reference in its entirety)), and dbGaP (myelodysplastic syndromes (Taylor, J. et al. Single-cell genomics reveals the genetic and molecular bases for escape from mutational epistasis in myeloid neoplasms. Blood 136, 1477-1486 (2020), incorporated herein by reference in its entirety)), or obtained directly from the authors (uveal melanoma (Alsafadi, S. et al. Cancer-associated SF3B1 mutations affect alternative splicing by promoting alternative branchpoint usage. Nature communications 7, 10615 (2016), incorporated herein by reference in its entirety). High-throughput sequencing data generated as part of this study was deposited in the Gene Expression Omnibus (GEO accession GSE163217).

Code Availability

Software and algorithms used for analyzing alternative splicing in RNA-seq data, identifying SF3B1-mutant samples, and mapping reads from the screens are published and described with citations in the relevant sections in the Methods section.

Statistics and Reproducibility

All sample sizes are specified in figure legends. Statistical tests are specified in figure legends and in relevant sections in the Methods section. For all box plots, the middle line, hinges, notches, whiskers, and points indicate the median, 25^{th}/75^{th} percentiles, 95% o confidence interval for the median, most extreme data points within 1.5× the interquartile range from the hinge, and outliers.

### Example 1—Tables

TABLE 1
Mini-library composition and results from mini-screen. Table specifying the
sequences of each synthetic intron queried in the mini-screen (FIGS. 2A-21) and
associated fold-changes in WT and SF3B1-mutant K562 cells. Each row corresponds to a
single fold-change measurement for a single synthetic intron. Columns are as follows. id:
intron ID; modification_type: type of modification; modification_location: position(s)
within intron where modifications were applied; length: intron length in nt; genotype:
SF3B1 genotype (WT is SF3B1^{+/+}; K700E is SF3B1^{+/K700E}); fold-change: estimated fold-
change in intron abundance in gDNA at day 6 relative to day 0; sd: standard deviation of
fold-change over replicates; sequence: intron sequence. Note that IDs from the mini-library
do not correspond to IDs from the full library.

| | | | | | | | Sequence |
|---|---|---|---|---|---|---|---|
| | Mod | | | geno- | fold- | | (reference SEQ ID |
| ID | type | Mod location | length | type | change | sd | NO in parentheses) |
| intron_ | mut | GT_to_CC + | 150 | K700E | 0.896076 | 0.006389 | CCGAGTCGCCCCCTT |
| 13121 | | AG_to_CC | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCCC (56) |
| intron_ | del | 15to65 | 100 | K700E | 0.499612 | 0.00631 | GTGAGTCGCCCCCTT |
| 2258 | | | | | | | AGGCCTTGTAGGCC |
| (syn- | | | | | | | ATGGTAAGGCATTT |
| MTERFD3i1- | | | | | | | CTATGTTTTATTTTA |
| 100) | | | | | | | CTTTGTCTTTATCCT |
| | | | | | | | AAAATGCCATTGGC |
| | | | | | | | AAGTTTATTGCAG |
| | | | | | | | (57) |
| intron_ | del | 25to125 | 150 | K700E | 0.28204 | 0.001871 | GTGAGTCGCCCCCTT |
| 26 | | | | | | | CCTCTGCTCCGAGA |
| (syn- | | | | | | | GGGAAATGGGAATT |
| MTERFD3i1- | | | | | | | AGGGTGGTGGCAGA |
| 150) | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCAG (58) |
| intron_ | mut | 148_AtoG | 150 | K700E | 0.761051 | 0.004294 | GTGAGTCGCCCCCTT |
| 2790 | | | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCGG (59) |
| intron_ | comb | 138_AtoC- | 150 | K700E | 0.26371 | 0.002317 | GTGAGTCGCCCCCTT |
| 4095 | mut | 139_GtoC | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCACCTTTAT |
| | | | | | | | TGCAG (60) |
| intron_ | comb | 89_AtoG- | 150 | K700E | 0.27667 | 0.002316 | GTGAGTCGCCCCCTT |
| 8499 | mut | 95_AtoG- | | | | | CCTCTGCTCCGAGA |
| | | 102_AtoG- | | | | | GGGAAATGGGAATT |
| | | 107_AtoG | | | | | AGGGTGGTGGCAGA |
| | | | | | | | GCCCAAAGAGGCCT |
| | | | | | | | TGTAGGCCATGGTA |
| | | | | | | | AGGCGTTTCTGTGTT |
| | | | | | | | TTGTTTTGCTTTGTC |
| | | | | | | | TTTATCCTAAAATGC |
| | | | | | | | CATTGGCAAGTTTAT |
| | | | | | | | TGCAG (61) |
| intron_ | ins | 89_TACTA | 58 | K700E | 0.235605 | 0.002532 | GTGAGTCGCCCCCTT |
| 9600 | | ACA | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCTACTAACAAT |
| | | | | | | | TTCTATGTTTTATTT |
| | | | | | | | TACTTTGTCTTTATC |
| | | | | | | | CTAAAATGCCATTG |
| | | | | | | | GCAAGTTTATTGCA |
| | | | | | | | G (62) |
| intron_ | NA | NA | 250 | K700E | 0.23 | 0.005399 | GTGAGTCGCCCCCTT |
| control | | | | | | | CCTCTGCTCCTGGGC |
| (syn- | | | | | | | GTGTTCCTCACCAGC |
| MTERFD3i1- | | | | | | | GGCGCCGCAGCGGT |
| 250) | | | | | | | CAGGGCCCGCAAAA |
| | | | | | | | CCCCACGCCTCGCC |
| | | | | | | | AGACGCTCAGCTCC |
| | | | | | | | AGGAAGCAAATGCA |
| | | | | | | | GCTGGTGCAGGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCAG (63) |
| intron_ | mut | GT_to_CC + | 150 | WT | 1.225541 | 0.004382 | CCGAGTCGCCCCCTT |
| 13121 | | AG_to_CC | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCCC (64) |
| intron_ | del | 15to65 | 100 | WT | 1.278729 | 0.006912 | GTGAGTCGCCCCCTT |
| 2258 | | | | | | | AGGCCTTGTAGGCC |
| (syn- | | | | | | | ATGGTAAGGCATTT |
| MTERFD3i1- | | | | | | | CTATGTTTTATTTTA |
| 100) | | | | | | | CTTTGTCTTTATCCT |
| | | | | | | | AAAATGCCATTGGC |
| | | | | | | | AAGTTTATTGCAG |
| | | | | | | | (65) |
| intron_ | del | 25to125 | 150 | WT | 1.033777 | 0.002793 | GTGAGTCGCCCCCTT |
| 26 | | | | | | | CCTCTGCTCCGAGA |
| (syn- | | | | | | | GGGAAATGGGAATT |
| MTERFD3i1- | | | | | | | AGGGTGGTGGCAGA |
| 150) | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCAG (66) |
| intron_ | mut | 148_AtoG | 150 | WT | 1.042471 | 0.003423 | GTGAGTCGCCCCCTT |
| 2790 | | | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCGG (67) |
| intron_ | comb | 138_AtoC- | 150 | WT | 0.330503 | 0.001721 | GTGAGTCGCCCCCTT |
| 4095 | mut | 139_GtoC | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCACCTTTAT |
| | | | | | | | TGCAG (68) |
| intron_ | comb | 89_AtoG- | 150 | WT | 1.149932 | 0.003774 | GTGAGTCGCCCCCTT |
| 8499 | mut | 95_AtoG- | | | | | CCTCTGCTCCGAGA |
| | | 102_AtoG- | | | | | GGGAAATGGGAATT |
| | | 107_AtoG | | | | | AGGGTGGTGGCAGA |
| | | | | | | | GCCCAAAGAGGCCT |
| | | | | | | | TGTAGGCCATGGTA |
| | | | | | | | AGGCGTTTCTGTGTT |
| | | | | | | | TTGTTTTGCTTTGTC |
| | | | | | | | TTTATCCTAAAATGC |
| | | | | | | | CATTGGCAAGTTTAT |
| | | | | | | | TGCAG (69) |
| intron_ | ins | 89_TACTA | 158 | WT | 1.112399 | 0.0045 | GTGAGTCGCCCCCTT |
| 9600 | | ACA | | | | | CCTCTGCTCCGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCTACTAACAAT |
| | | | | | | | TTCTATGTTTTATTT |
| | | | | | | | TACTTTGTCTTTATC |
| | | | | | | | CTAAAATGCCATTG |
| | | | | | | | GCAAGTTTATTGCA |
| | | | | | | | G (70) |
| intron | NA | NA | 250 | WT | 0.96 | 0.009833 | GTGAGTCGCCCCCTT |
| control | | | | | | | CCTCTGCTCCTGGGC |
| (syn- | | | | | | | GTGTTCCTCACCAGC |
| MTERFD3i1- | | | | | | | GGCGCCGCAGCGGT |
| 250) | | | | | | | CAGGGCCCGCAAAA |
| | | | | | | | CCCCACGCCTCGCC |
| | | | | | | | AGACGCTCAGCTCC |
| | | | | | | | AGGAAGCAAATGCA |
| | | | | | | | GCTGGTGCAGGAGA |
| | | | | | | | GGGAAATGGGAATT |
| | | | | | | | AGGGTGGTGGCAGA |
| | | | | | | | GCCCAAAGAGGCCT |
| | | | | | | | TGTAGGCCATGGTA |
| | | | | | | | AGGCATTTCTATGTT |
| | | | | | | | TTATTTTACTTTGTC |
| | | | | | | | TTTATCCTAAAATGC |
| | | | | | | | CATTGGCAAGTTTAT |
| | | | | | | | TGCAG (71) |

TABLE 2
Sequence modifications represented in full library. Table specifying numerical
breakdown of full library by parent synthetic intron and modification type(s) used to
create each class of intron variant.

| | Number | | |
|---|---|---|---|
| Modification | of Intron | | |
| ID | Variants | Parent Intron | Description |
| mod1a | 151 | synMTERFD3i1-250 | Deletion scanning: 100 nt. |
| mod1b | 126 | synMTERFD3i1-250 | Deletion scanning: 125 nt |
| mod1c | 101 | synMTERFD3i1-250 | Deletion scanning: 150 nt |
| mod1d | 146 | synMTERFD3i1-150 | Deletion scanning: 5 nt |
| mod1e | 141 | synMTERFD3i1-150 | Deletion scanning: 10 nt |
| mod1f | 136 | synMTERFD3i1-150 | Deletion scanning: 15 nt |
| mod1g | 131 | synMTERFD3i1-150 | Deletion scanning: 20 nt |
| mod1h | 126 | synMTERFD3i1-150 | Deletion scanning: 25 nt |
| mod1i | 121 | synMTERFD3i1-150 | Deletion scanning: 30 nt |
| mod1j | 116 | synMTERFD3i1-150 | Deletion scanning: 35 nt |
| mod1k | 111 | synMTERFD3i1-150 | Deletion scanning: 40 nt |
| mod1l | 106 | synMTERFD3i1-150 | Deletion scanning: 45 nt |
| mod1m | 101 | synMTERFD3i1-150 | Deletion scanning: 50 nt |
| mod1n | 96 | synMTERFD3i1-100 | Deletion scanning: 5 nt |
| mod1o | 91 | synMTERFD3i1-100 | Deletion scanning: 10 nt |
| mod1p | 86 | synMTERFD3i1-100 | Deletion scanning: 15 nt |
| mod1q | 81 | synMTERFD3i1-100 | Deletion scanning: 20 nt |
| mod1r | 76 | synMTERFD3i1-100 | Deletion scanning: 25 nt |
| mod2a | 450 | synMTERFD3i1-150 | Single nucleotide mutagenesis. |
| | | | Saturation mutagenesis at every |
| | | | position |
| mod2b | 300 | synMTERFD3i1-100 | Single nucleotide mutagenesis. |
| | | | Saturation mutagenesis at every |
| | | | position |
| mod3 | 1710 | synMTERFD3i1-150 | Combinatorial single-nucleotide |
| | | | mutagenesis. Saturation |
| | | | mutagenesis of every pair of |
| | | | nucleotides within positions −22 to |
| | | | −1 relative to the 3'ss. |
| mod5a | 36 | synMTERFD3i1-150 | Combinatorial branchpoint |
| | | | ablation. Mutate all known |
| | | | branchpoints from A to G then |
| | | | identify the 9 closest adenines to |
| | | | the canonical 3'ss and mutate every |
| | | | combination of 2 adenines to |
| | | | guanines (A > G). |
| mod5b | 84 | synMTERFD3i1-150 | Combinatorial branchpoint |
| | | | ablation. Mutate all known |
| | | | branchpoints from A to G then |
| | | | identify the 9 closest adenines to |
| | | | the canonical 3'ss and mutate every |
| | | | combination of 3 adenines (A > G). |
| mod5c | 126 | synMTERFD3i1-150 | Combinatorial branchpoint |
| | | | ablation. Mutate all known |
| | | | branchpoints from A to G then |
| | | | identify the 9 closest adenines to |
| | | | the canonical 3'ss and mutate every |
| | | | combination of 4 adenines (A > G). |
| mod6 | 10 | synMTERFD3i1-150 | Combinatorial branchpoint |
| | | | strengthening. Mutate all possible |
| | | | combinations of known |
| | | | branchpoint contexts to the |
| | | | branchpoint consensus (tactaAca). |
| | | | 4 known branchpoints were |
| | | | identified through sequencing data. |
| mod7a | 75 | synMTERFD3i1-150 | Sliding branchpoint conversion. |
| | | | Convert each consecutive 8 nt to |
| | | | the branchpoint consensus |
| | | | (tactaAca) within positions [−75, |
| | | | −1] relative to the canonical 3'ss |
| mod7b | 75 | synMTERFD3i1-100 | Sliding branchpoint conversion. |
| | | | Convert each consecutive 8 nt to |
| | | | the branchpoint consensus |
| | | | (tactaAca) within positions [−75, |
| | | | −1] relative to the canonical 3'ss |
| mod7c | 75 | synMTERFD3i1-150 | Branchpoint ablation and sliding |
| | | | branchpoint conversion. Mutate all |
| | | | known branchpoints (A > G), then |
| | | | convert each consecutive 8 nt to |
| | | | the branchpoint consensus |
| | | | (tactaAca) within positions [−75, |
| | | | −1] relative to the canonical 3'ss |
| mod7d | 75 | synMTERFD3i1-100 | Branchpoint ablation and sliding |
| | | | branchpoint conversion. Mutate all |
| | | | known branchpoints (A > G), then |
| | | | convert each consecutive 8 nt to |
| | | | the branchpoint consensus |
| | | | (tactaAca) within positions [−75, |
| | | | −1] relative to the canonical 3'ss |
| mod8a | 75 | synMTERFD3i1-150 | Sliding branchpoint insertion. |
| | | | Insert branchpoint consensus |
| | | | (tactaAca) at each position within |
| | | | positions [−75, −1] relative to the |
| | | | canonical 3'ss |
| mod8b | 150 | synMTERFD3i1-100 | Sliding branchpoint insertion. |
| | | | Insert one or two branchpoint |
| | | | consensus sequences (tactaAca) at |
| | | | each position within positions [-75, |
| | | | -1] relative to the canonical 3'ss. |
| | | | Note, because the 100 bp intron is |
| | | | shorter, the extra sequence space |
| | | | was used to test having tandem |
| | | | optimal branchpoint sequences |
| mod8c | 75 | synMTERFD3i1-150 | Branchpoint ablation and sliding |
| | | | branchpoint insertion. Mutate all |
| | | | known branchpoints (A > G), then |
| | | | insert branchpoint consensus |
| | | | (tactaAca) at each position within |
| | | | positions [−75, −1] relative to the |
| | | | canonical 3'ss |
| mod8d | 75 | synMTERFD3i1-100 | Branchpoint ablation and sliding |
| | | | branchpoint insertion. Mutate all |
| | | | known branchpoints (A > G), then |
| | | | insert branchpoint consensus |
| | | | (tactaAca) at each position within |
| | | | positions [−75, −1] relative to the |
| | | | canonical 3's |
| mod9 | 74 | synMTERFD3i1-150 | 3'ss conversion. Convert each |
| | | | consecutive 4 nt to the 3'ss CAGG |
| | | | within positions [−75, −1] relative |
| | | | to the canonical 3'ss |
| mod10 | 20 | synMTERFD3i1-150 | Polypyrimidine tract conversion: |
| | | | convert each consecutive 6 nt to |
| | | | the polypyrimidine tract TTTTTT |
| | | | within positions [−75, −1] relative |
| | | | to the canonical 3'ss |
| modlla | 76 | synMTERFD3i1-150 | Pyrimidine ablation: convert each |
| | | | pyrimidine within each |
| | | | consecutive 6 nt to G within |
| | | | positions [−75, −1] relative to the |
| | | | canonical 3'ss |
| mod11b | 75 | synMTERFD3i1-100 | Pyrimidine ablation: convert each |
| | | | pyrimidine within each |
| | | | consecutive 6 nt to G within |
| | | | positions [−75, −1] relative to the |
| | | | canonical 3'ss |
| mod12a | 75 | synMTERFD3i1-150 | Polypyrimidine tract and 3'ss |
| | | | conversion: convert each |
| | | | consecutive 20 nt to the |
| | | | polypyrimidine tract and 3'ss |
| | | | TTTTTTTTTTTTTTTTTCAG |
| | | | (SEQ ID NO: 72) within positions |
| | | | [−75, −1] relative to the canonical |
| | | | 3'ss |
| mod12b | 74 | synMTERFD3i1-100 | Polypyrimidine tract and 3'ss |
| | | | conversion: convert each |
| | | | consecutive 20 nt to the |
| | | | polypyrimidine tract and 3'ss |
| | | | TTTTTTTTTTTTTTTTTCAG |
| | | | (SEQ ID NO: 72) within positions |
| | | | [−75, −1] relative to the canonical |
| | | | 3'ss |
| mod13a | 43 | synMTERFD3i1-150 | Removal of internal adenines and |
| | | no As | sliding branchpoint insertion: |
| | | | Remove all adenines from the |
| | | | intron except for the A in the |
| | | | canonical 3'ss then then insert |
| | | | branchpoint consensus (tactaAca) |
| | | | at each position 75 bp beyond the |
| | | | 5'ss. Note after removal of all |
| | | | internal As the intron is 118 bp |
| | | | long |
| mod13b | 56 | synMTERFD3i1-100 | Removal of internal adenines and |
| | | | sliding branchpoint insertion: |
| | | | Remove all adenines from the |
| | | | intron except for the A in the |
| | | | canonical 3'ss then then insert |
| | | | branchpoint consensus (tactaAca) |
| | | | at each position 25 bp beyond the |
| | | | 5'ss. Note after removal of all |
| | | | internal As the parent intron is |
| | | | 81 bp long |
| mod14a | 54 | synMTERFD3i1-150 | Combinatorial single-nucleotide |
| | | | mutagenesis of 5'ss: saturation |
| | | | mutagenesis of every pair of |
| | | | nucleotides within positions [+1, |
| | | | +6] relative to the 5'ss |
| mod14b | 54 | synMTERFD3i1-100 | Combinatorial single-nucleotide |
| | | | mutagenesis of 5'ss: saturation |
| | | | mutagenesis of every pair of |
| | | | nucleotides within positions [+1, |
| | | | +6] relative to the 5'ss |
| mod15 | 36 | synMTERFD3i1-100 | Sliding endogenous intronic |
| | | | insertion. Split the endogenous |
| | | | intron into 35 50 bp sequences and |
| | | | 1 last 38 bp sequence then insert |
| | | | each at position +15 relative to the |
| | | | 5'ss |
| mod16 | 2000 | synMTERFD3i1-100 | Sliding insertion of polypyrimidine |
| | | | tract, splice site and branchpoint: |
| | | | Cut down the 100 bp synthetic |
| | | | intron to 74 bp by taking |
| | | | nucleotides lto 30 from the 5'ss |
| | | | and −44 to −1 from the 3'ss. Insert |
| | | | TTTTTTTTTTTTTTTTTCAG |
| | | | (SEQ ID NO: 72) in a sliding |
| | | | window in the −30 to the −1 |
| | | | position relative to the 3'ss. Then, |
| | | | insert 1-5 consensus BP sequences |
| | | | in position 25-50 relative to the |
| | | | 5'ss |
| mod18a | 48 | synMTERFD3i1-150 | Sliding intronic splicing enhancer |
| | | | insertion: insert 1 to 4 consecutive |
| | | | repeats of intronic splicing |
| | | | enhancers (groups E and F from |
| | | | nature.com/articles/nsmb.2377) |
| | | | immediately prior to the cryptic |
| | | | 3'ss relative to the canonical 3'ss |
| | | | (Position −12 immediately after the |
| | | | 3'ss) |
| mod18b | 48 | synMTERFD3i1-150 | Sliding intronic splicing enhancer |
| | | | insertion: insert 1 to 4 consecutive |
| | | | repeats of intronic splicing |
| | | | enhancers (groups E and F from |
| | | | Wang et al., Nature Structural & |
| | | | Molecular Biology volume 19, |
| | | | pages1044-1052(2012), |
| | | | incorporated herein by reference in |
| | | | its entirety) at position −33 |
| | | | (proceeding 32 nucleotides at the |
| | | | 3' end of the intron) |
| mod19a | 12 | synMTERFD3i1-150 | Starting with nucleotide 0-10 from |
| | | | the 5'ss mutate every |
| | | | 2nd , 3rd , 4th ,5th or 10th nucleotide |
| | | | to the corresponding pair. Most of |
| | | | these variants are already covered |
| | | | in other single and double |
| | | | mutagenesis screens or result in |
| | | | duplicates |
| mod19b | 600 | synMTERFD3i1-150 | Random intronic shuffle: randomly |
| | | | shuffle all nucleotides within |
| | | | positions [+10, −30] relative to the |
| | | | 5'ss and canonical 3'ss |
| mod19c | 541 | synMTERFD3i1-100 | Random intronic shuffle: randomly |
| | | | shuffle all nucleotides within |
| | | | positions [+10, −30] relative to the |
| | | | 5'ss and canonical 3'ss |
| modneg | 200 | synMTERFD3i1-150 or | For each intron length the 5'ss, |
| | | synMTERFD3i1-100 | canonical 3'ss and alternative (AG) |
| | | | 3'ss to AA, TT, CC to GG (25 |
| | | | copies of each) were ablated. A |
| | | | random internal point mutation |
| | | | was then created within each of the |
| | | | 25 copies to be able to distinguish |
| | | | them if needed in the downstream |
| | | | analysis. For simplicity in mapping |
| | | | for the analysis in the paper each |
| | | | variant was grouped by the splice |
| | | | site mutation providing 4 controls |
| | | | for each intron length |

TABLE 3
PCR primers and other oligos. Table specifying sequences of all
oligonucleotides used in this study for PCR, cloning, etc.

| | | Binding | | Sequence | |
|---|---|---|---|---|---|
| | | Gene | | (Sequence identifiers | |
| Primer ID | Reporter | Region | Strand | in parentheses) | Note |
| RKB4175 | Bidirectional | mEmerald | F | ACGACGGCAACTACAAG | reporter |
| | Fluorescent | | | ACC (73) | |
| RKB4176 | Bidirectional | mEmerald | R | TGTAGTTGTACTCCAGCT | reporter |
| | Fluorescent | | | TGTGC (74) | |
| RKB4019 | Endogenous | MAP3K7 | F | TGTCTTGTGATGGAATAT | exon 4 |
| | | | | GCTG (75) | |
| RKB4020 | Endogenous | MAP3K7 | R | TCCCTGTGAATTAGCGCT | exon 5 |
| | | | | TT (76) | |
| RKB4185 | Endogenous | MTERFD3 | F | ACTCCCTGTGCCTTGCTT | exon 1 |
| | | | | G (77) | |
| RKB4186 | Endogenous | MTERFD3 | F | CATTTTGGGCATGGAATC | intron 1 |
| | | | | TG (78) | |
| RKB4187 | Endogenous | MTERFD3 | R | ATGGACTCATTCTATCTT | exon 2/3 |
| | | | | ACAGTCTCTCC (79) | |
| RKB4015 | Endogenous | ORAI2 | F | AGTCCGAGCGGGAGCCG | exon 1 |
| | | | | AGC (80) | |
| RKB4016 | Endogenous | ORAI2 | R | AGCAGCACCGCATCCAAG | exon 2 |
| | | | | CAC (81) | |
| RKB4173 | Endogenous | TMEM14C | F | GACACCTCGCAGTCATTC | exon 1 |
| | | | | CT (82) | |
| RKB4174 | Endogenous | TMEM14C | R | GTAGCCAAAGCCAAACC | exon 3 |
| | | | | AATG (83) | |
| RKB1947 | Endogenous | MYO15B | F | GTGGCATCAGCCTATGAC | exon 4 |
| | | | | CT (84) | |
| RKB1948 | Endogenous | MYO15B | R | TCCAGGCTGCTTAGGAAC | exon 5 |
| | | | | TG (85) | |
| RKB1949 | Endogenous | SYTL1 | F | AAGCTACCTCCTCCCGGA | exon 10 |
| | | | | TA (86) | |
| RKB1950 | Endogenous | SYTL1 | R | CTTCAACTTCGCCCAGAA | exon 11 |
| | | | | AG (87) | |
| RKB2453 | Bidirectional | GAPDH | R | GTCAGATCGCTCAGGTGT | mCardinal |
| | Fluorescent | UTR | | CGTGAGCCTC (88) | and GAPDH |
| | | | | | UTR |
| RKB2454 | Bidirectional | SV40 | F | CAGAGGTTGATTGTCGAG | mCardinal |
| | Fluorescent | poly A tail | | GCGCTTGATATCGAATTC | and GAPDH |
| | | | | CCACTAAG (89) | UTR |
| RKB2451 | Bidirectional | pBI CMV | R | CGACACCTGAGCGATCTG | pBi CMV |
| | Fluorescent | | | ACGGTTCACTAAAC (90) | |
| RKB2452 | Bidirectional | pBI CMV. | F | CCAGCCCAAGGTCTTGAG | pBi CMV |
| | Fluorescent | And | | GCAGCGGATCTGACGGTT | |
| | | GAPDH | | CAC (91) | |
| | | UTR | | | |
| RKB2455 | Bidirectional | GAPDH | R | GCCTCAAGACCTTGGGCT | Amplifies |
| | Fluorescent | UTR | | G (92) | Backbone |
| RKB2456 | Bidirectional | Backbone | F | GCCTCGACAATCAACCTC | Amplifies |
| | Fluorescent | | | TG (93) | Backbone |
| RKB2511 | Bidirectional | mEmerald | R | TGAAGTTCGAGGGCGACA | linearization |
| | Fluorescent | | | C (94) | of |
| | | | | | mEmerald |
| | | | | | for |
| | | | | | synMAP3K7i4- |
| | | | | | 250 |
| RKB2512 | Bidirectional | mEmerald | F | CCTCGGCGCGGGTCTTGT | linearization |
| | Fluorescent | | | (95) | of |
| | | | | | mEmerald |
| | | | | | for |
| | | | | | synMAP3K7i4- |
| | | | | | 250 |
| RKB2515 | Bidirectional | mEmerald | R | TGAACCGCATCGAGCTGA | linearization |
| | Fluorescent | | | AG (96) | of |
| | | | | | mEmerald |
| | | | | | for |
| | | | | | synSYTL1i10- |
| | | | | | 250 |
| RKB2516 | Bidirectional | mEmerald | F | CCAGGGTGTCGCCCTCGA | linearization |
| | Fluorescent | | | (97) | of |
| | | | | | mEmerald |
| | | | | | for |
| | | | | | synSYTL1i10- |
| | | | | | 250 |
| RKB2521 | Bidirectional | mEmerald | R | GTGAAGTTCGAGGGCGAC | linearization |
| | Fluorescent | | | (98) | of |
| | | | | | mEmerald |
| | | | | | for |
| | | | | | synTEMEM14Ci1- |
| | | | | | 250 |
| RKB2522 | Bidirectional | mEmerald | F | CTCGGCGCGGGTCTTGTA | linearization |
| | Fluorescent | | | (99) | of |
| | | | | | mEmerald |
| | | | | | for |
| | | | | | synTEMEM |
| | | | | | 14Ci1-250 |
| RKB2523 | Bidirectional | mEmerald | R | GGGCACAAGCTGGAGTA | linearization |
| | Fluorescent | | | C (100) | of |
| | | | | | mEmerald |
| | | | | | for |
| | | | | | synORAI2i1- |
| | | | | | 250 |
| RKB2524 | Bidirectional | mEmerald | F | CAGGATGTTGCCGTCCTC | linearization |
| | Fluorescent | | | (101) | of |
| | | | | | mEmerald |
| | | | | | for |
| | | | | | synORAI2i1- |
| | | | | | 250 |
| RKB2525 | Bidirectional | mEmerald | R | CTGAAGGGCATCGACTTC | linearization |
| | Fluorescent | | | (102) | of |
| | | | | | mEmerald |
| | | | | | for |
| | | | | | synMYO15Bi4- |
| | | | | | 250 |
| RKB2526 | Bidirectional | mEmerald | F | CTCGATGCGGTTCACCAG | linearization |
| | Fluorescent | | | (103) | of |
| | | | | | mEmerald |
| | | | | | for |
| | | | | | synMYO15Bi4- |
| | | | | | 250 |
| RKB2527 | Bidirectional | mEmerald | R | GGCGACACCCTGGTGAAC | linearization |
| | | | | | of |
| | Fluorescent | | | (104) | mEmerald |
| | | | | | for |
| | | | | | synMTERFD3i1- |
| | | | | | 250 |
| RKB2528 | Bidirectional | mEmerald | F | CTCGAACTTCACCTCGGC | linearization |
| | Fluorescent | | | (105) | of |
| | | | | | mEmerald |
| | | | | | for |
| | | | | | synMTERFD3i1- |
| | | | | | 250 |
| RKB2541 | Bidirectional | mEmerald | F | CATGCCGAGAGTGATC | sequencing |
| | Fluorescent | | | (106) | primer to |
| | | | | | check for |
| | | | | | intron |
| | | | | | insertion |
| RKB2661 | Bidirectional | pBiCMV | R | TACACGCCACCTCGACAT | mEmerald, |
| | Fluorescent | | | AC (107) | GAPDH |
| | | | | | UTR, |
| | | | | | bidirectional |
| | | | | | promoter |
| | | | | | (pair with F |
| | | | | | primers for |
| | | | | | intron |
| | | | | | specific |
| | | | | | mEmerald |
| | | | | | primers |
| | | | | | above) |
| RKB2662 | Bidirectional | pBiCMV | F | TATGTCGAGGTGGCGTGT | pBiCMV, |
| | Fluorescent | | | AC (108) | GAPDH |
| | | | | | UTR, |
| | | | | | mCardinal, |
| | | | | | WPRE |
| RKB2631 | Bidirectional | WPRE | R | GAGATCCGACTCGTCTGA | pBiCMV, |
| | Fluorescent | | | GG (109) | GAPDH |
| | | | | | UTR, |
| | | | | | mCardinal, |
| | | | | | WPRE |
| RKB2632 | Bidirectional | WPRE | F | CCTCAGACGAGTCGGATC | Backbone |
| | Fluorescent | | | TC (110) | amplification |
| | | | | | (pair with |
| | | | | | a R primer |
| | | | | | for from the |
| | | | | | intron |
| | | | | | specific |
| | | | | | mEmerald |
| | | | | | primers |
| | | | | | above) |
| RKB2491 | Bidirectional | gBlock | gBlock | GATGCCCTTCAGCTCGAT | mEmerald |
| | Fluorescent | | | GCGGTTCACCAGGGTGTC | overhangs |
| | | | | GCCCTCGAACTTCACTGC | and |
| | | | | GAAAGAAAGGCACAAAC | MAP3K7 |
| | | | | TAAAAAGAACTTTATTGC | |
| | | | | ATATAACAAAAGACACA | |
| | | | | ATGTGAATGCAAGCACTA | |
| | | | | TCAGAACACATGACTGGG | |
| | | | | TATTTTTCTACATATACAT | |
| | | | | ATGCTCCATCAGTCCTGC | |
| | | | | TTTTAATTTTTTACTTTCT | |
| | | | | TTGACGGCAAATTGTGAA | |
| | | | | GAATGTTAAACAACTTAG | |
| | | | | GGTTTATATTAAAATTTTT | |
| | | | | CAAATTATTTTAATCCAC | |
| | | | | TAGATAAAGACAGGTCTA | |
| | | | | ATGACACTCACCCTCGGC | |
| | | | | GCGGGTCTTGTAGTTGCC | |
| | | | | GTCGTCCTTGAAGAAGAT | |
| | | | | GGTGCGC (111) | |
| RKB2492 | Bidirectional | gBlock | gBlock | ATGCCCTTCAGCTCGATG | mEmerald |
| | Fluorescent | | | CGGTTCACCAGGGTGTCG | overhangs |
| | | | | CCCTCGAACTTCACCTGC | and |
| | | | | AGAAAACAAGCTGGATC | TMEM14C |
| | | | | AGAGGTAGTTAAAAGCA | |
| | | | | CAGCTCTCCAGCAGTCAG | |
| | | | | AAAGAAGTGTGTCCAACC | |
| | | | | TGCAAGCTACGCAACCTA | |
| | | | | AGTCTCAGTATTCTTCAC | |
| | | | | TGTACCATGAGGATAACA | |
| | | | | CTGGTACCGATTTCCTGG | |
| | | | | GGCTCTGAATCGGGGGGA | |
| | | | | CGAAAAGCAGACGGCCG | |
| | | | | TGAAACTCCTAAATAATC | |
| | | | | CCTAAAAGTTAAAAGTGG | |
| | | | | CAGAAAATAAGAATAAT | |
| | | | | CCCCAAATACTCGCACCT | |
| | | | | CGGCGCGGGTCTTGTAGT | |
| | | | | TGCCGTCGTCCTTGAAGA | |
| | | | | AGATGGTGCGCT (112) | |
| RKB2493 | Bidirectional | gBlock | gBlock | GCGGTGATATAGACCTTG | mEmerald |
| | Fluorescent | | | TGGCTGTTGTAGTTGTAC | overhangs |
| | | | | TCCAGCTTGTGCCCCTTG | and ORAI2 |
| | | | | GGGAGAATTCAGAGAAA | |
| | | | | CGCTGCAATCCGTAGGTT | |
| | | | | AGCTCCACATAATAGAAA | |
| | | | | ATGCTGTAATTTGGAAAC | |
| | | | | TGCATTTTTCCTCTCCCCC | |
| | | | | CACCCCCATAGTTAACAG | |
| | | | | AATTGAGCATGCAGGTCT | |
| | | | | GATGACCGCAGTATACAG | |
| | | | | AGAGTCGTCCCCTTCCCC | |
| | | | | GCCGAGCGTCCCCGGTCC | |
| | | | | AATTCCGGCCCCCCCAGC | |
| | | | | CGCAGGGACCCCGCTCCC | |
| | | | | CGCGCCAGGCCCCCGCGC | |
| | | | | CGCCACACTCACCAGGAT | |
| | | | | GTTGCCGTCCTCCTTGAA | |
| | | | | GTCGATGCCCTTCAGCTC | |
| | | | | GATGCGGT (113) | |
| RKB2494 | Bidirectional | gBlock | gBlock | GATGTTGCCGTCCTCCTT | mEmerald |
| | Fluorescent | | | GAAGTCGATGCCCTTCAG | overhangs |
| | | | | CTCGATGCGGTTCACTGA | and SYTL1 |
| | | | | GGACAGGAGGTGAGCTTT | |
| | | | | AGTGGAGCACTGCTGGTG | |
| | | | | GCCCGCGTAGATGCTGCG | |
| | | | | CGAGTTGGCTGAGGCAGG | |
| | | | | TCCTACCGCGTTCCTACC | |
| | | | | ATGATGGAATTCTGGGGA | |
| | | | | GCCCCTAAGGGCATTAAT | |
| | | | | GGGGACCTGCACTATCAC | |
| | | | | CACTCCGGGCAATGACGT | |
| | | | | TACTGGCTGCCCTTGCCA | |
| | | | | CCTAGCCCCGCAGGAAGG | |
| | | | | GGGCGTCCAGTTCATTAA | |
| | | | | CGGGGAACCGCATCGTGG | |
| | | | | TCACAGCCTCACCCAGGG | |
| | | | | TGTCGCCCTCGAACTTCA | |
| | | | | CCTCGGCGCGGGTCTTGT | |
| | | | | AGTTGCCG (114) | |
| RKB2495 | Bidirectional | gBlock | gBlock | TCCTTGAAGTCGATGCCC | mEmerald |
| | Fluorescent | | | TTCAGCTCGATGCGGTTC | overhangs |
| | | | | ACCAGGGTGTCGCCCTGC | and |
| | | | | AATAAACTTGCCAATGGC | MTERFD3 |
| | | | | ATTTTAGGATAAAGACAA | |
| | | | | AGTAAAATAAAACATAG | |
| | | | | AAATGCCTTACCATGGCC | |
| | | | | TACAAGGCCTCTTTGGGC | |
| | | | | TCTGCCACCACCCTAATT | |
| | | | | CCCATTTCCCTCTCCTGCA | |
| | | | | CCAGCTGCATTTGCTTCC | |
| | | | | TGGAGCTGAGCGTCTGGC | |
| | | | | GAGGCGTGGGGTTTTGCG | |
| | | | | GGCCCTGACCGCTGCGGC | |
| | | | | GCCGCTGGTGAGGAACAC | |
| | | | | GCCCAGGAGCAGAGGAA | |
| | | | | GGGGGCGACTCACCTCGA | |
| | | | | ACTTCACCTCGGCGCGGG | |
| | | | | TCTTGTAGTTGCCGTCGT | |
| | | | | CCTTGAAGA (115) | |
| RKB2496 | Bidirectional | gBlock | gBlock | AGCTTGTGCCCCAGGATG | mEmerald |
| | Fluorescent | | | TTGCCGTCCTCCTTGAAG | overhangs |
| | | | | TCGATGCCCTTCAGCTAA | and |
| | | | | GGATGTGTTGGTTGGTAG | MYO15B |
| | | | | GGTCTCCAGGGAGGGGC | |
| | | | | ACATAGGACCACCCCAGT | |
| | | | | GAACCCAGGAAGTGGGA | |
| | | | | TGTGCCACCCAGAAGTGA | |
| | | | | CAGCTAGCCTGGAGAGAC | |
| | | | | CCCCATAAATGTGGAAAG | |
| | | | | ATAGTCCCAGGGCATGGA | |
| | | | | GAGAGTGGAGCATACCA | |
| | | | | GTGGCTTCCAGTGAAGAC | |
| | | | | CTCAGGCCCCGCCCCATC | |
| | | | | AACCCTCAGTGTGGCCCA | |
| | | | | TGGGTTCAGCCACCCAGG | |
| | | | | AAGGCCACCACTCACCTC | |
| | | | | GATGCGGTTCACCAGGGT | |
| | | | | GTCGCCCTCGAACTTCAC | |
| | | | | CTCGGCGCGGG (116) | |
| RKB3405 | hPGK_HSV- | HSV-TK | R | TGCTACCCGGCCGC (117) | linearization |
| | TK_P2A_ | | | | of HSV-TK |
| | mCherry | | | | for |
| | | | | | synMAP3K7i4- |
| | | | | | 250 |
| RKB3406 | hPGK_HSV- | HSV-TK | F | CAGGAGGGCGGCG (118) | linearization |
| | TK_P2A_ | | | | of HSV-TK |
| | mCherry | | | | for |
| | | | | | synMAP3K7i4- |
| | | | | | 250 |
| RKB3409 | hPGK_HSV- | syn | R | TATCGCGCGGCCGGGTAG | amplification |
| | TK_P2A_ | MAP3K7i4- | | CACTGCGAAAGAAAGGC | of |
| | hmCerry | 250/ | | ACAAAC (119) | synMAP3K7i4- |
| | | HSV-TK | | | 250 for |
| | | | | | HSV-TK |
| RKB3410 | hPGK_HSV- | syn- | F | ATCCCATCGCCGCCCTCC | amplification |
| | TK_P2A_ | MAP3K7i4- | | TGGTGAGTGTCATTAGAC | of |
| | hmCerry | 250/ | | CTGTC (120) | synMAP3K7i4- |
| | | HSV-TK | | | 250 for |
| | | | | | HSV-TK |
| RKB3413 | hPGK_HSV- | syn- | R | CGCCGCGTCCCTGCAATA | amplification |
| | TK_P2A_ | MTERFD3i1- | | AACTTGCCAATGGC (121) | of |
| | mCherry | 250/ | | | synMTERFD3i1- |
| | | HSV-TK | | | 250 |
| | | | | | for HSV-TK |
| RKB3414 | hPGK_HSV- | syn- | F | TATCGGCCGGGTGAGTCG | amplification |
| | TK_P2A_ | MTERFD3i1- | | CCCCCTTCCT (122) | of |
| | mCherry | 250/ | | | synMTERFD3i1- |
| | | HSV-TK | | | 250 |
| | | | | | for HSV-TK |
| RKB3411 | hPGK_HSV- | syn- | R | GGCGACTCACCCGGCCGA | linearization |
| | TK_P2A_ | MTERFD3i1- | | TATCTCACCC (123) | of HSV-TK |
| | mCherry | 250/ | | | for |
| | | HSV-TK | | | synMTERFD3i1- |
| | | | | | 250 |
| RKB3412 | hPGK_HSV- | syn- | F | TTTATTGCAGGGACGCGG | linearization |
| | TK_P2A_ | MTERFD3i1- | | CGGTGGTAAT (124) | of HSV-TK |
| | mCherry | 250/ | | | for |
| | | HSV-TK | | | synMTERFD3i1- |
| | | | | | 250 |
| RKB3450 | hPGK_HSV- | HSV-TK | R | AAGTTGGTGGCTCCGCTT | HSV-TK |
| | TK_P2A_ | | | CCGTTAGCCTCCCCCATC | with P2A |
| | mCherry | | | TC (125) | overhang |
| RKB3451 | hPGK_HSV- | HSV-TK | F | CCCCAGGGGGATCCGCCA | HSV-TK |
| | TK_P2A_ | | | CCATGGCTTCGTACCCCT | with hPGK |
| | mCherry | | | G (126) | overhang |
| RKB3446 | hPGK_HSV- | hPGK | R | GGTGGCGGATCCCCC | linearizing |
| | TK_P2A_ | | | (127) | backbone |
| | mCherry | | | | from |
| | | | | | pRKB386 |
| RKB3447 | hPGK_HSV- | P2A | F | GGAAGCGGAGCCACCAA | linearizing |
| | TK_P2A_ | | | C (128) | backbone |
| | mCherry | | | | from |
| | | | | | pRKB386 |
| RKB3668 | hPGK-Puro- | PuroR | R | AAGTTGGTGGCTCCGCTT | Puro with |
| | P2A- | | | CCGGCACCGGGCTTGCG | P2A |
| | HSV_TK | | | (129) | overhang |
| | | | | | from pLenti- |
| | | | | | GFP |
| RKB3669 | hPGK-Puro- | hPGK | F | AAGGTACCGAGCTCGAAT | hPGK with |
| | P2A- | | | TCGGGTAGGGGAGGCGC | backbone |
| | HSV_TK | | | (130) | overhang |
| | | | | | from |
| | | | | | pLentiGFP |
| RKB3666 | hPGK-Puro- | | F | GAATTCGAGCTCGGTACC | backbone |
| | P2A- | | | (131) | from |
| | HSV_TK | | | | hPGK_HSV- |
| | | | | | TK_P2A_ |
| | | | | | mCherry |
| RKB3667 | hPGK-Puro- | | R | GGCTAGTCTCGTGATCG | backbone |
| | P2A- | | | (132) | from |
| | HSV_TK | | | | hPGK_HSV- |
| | | | | | TK_P2A_ |
| | | | | | mCherry |
| NA | hPGK-Puro- | | gBlock | TATCGATCACGAGACTAG | P2A-HSV- |
| | P2A- | | | CCGCCCAAAGGGAGATCC | TK-WPRE |
| | HSV_TK | | | GACTCGTCTGAGGGCGAA | sequence |
| | | | | GGCGAAGACGCGGAAGA | ordered |
| | | | | GGCCGCAGAGCCGGCAG | from |
| | | | | CAGGCCGCGGGAAGGAA | GENEWIZ |
| | | | | GGTCCGCTGGATTGAGGG | |
| | | | | CCGAAGGGACGTAGCAG | |
| | | | | AAGGACGTCCCGCGCAG | |
| | | | | AATCCAGGTGGCAACACA | |
| | | | | GGCGAGCAGCCATGGAA | |
| | | | | AGGACGTCAGCTTCCCCG | |
| | | | | ACAACACCACGGAATTGT | |
| | | | | CAGTGCCCAACAGCCGAG | |
| | | | | CCCCTGTCCAGCAGCGGG | |
| | | | | CAAGGCAGGCGGCGATG | |
| | | | | AGTTCCGCCGTGGCAATA | |
| | | | | GGGAGGGGGAAAGCGAA | |
| | | | | AGTCCCGGAAAGGAGCT | |
| | | | | GACAGGTGGTGGCAATGC | |
| | | | | CCCAACCAGTGGGGGTTG | |
| | | | | CGTCAGCAAACACAGTGC | |
| | | | | ACACCACGCCACGTTGCC | |
| | | | | TGACAACGGGCCACAACT | |
| | | | | CCTCATAAAGAGACAGCA | |
| | | | | ACCAGGATTTATACAAGG | |
| | | | | AGGAGAAAATGAAAGCC | |
| | | | | ATACGGGAAGCAATAGC | |
| | | | | ATGATACAAAGGCATTAA | |
| | | | | AGCAGCGTATCCACATAG | |
| | | | | CGTAAAAGGAGCAACAT | |
| | | | | AGTTAAGAATACCAGTCA | |
| | | | | ATCTTTCACAAATTTTGT | |
| | | | | AATCCAGAGGTTGATCAG | |
| | | | | TTAGCCTCCCCCATCTCC | |
| | | | | CGGGCAAACGTGCGCGCC | |
| | | | | AGGTCGCAGATCGTCGGT | |
| | | | | ATGGAGCCGGGGGTGGT | |
| | | | | GACGTGGGTCTGGACCAT | |
| | | | | CCCGGAGGTAAGTTGCAG | |
| | | | | CAGGGCGTCCCGGCAGCC | |
| | | | | GGCGGGCGATTGGTCGTA | |
| | | | | ATCCAGGATAAAGACGTG | |
| | | | | CATGGGACGGAGGCGTTT | |
| | | | | GGCCAAGACGTCCAAGG | |
| | | | | CCCAGGCAAACACGTTGT | |
| | | | | ACAGGTCGCCGTTGGGGG | |
| | | | | CCAGCAACTCGGGGGCCC | |
| | | | | GAAACAGGGTAAATAAC | |
| | | | | GTGTCCCCGATATGGGGT | |
| | | | | CGTGGGCCCGCGTTGCTC | |
| | | | | TGGGGCTCGGCACCCTGG | |
| | | | | GGCGGCACGGCCGTCCCC | |
| | | | | GAAAGCTGTCCCCAATCC | |
| | | | | TCCCGCCACGACCCGCCG | |
| | | | | CCCTGCAGATACCGCACC | |
| | | | | GTATTGGCAAGCAGCCCG | |
| | | | | TAAACGCGGCGAATCGCG | |
| | | | | GCCAGCATAGCCAGGTCA | |
| | | | | AGCCGCTCGCCGGGGCGC | |
| | | | | TGGCGTTTGGCCAGGCGG | |
| | | | | TCGATGTGTCTGTCCTCC | |
| | | | | GGAAGGGCCCCCAACAC | |
| | | | | GATGTTTGTGCCGGGCAA | |
| | | | | GGTCGGCGGGATGAGGG | |
| | | | | CCACGAACGCCAGCACG | |
| | | | | GCCTGGGGGGTCATGCTG | |
| | | | | CCCATAAGGTATCGCGCG | |
| | | | | GCCGGGTAGCACAGGAG | |
| | | | | GGCGGCGATGGGATGGC | |
| | | | | GGTCGAAGATGAGGGTG | |
| | | | | AGGGCCGGGGGCGGGGC | |
| | | | | ATGTGAGCTCCCAGCCTC | |
| | | | | CCCCCCGATATGAGGAGC | |
| | | | | CAGAACGGCGTCGGTCAC | |
| | | | | GGCATAAGGCATGCCCAT | |
| | | | | TGTTATCTGGGCGCTTGT | |
| | | | | CATTACCACCGCCGCGTC | |
| | | | | CCCGGCCGATATCTCACC | |
| | | | | CTGGTCGAGGCGGTGTTG | |
| | | | | TGTGGTGTAGATGTTCGC | |
| | | | | GATTGTCTCGGAAGCCCC | |
| | | | | CAGCACCTGCCAGTAAGT | |
| | | | | CATCGGCTCGGGTACGTA | |
| | | | | GACGATATCGTCGCGCGA | |
| | | | | ACCCAGGGCCACCAGCA | |
| | | | | GTTGCGTGGTGGTGGTTT | |
| | | | | TCCCCATCCCGTGAGGAC | |
| | | | | CGTCTATATAAACCCGCA | |
| | | | | GTAGCGTGGGCATTTTCT | |
| | | | | GCTCCAGGCGGACTTCCG | |
| | | | | TGGCTTCTTGCTGCCGGC | |
| | | | | GAGGGCGCAACGCCGTA | |
| | | | | CGTCGGTTGCTATGGCCG | |
| | | | | CGAGAACGCGCAGCCTG | |
| | | | | GTCGAACGCAGACGCGTG | |
| | | | | TTGATGGCAGGGGTACGA | |
| | | | | AGCCATAGGGCCGGGATT | |
| | | | | CTCCTCCACGTCACCAGC | |
| | | | | CTGCTTCAGCAGAGAGAA | |
| | | | | GTTGGTGGCTCCGCTTCC | |
| | | | | (133) | |
| RKB3719 | hPGK-Puro- | HSV-TK | F | CTTGTCATTACCACCGCC | mini-library |
| | P2A- | | | G (134) | intron |
| | HSV_TK | | | | amplification |
| RKB3720 | hPGK-Puro- | HSV-TK | R | CTTGTCATTACCACCGCC | Mini-library |
| | P2A- | | | G (135) | intron |
| | HSV_TK | | | | amplification |
| RKB3721 | hPGK-Puro- | HSV-TK | F | CGGCGGTGGTAATGACAA | amplify |
| | P2A- | | | G (136) | HSV-TK |
| | HSV_TK | | | | backbone |
| | | | | | for library |
| RKB3722 | hPGK-Puro- | HSV-TK | R | CCGATATCTCACCCTGGT | amplify |
| | P2A- | | | CG (137) | HSV-TK |
| | HSV_TK | | | | backbone |
| | | | | | for library |
| RKB4023 | hPGK-Puro- | HSV-TK | F | GGACGCGGCGGTGGTAAT | amplify |
| | P2A- | | | GACAAGCGCCCAGATAA | HSV-TK |
| | HSV_TK | | | CAATG (138) | backbone |
| | | | | | for main |
| | | | | | library |
| RKB4024 | hPGK-Puro- | HSV-TK | R | CCGGCCGATATCTCACCC | amplify |
| | P2A- | | | TGGTCGAGGCGGTGTTGT | HSV-TK |
| | HSV_TK | | | GTGG (139) | backbone |
| | | | | | for main |
| | | | | | library |
| RKB4032 | hPGK-Puro- | HSV-TK | F | CATTGTTATCTGGGCGCT | main library |
| | P2A- | | | TGTCATTACCACCGCCGC | intron oligo |
| | HSV_TK | | | GTCC (140) | and gDNA |
| | | | | | amplification |
| RKB4033 | hPGK-Puro- | HSV-TK | R | CCACACAACACCGCCTCG | main library |
| | P2A- | | | ACCAGGGTGAGATATCGG | intron oligo |
| | HSV_TK | | | CCGG (141) | and gDNA |
| | | | | | amplification |
| RKB3906 | hPGK-Puro- | HSV-TK/ | F | ACACTCTTTCCCTACACG | amplify |
| | P2A- | Illumina | | ACGCTCTTCCGATCTGAC | mini-library |
| | HSV_TK | landing | | CAGGGTGAGATATCG | with |
| | | site | | (142) | Illumina |
| | | | | | landing sites |
| | | | | | for MiSeq |
| RKB3907 | hPGK-Puro- | HSV-TK/ | R | GTGACTGGAGTTCAGACG | amplify |
| | P2A- | Illumina | | TGTGCTCTTCCGATCTTGT | mini-library |
| | HSV_TK | landing | | CATTACCACCGCC (143) | with |
| | | site | | | Illumina |
| | | | | | landing sites |
| | | | | | for MiSeq |
| RKB4188 | hPGK-Puro- | HSV-TK/ | F | ACACTCTTTCCCTACACG | amplify |
| | P2A- | Illumina | | ACGCTCTTCCGATCTNNN | mini-library |
| | HSV_TK | landing | | NNNNNNNNNGACCAGGG | with |
| | | site | | TGAGATATCG (144) | Illumina |
| | | | | | landing sites |
| | | | | | and NNN |
| | | | | | spacer for |
| | | | | | MiSeq |
| RKB4189 | hPGK-Puro- | HSV-TK/ | R | GTGACTGGAGTTCAGACG | amplify |
| | P2A- | Illumina | | TGTGCTCTTCCGATCTNN | mini-library |
| | HSV_TK | landing | | NNNNNNNNNNTTGTCATT | with |
| | | site | | ACCACCGCC (145) | Illumina |
| | | | | | landing sites |
| | | | | | and NNN |
| | | | | | spacer for |
| | | | | | MiSeq |
| NA | hPGK-Puro- | gBlock | gBlock | TCGACCAGGGTGAGATAT | synMTERF |
| | P2A- | | | CGGCCGGGTGAGTCGCCC | D3i1-150 |
| | HSV_TK | | | CCTTCCTCTGCTCCGAGA | |
| | | | | GGGAAATGGGAATTAGG | |
| | | | | GTGGTGGCAGAGCCCAA | |
| | | | | AGAGGCCTTGTAGGCCAT | |
| | | | | GGTAAGGCATTTCTATGT | |
| | | | | TTTATTTTACTTTGTCTTT | |
| | | | | ATCCTAAAATGCCATTGG | |
| | | | | CAAGTTTATTGCAGGGAC | |
| | | | | GCGGCGGTGGTAATGACA | |
| | | | | AGC (183) | |
| NA | hPGK-Puro- | gBlock | gBlock | AACATCTACACCACACAA | synMTERFD3i1- |
| | P2A- | | | CACCGCCTCGACCAGGGT | 100 |
| | HSV_TK | | | GAGATATCGGCCGGGTGA | |
| | | | | GTCGCCCCCTTAGGCCTT | |
| | | | | GTAGGCCATGGTAAGGCA | |
| | | | | TTTCTATGTTTTATTTTAC | |
| | | | | TTTGTCTTTATCCTAAAAT | |
| | | | | GCCATTGGCAAGTTTATT | |
| | | | | GCAGGGACGCGGCGGTG | |
| | | | | GTAATGACAAGCGCCCAG | |
| | | | | ATAACAATGGGCATGCCT | |
| | | | | T (184) | |
| NA | hPGK-Puro- | gBlock | gBlock | TCGACCAGGGTGAGATAT | synMTERFD3i1- |
| | P2A- | | | CGGCCGGCCGAGTCGCCC | 150, 5' |
| | HSV_TK | | | CCTTCCTCTGCTCCGAGA | and canon. |
| | | | | GGGAAATGGGAATTAGG | 3' ss |
| | | | | GTGGTGGCAGAGCCCAA | mutated |
| | | | | AGAGGCCTTGTAGGCCAT | |
| | | | | GGTAAGGCATTTCTATGT | |
| | | | | TTTATTTTACTTTGTCTTT | |
| | | | | ATCCTAAAATGCCATTGG | |
| | | | | CAAGTTTATTGCCCGGAC | |
| | | | | GCGGCGGTGGTAATGACA | |
| | | | | AGC (185) | |
| NA | hPGK-Puro- | gBlock | gBlock | CCAGGGTGAGATATCGGC | synMTERFD3i1- |
| | P2A- | | | CGGGTGAGTCGCCCCCTT | 150, |
| | HSV_TK | | | CCTCTGCTCCGAGAGGGA | inserted |
| | | | | AATGGGAATTAGGGTGGT | ideal |
| | | | | GGCAGAGCCCAAAGAGG | branchpoint |
| | | | | GCGGCGGTGGTAATGACA | |
| | | | | AGC (188) | |
| NA | hPGK-Puro- | gBlock | gBlock | GTGAGTCGCCCCCTTCCT | synMTERFD3i1- |
| | P2A- | | | CTGCTCCGAGAGGGAAAT | 150, |
| | HSV TK | | | GGGAATTAGGGTGGTGGC | branchpoints |
| | | | | AGAGCCCAAAGAGGCCTT | mutated |
| | | | | GTAGGCCATGGTAAGGCG | |
| | | | | TTTCTGTGTTTTGTTTTGC | |
| | | | | TTTGTCTTTATCCTAAAAT | |
| | | | | GCCATTGGCAAGTTTATT | |
| | | | | GCAG (189) | |

### Example 2

Example 1 demonstrated the design and successful implementation of a synthetic intron to implement expression of a transgene specifically in cells (e.g., cancer cells) with aberrant RNA splicing and delivery of this construct via lentivirus. This Example describes the design and use of an adeno-associated virus (AAV)-based vector construct to successfully deliver a transgene to cells and selectively express either (1) IL-2 alone, or (2) HSV-TK and IL-2 simultaneously in cells with aberrant RNA splicing (e.g., a mutation in SF3B1).

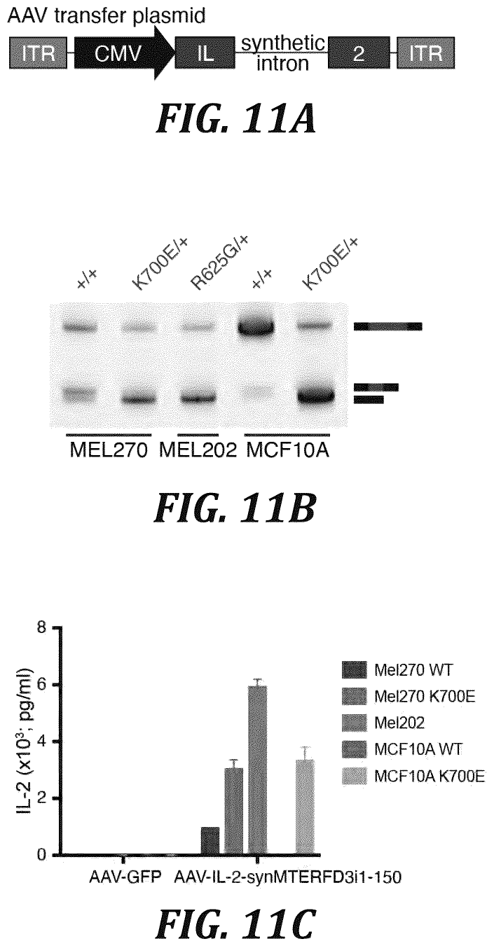

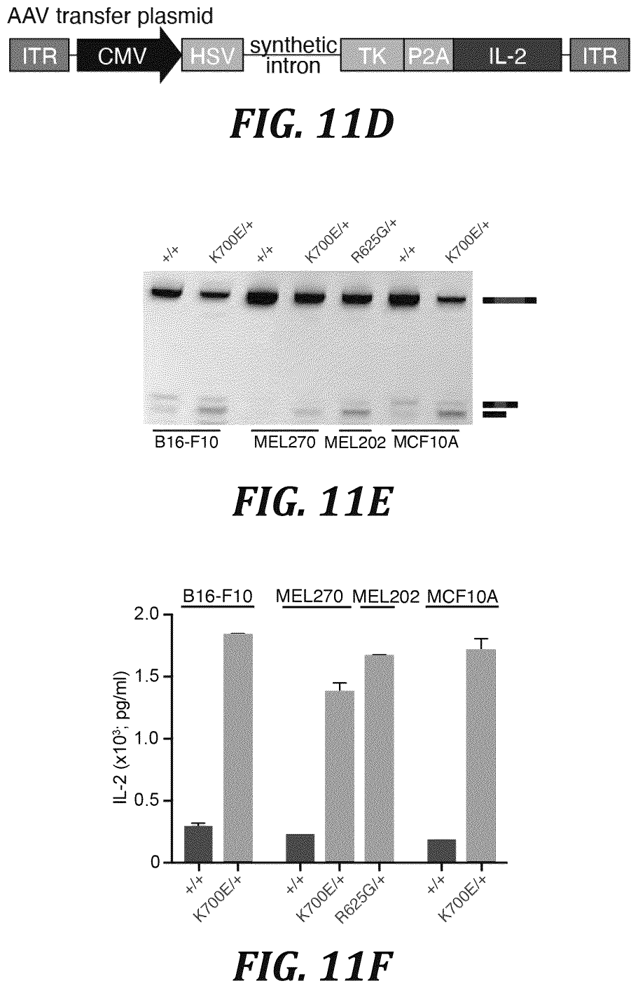

Specifically, FIG. 11A provides a schematic diagram of an AAV transfer plasmid with the gene encoding IL-2 interrupted by the synMTERFD3i1-150 synthetic intron. This transfer plasmid was used for AAV2-mediated delivery of the illustrated IL-2 construct to MEL270, MEL202, and MCF10A cells (2,000 vg/cell). FIG. 11B illustrates RT-PCR amplicons illustrating SF3B1 mutation-dependent splicing of the construct described in FIG. 11A following AAV2-mediated delivery to the indicated cells. FIG. 1C is a bar plot illustrating results of ELISA assay for IL-2 following AAV2-mediated delivery of a negative control (AAV-GFP) or the construct shown in FIG. 11A (AAV-IL-2-synMTERFD3i1-150). As illustrated, IL-2 was specifically expressed by cells with SF3B1 mutation-dependent splicing that received the construct.

In view of this success, an alternative vector construct was investigated wherein multiple CDSs could be expressed from a single construct with a synthetic intron. Specifically, FIG. 11D provides a schematic of an AAV transfer plasmid with HSV-TK interrupted by the synMTERFD3i1-150 synthetic intron, followed by P2A+IL-2. In this construct, HSV-TK and IL-2 proteins are produced only when the synthetic intron is spliced out. This transfer plasmid was used for AAV2-mediated delivery of the illustrated HSV-TK+IL-2 construct to B16-F10, MEL270, MEL202, and MCF10A cells (2,000 vg/cell). FIG. 11E illustrates RT-PCR results for SF3B1 mutation-dependent splicing of the construct in (FIG. 11D) following AAV2-mediated delivery to the indicated cells. FIG. 11F is a bar plot illustrating results of an ELISA assay for IL-2 following AAV2-mediated delivery of the construct shown in FIG. 11D. As illustrated, IL-2 was specifically expressed by cells with SF3B1 mutation-dependent splicing that received the construct.

These results demonstrate that the disclosed synthetic introns can be successfully integrated into expression vectors (e.g., viral vectors) for delivery to cell types of interest and achieve selective expression in cells with aberrant RNA splicing (e.g., a mutation in SF3B1). Additionally, the results indicate that this platform is applicable to designs wherein a synthetic intron is used to control expression of multiple proteins, as an alternative to expression of a single protein, to implement controlled and conditional expression of the transgenes in the target cells.

### Example 3

This Example discloses additional embodiments of the synthetic intron platform and the applicability to screening assays to detect cells with aberrant RNA splicing and distinguish between cells without or with aberrant RNA splicing.

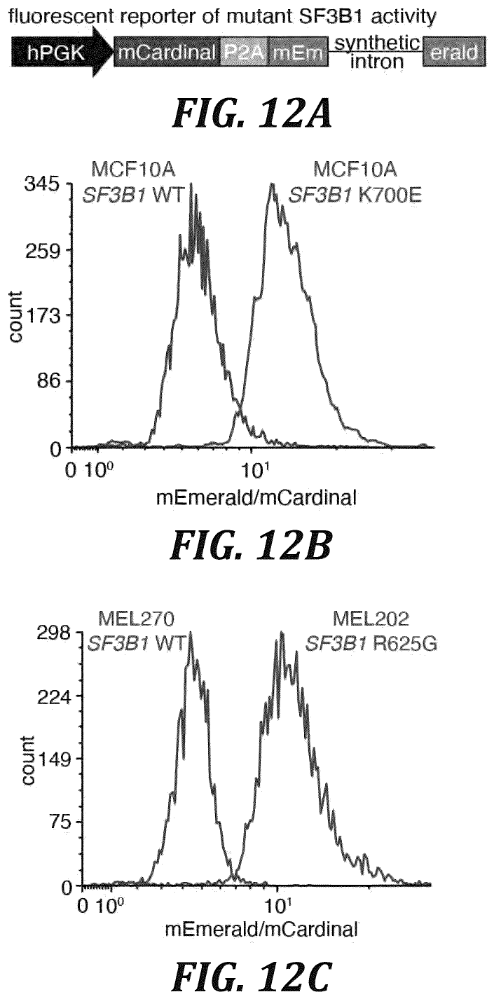

Specifically, reporter constructs were generated that implement fluorescent signals conditional upon aberrant RNA splicing (e.g., with mutation in the gene encoding SF3B1). FIG. 12A is a schematic of a fluorescent reporter construct with Emerald interrupted by the synMTERFD3i1-150 synthetic intron. mCardinal is used in the construct to provide a positive control signal. The construct was introduced to MCF10A cells with either wild-type SF3B1 or mutated SF3B1 (K700E substitution), MEL270 cells with wild-type SF3B1, or MEL202 cells with mutated SF3B1 (R625G substitution). FIG. 12B shows flow cytometry density plots illustrating the ratio of mEmerald to mCardinal following delivery of the reporter in FIG. 6A to the MCF10A cells. FIG. 12C shows flow cytometry density plots illustrating the ratio of mEmerald to mCardinal following delivery of the reporter in FIG. 12A to the MEL270 and MEL202 cells.

These results indicate a significant increase in conditional Emerald signal only in cells that contain SF3B1 mutations for both breast epithelial cells (MCF10A) and uveal melanoma cells (MEL270 and MEL202). Accordingly, the disclosed constructs can be used to distinguish cells with aberrant RNA splicing due to mutant SF3B1 activity. Such constructs are suitable for high-throughput screening of cells, for example, to screen for compositions and agents that antagonize mutations in the RNA splicing machinery (e.g., mutations in SF3B1).

While illustrative embodiments have been illustrated and described, it will be appreciated that various changes can be made therein without departing from the spirit and scope of the invention.

The embodiments of the invention in which an exclusive property or privilege is claimed are defined as follows:

1. An artificial nucleic acid intron construct, comprising an intron comprising:
a 5′ splice site;
a canonical 3′ splice site;
at least one cryptic 3′ splice site, that is within about 100 nucleotides upstream of the canonical 3′ splice site or within about 50 nucleotides downstream of the canonical 3′ splice site;
a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and
at least one branchpoint at least 15 nucleotides upstream of the canonical 3′ splice site.

2. The artificial nucleic acid intron construct of claim 1, wherein the intron is at least about 50 nucleotides to about 1000 nucleotides in length.

3. The artificial nucleic acid intron construct of claim 1, wherein the intron is derived from a human wildtype intron selected from intron 1 of MTERFD3, intron 4 of MYO15B, intron 10 of SYTL1, intron 11 of SYTL1, intron 4 of MAP3K7, intron 1 of ORAI2, and intron 1 of TMEM14C.

4. The artificial nucleic acid intron construct of claim 3, wherein the human wildtype intron from which the intron is derived is one of the following:
intron 1 of MTERFD3 comprising a sequence set forth in SEQ ID NO:2;
intron 4 of MYO15B comprising a sequence set forth in SEQ ID NO: 8;
intron 10 of SYTL1 comprising a sequence set forth in SEQ ID NO: 13;
intron 11 of SYTL1 comprising a sequence set forth in SEQ ID NO: 15;
intron 4 of MAP3K7 comprising a sequence set forth in SEQ ID NO: 22;
intron 1 of ORAI2 comprising a sequence set forth in SEQ ID NO:26; and
intron 1 of TMEM14C comprising a sequence set forth in SEQ ID NO:30.

5. The artificial nucleic acid intron construct of claim 3 or claim 4, wherein the intron is derived from a human wildtype intron 1 of MTERFD3, and wherein the intron further comprises one, two, three, or more of the following features:
a 5′ splice site comprising a GT dinucleotide immediately followed by a consensus 5′ splice site context, optionally wherein the consensus 5′ splice site context includes one of AAG, GAG, GTG, and the like;
a canonical 3′ splice site comprising an AG dinucleotide immediately preceded by a C or T;
at least one cryptic 3′ splice site, located at least 5 nucleotides upstream of the canonical 3′ splice site, with an AG dinucleotide and comprising a sequence that is a weaker 3′ splice site than is the canonical 3′ splice site, where splice site strength is estimated with the MaxEntScan algorithm or similar methods;
a pyrimidine-rich domain comprising at least 15 consecutive nucleotides, wherein the sequence of the pyrimidine-rich domain is at least 60% pyrimidine nucleotides and at least 40% thymine nucleotides, and wherein the pyrimidine-rich domain is within at least 30 nucleotides of a cryptic 3′ splice site; and
at least one branchpoint at least 20 nucleotides upstream of the canonical 3′ splice site.

6. The artificial nucleic acid intron construct of claim 4, wherein the intron has a 5′ end domain with about 10 to about 150 nucleotides having at least 50% sequence identity to a sequence of the 5′-most 10 to about 150 nucleotides of the wildtype intron.

7. The artificial nucleic acid intron construct of claim 4, wherein the intron has a 3′ end domain with about 50 to about 350 nucleotides having at least 50% sequence identity to a sequence of the 3′-most 50 to about 350 nucleotides of the wildtype intron.

8. The artificial nucleic acid intron construct of claim 4, wherein the intron has a sequence with at least 75% sequence identity to a sequence selected from SEQ ID NOS:4-6, 10, 11, 17-20, 24, 28, 32, and 150-157.

9. The artificial nucleic acid intron construct of claim 1, wherein the 5′ splice site comprises a sequence selected from GTGAG, GTAAG, GTGCG, GTACG, GTGGG, GTAGG, GTGTG, GTATG, and GTATC.

10. The artificial nucleic acid intron construct of claim 1, wherein the canonical 3′ splice site comprises a sequence selected from AAG, CAG, and TAG.

11. The artificial nucleic acid intron construct of claim 1, wherein the at least one cryptic 3′ splice site comprises a sequence selected from AAG, CAG, GAG, TAG, ATG, CTG, GTG, and TTG.

12. The artificial nucleic acid intron construct of claim 1, wherein the intron comprises a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites comprises a sequence independently selected from AAG, CAG, GAG, TAG, ATG, CTG, GTG, and TTG.

13. The artificial nucleic acid intron construct of claim 1, wherein the pyrimidine-rich domain is characterized by one, two, three, or all of the following:
wherein the pyrimidine-rich domain comprises at least 15 consecutive nucleotides;
wherein the pyrimidine-rich domain has a sequence with at least 60% pyrimidine nucleotides and is at least 40% thymine nucleotides;
wherein the pyrimidine-rich domain is within at least 30 nucleotides of a cryptic 3′ splice site; and
wherein the pyrimidine-rich domain has a sequence with at least 50% sequence identity to any 20 nucleotides selected from the sequence set forth as SEQ ID NO:49.

14. The artificial nucleic acid intron construct of claim 1, wherein the at least one branchpoint is at least 20 nucleotides upstream of the canonical 3′ splice site, and wherein the branchpoint nucleotide is an adenine.

15. The artificial nucleic acid intron construct of claim 1, wherein the branchpoint and surrounding sequence context has sequence identity of at least 60% to the sequence tactaAca, where the uppercase A is the branchpoint nucleotide.

16. The artificial nucleic acid intron construct of any one of claims 1-15, wherein the intron is configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene.

17. The artificial nucleic acid intron construct of claim 16, wherein the RNA splicing factor gene is SF3B1.

18. The artificial nucleic acid intron construct of claim 17, wherein the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190.

19. The artificial nucleic acid intron construct of any one of claims 1-18, further comprising a first exon domain and a second exon domain, wherein the intron is disposed between the first exon domain and the second exon domain.

20. The artificial nucleic acid intron construct of claim 19, wherein the combination of the first exon domain and the second exon domain without the intron encodes part or all of a protein of interest.

21. The artificial nucleic acid intron construct of claim 19 or claim 20, wherein the nucleic acid intron construct comprises an expression cassette comprising the first exon domain, the intron, the second exon domain, and a promoter sequence operatively linked thereto.

22. A method of generating an artificial nucleic acid intron construct with an intron, the method comprising:
(1) ligating a 5′ end domain of a human wildtype intron to a 3′ end domain of the human wildtype intron to provide an abbreviated intron that lacks an interior sequence, wherein the 5′ end domain comprises about 10 to about 150 nucleotides of the 5′ end sequence of the human wildtype intron, and wherein the 3′ end domain comprises about 50 to about 350 nucleotides of the 3′ end sequence of the human wildtype intron;
(2) implementing one or more sequence modifications to the abbreviated intron sequence to provide a first plurality of artificial introns derived from the abbreviated intron sequence;
(3) selecting artificial introns from the first plurality of artificial introns that conform to at least three of the following parameters:
a 5′ splice site;
a canonical 3′ splice site;
at least one cryptic 3′ splice site, that is within about 100 nt nucleotides upstream of the canonical 3′ splice site or within about 50 nt nucleotides downstream of the canonical 3′ splice site;
a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and
at least one branchpoint at least 15 nucleotides upstream of the canonical 3′ splice site.

23. The method of claim 22, wherein the human wildtype intron is selected from intron 1 of MTERFD3, intron 4 of MYO15B, intron 10 of SYTL1, intron 11 of SYTL1, intron 4 of MAP3K7, intron 1 of ORAI2, intron 1 of TMEM14C, or functional variants thereof.

24. The method of claim 23, wherein the human wildtype intron is one of the following:
intron 1 of MTERFD3 comprising a sequence set forth in SEQ ID NO:2;
intron 4 of MYO15B comprising a sequence set forth in SEQ ID NO: 8;
intron 10 of SYTL1 comprising a sequence set forth in SEQ ID NO: 13;
intron 11 of SYTL1 comprising a sequence set forth in SEQ ID NO: 15;
intron 4 of MAP3K7 comprising a sequence set forth in SEQ ID NO: 22;
intron 1 of ORAI2 comprising a sequence set forth in SEQ ID NO:26; and
intron 1 of TMEM14C comprising a sequence set forth in SEQ ID NO:30.

25. The method of claim 22, wherein the one or more sequence modifications comprises one or more of the following in any combination or order:
(a) mutating a single nucleotide;
(b) mutating any pair of nucleotides within 10 nucleotides of the 5′ end of the abbreviated intron sequence or 30 nucleotides of the 3′ end of the abbreviated intron sequence;
(c) deleting any consecutive stretch of 5, 10, 15, 20, 25, 30, 35, 40, 45, 50, 100, 125, 150, 200, or 250 nucleotides;
(d) mutating any pair of nucleotides within the 5 nt nucleotides upstream of and 2 nucleotides downstream of each branchpoint;
(e) mutating any combination of branchpoints to guanine;
(f) mutating any combination of multiple adenines to guanines;
(g) mutating any combination of branchpoint contexts to strong branchpoint contexts, optionally wherein the strong branchpoint context comprises a sequence with a sequence identity of at least 50% to the sequence tactaAca, where A is a branchpoint nucleotide and tacta_ca is a context sequence;
(h) mutating any four consecutive nucleotides to cAGg;
(i) inserting a polypyrimidine tract immediately followed by a 3′ splice site at any position;
j) mutating any consecutive stretch of nucleotides to one or more thymines;
(k) mutating all pyrimidines within any six or more consecutive positions to guanines;
(l) inserting a strong branchpoint and flanking sequence context at any position;
(m) inserting one or more intronic splicing enhancers at any position; and
(n) inserting one or more intronic splicing silencers at any position.

26. The method of claim 25, wherein the polypyrimidine tract immediately followed by a 3′ splice site comprises at least 6 consecutive nucleotides containing at least 4 pyrimidines, immediately followed by a sequence selected from AAG, CAG, GAG, TAG, ATG, CTG, GTG, or TTG, and the like.

27. The method of claim 25, wherein the strong branchpoint and flanking sequence context comprises a sequence with a sequence identity of at least 50% to the sequence tactaAca, where uppercase indicates the branchpoint, and the like.

28. The method of claim 25, wherein the one or more intronic splicing enhancers are selected from GGGTTT, GGTGGT, TTTGGG, GAGGGG, GGTATT, GTAACG, and the like.

29. The method of claim 25, wherein the one or more intronic splicing silencers are selected from CACACCA, CTCCTC, TACAGCT, CTTCAG, GAACAG, CAAAGGA, AGATATT, ACATGA, AATTTA, AGTAGG, and the like.

30. An artificial nucleic acid intron construct produced by the method of any one of claims 22-29.

31. A method of modifying a nucleic acid sequence to permit selective expression, or alternately selective lack of expression, in a cell characterized by a mutation in an RNA splicing factor gene, the method comprising:
(1) providing a sequence of a target nucleic acid molecule and sequence of an artificial nucleic acid intron as recited in one of claims 1-18 and 30, wherein the artificial nucleic acid intron is derived from a wildtype intron with known nucleotide sequences of upstream and downstream flanking exons;
(2) identifying one or more dinucleotides in the target nucleic acid sequence that are identical to an intron dinucleotide sequence consisting of the 3′-most nucleotide of the upstream exon flanking the wildtype intron and the 5′-most nucleotide of the downstream exon flanking the wildtype intron;
(3) selecting a dinucleotide identified in step (2) as an insertion point, wherein the insertion point divides the target nucleic acid into a first domain and a second domain, optionally wherein one of the first domain and second domain is at least about 50% of the length of the other of the first domain and second domain; and
(4) inserting an artificial intron molecule with the artificial nucleic acid intron sequence between the first domain and the second domain of the target nucleic acid molecule.

32. The method of claim 31, wherein step (3) further comprises:
computationally inserting the sequence of the artificial nucleic acid intron at the selected insertion point to create a hypothetical exonic flanking sequence context for a 5′ splice site and a 3′-most 3′ splice site;
computing strength scores for the 5′ splice site and the 3′-most 3′ splice site, respectively, in their hypothetical exonic contexts;
comparing the computed strength scores for the 5′ splice site and 3′-most 3′ splice site within their hypothetical exonic contexts to strength scores of the respective 5′ splice site and 3′-most 3′ splice site of the wildtype intron in its wildtype exonic context from which the artificial nucleic acid intron is derived; and
selecting a dinucleotide wherein computational insertion of the artificial nucleic acid intron sequence results in strength scores for the 5′ splice site and 3′-most 3′ splice site in their hypothetical exonic contexts that differ by about 50% or less of the respective 5′ splice site and 3′-most 3′ splice site scores of the wildtype intron in its wildtype exonic context.

33. The method of claim 32, wherein strength scores are computed with a standard method such as MaxEntScan::scores5ss, MaxEntScan::score3ss, HumanSplicingFinder, and other similar algorithms.

34. The method of claim 32, further comprising introducing one or more synonymous codon mutations into the nucleic acid that improve or weaken one or both scores for the 5′ splice site and/or 3′-most 3′ splice site in their hypothetical exonic contexts.

35. The method of claim 31, further comprising introducing one or more synonymous codon mutations into the nucleic acid that result in creation of one or more exonic splicing enhancers.

36. The method of claim 35, wherein the one or more exonic splicing enhancers is/are selected from CCNG, CGNG, GCNG, and GGNG, where N is any nucleotide, and other sequences with enhanced likelihood of binding by serine/arginine-rich (SR) proteins.

37. The method of claim 31, further comprising introducing one or more synonymous codon mutations into the nucleic acid that result in creation of one or more exonic splicing silencers.

38. The method of claim 37, wherein the one or more exonic splicing silencers is/are selected from TTTGTTCCGT (SEQ ID NO:160), GGGTGGTTTA (SEQ ID NO:161), GTAGGTAGGT (SEQ ID NO:162), TTCGTTCTGC (SEQ ID NO:163), GGTAAGTAGG (SEQ ID NO:164), GGTTAGTTTA (SEQ ID NO:165), TTCGTAGGTA (SEQ ID NO:166), GGTCCACTAG (SEQ ID NO:167), TTCTGTTCCT (SEQ ID NO:168), TCGTTCCTTA (SEQ ID NO:169), GGGATGGGGT (SEQ ID NO:170), GTTTGGGGGT (SEQ ID NO:171), TATAGGGGGG (SEQ ID NO:172), GGGGTTGGGA (SEQ ID NO:173), TTTCCTGATG (SEQ ID NO:174), TGTTTAGTTA (SEQ ID NO:175), TTCTTAGTTA (SEQ ID NO:176), GTAGGTTTG, GTTAGGTATA (SEQ ID NO:177), TAATAGTTTA (SEQ ID NO:178), TTCGTTTGGG (SEQ ID NO:179), and the like, or sequences with at least 50% identity thereto.

39. The method of one of claims 31-38, wherein two or more artificial intron molecules are inserted into the target nucleic acid resulting in a plurality of domains, optionally wherein each of the plurality of domains is at least about 50% of the length of the other domain(s).

40. The method of one of claims 31-39, wherein the target nucleic acid molecule is an isolated nucleic acid molecule with a protein-coding sequence (CDS) that encodes a protein of interest, and the modified target nucleic acid molecule is configured to permit selective expression, or alternately selective lack of expression, in a cell characterized by a mutation in an RNA splicing factor gene.

41. The method of claim 40, further comprising introducing the modified target nucleic acid molecule to a cancer cell with a mutation in an RNA splicing factor gene and permitting expression, or alternately selective lack of expression, of the protein of interest.

42. The method of one of claims 31-39, wherein the target nucleic acid molecule is a gene in the chromosome of a cell, wherein the gene encodes a protein of interest, and the modified target nucleic acid molecule is configured for selective expression, or alternately selective lack of expression, in a cell characterized by a mutation in an RNA splicing factor gene.

43. The method of one of claims 31-42, wherein the cell is a cancer cell and the mutation in an RNA splicing factor gene is a change-of-function or loss-of-function mutation in a recurrently mutated RNA splicing factor gene;
wherein the artificial intron sequence is configured to be spliced differently in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene;
wherein the different splicing pattern of the artificial intron sequence results in production of different mature transcripts of the modified target nucleic acid molecule in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; and
wherein the production of different mature transcripts of the modified nucleic acid molecule permits either selective expression, or alternately selective lack of expression, of a desired protein from the target nucleic acid molecule in the cancer cell, and the opposite pattern in a cell lacking the change-of-function or loss-of-function mutation in the recurrently mutated RNA splicing factor gene.

44. The method of claim 43, wherein the RNA splicing factor gene is SF3B1.

45. The method of claim 44, wherein the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO: 190.

46. A method of selectively expressing, or alternately selectively not expressing, a gene of interest in a cell, wherein the cell comprises a change-of-function or loss-of-function mutation in a recurrently mutated RNA splicing factor gene, the method comprising:
introducing to the cell an expression cassette comprising a coding sequence (CDS) interrupted by at least one artificial nucleic acid intron as recited in one of claims 1-18 and 30, wherein the expression cassette further comprises a promoter operatively linked to the CDS; and
permitting transcription of the coding sequence and modified splicing of the transcript induced by the artificial nucleic acid intron in the resulting transcript in conjunction with the mutated splicing factor.

47. The method of claim 46, wherein the cell is a cancer cell and the mutation in an RNA splicing factor gene is a change-of-function or loss-of-function mutation in a recurrently mutated RNA splicing factor gene.

48. The method of claim 47, wherein the RNA splicing factor gene is SF3B1.

49. The method of claim 48, wherein the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190.

50. The method of one of claims 47-49, wherein the cancer is a myelodysplastic syndrome (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, or other neoplasm with recurrent SF3B1 mutations.

51. The method of one of claims 47-50, wherein upon splicing of the at least one artificial nucleic acid intron from the gene transcript the gene of interest encodes a functional therapeutic protein.

52. The method of claim 51, wherein the functional therapeutic protein is a toxin, chemokine, cytokine, growth factor, targetable cell-surface protein, targetable antigen, druggable enzyme, detectable marker, and the like.

53. A method of treating in a subject with cancer, wherein the cancer is characterized by a change-of-function or loss-of-function mutation in a recurrently mutated RNA splicing factor gene, the method comprising:
administering to the subject an effective amount of a therapeutic composition comprising an expression cassette comprising a coding sequence (CDS) interrupted by at least one artificial nucleic acid intron as recited in one of claims 1-18 and 30, wherein the expression cassette further comprises a promoter operatively linked to the CDS.

54. The method of claim 53, wherein the RNA splicing factor gene is SF3B1.

55. The method of claim 54, wherein the recurrent change-of-function mutation in SF3B1 resulting in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190.

56. The method of one of claims 53-55, wherein the cancer is selected from a myelodysplastic syndromes (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, and other neoplasm with recurrent SF3B1 mutations.

57. The method of one of claims 53-56, wherein upon splicing of the at least one artificial nucleic acid intron from the gene transcript in a cancer cell the CDS encodes a functional therapeutic protein.

58. The method of claim 57, wherein the functional therapeutic protein is a toxin, chemokine, cytokine, growth factor, targetable cell-surface protein, targetable antigen, druggable enzyme, detectable marker, and the like.

59. The method of claim 58, wherein the functional therapeutic protein is a chemokine, cytokine, or growth factor, and wherein the chemokine, cytokine, or growth factor stimulates an increased immune response against the cancer cell.

60. The method of claim 59, wherein the functional therapeutic protein is IFN alpha, IFN beta, IFN-gamma, IL-2, IL-12, IL-15, IL-18, IL-24, TNF-alpha, GM-CSF, and the like, or functional domains or derivatives thereof.

61. The method of claim 58, wherein the functional therapeutic protein is a targetable cell-surface protein or targetable antigen, and the method further comprises administering to the subject an effective amount of a second therapeutic composition comprising an affinity reagent that specifically binds the antigen.

62. The method of claim 61, wherein the targetable cell-surface protein or targetable antigen is CD19, CD22, CD23, CD123, ROR1, truncated EGFR (EGFRt), or functional domains thereof, and the like.

63. The method of claim 61, wherein the second therapeutic composition comprises an antibody, or a fragment or derivative thereof, an immune cell expressing an antibody, or fragment or derivative thereof, or an immune cell expressing a T cell receptor, or fragment or derivative thereof, and wherein the antibody or T cell receptor, or fragment or derivative thereof, specifically binds the antigen.

64. The method of claim 58, wherein the functional therapeutic protein is a toxin, wherein the toxin is optionally Caspase 9, TRAIL, Fas ligand, and the like, or functional fragments thereof.

65. The method of claim 58, wherein the functional therapeutic protein is a druggable enzyme, optionally wherein:
the druggable enzyme is herpes simplex virus thymidine kinase and the method further comprises administering to the subject an effective amount of ganciclovir;
the druggable enzyme is cytosine deaminase and the method further comprises administering to the subject an effective amount of 5-fluorocytosine;
the druggable enzyme is nitroreductase and the method further comprises administering to the subject an effective amount of CB1954 or analogs thereof;
the druggable enzyme is carboxypeptidase G2 and the method further comprises administering to the subject an effective amount of CMDA, ZD-2767P, and the like;
the druggable enzyme is purine nucleoside phosphorylase and the method further comprises administering to the subject an effective amount of 6-methylpurine deoxyriboside, and the like;
the druggable enzyme is cytochrome P450 and the method further comprises administering to the subject an effective amount of cyclophosphamide, ifosfamide, and the like;
the druggable enzyme is horseradish peroxidase and the method further comprises administering to the subject an effective amount of indole-3-acetic acid, and the like; or
the druggable enzyme is carboxylesterase and the method further comprises administering to the subject an effective amount of irinotecan, and the like.

66. The method of claim 58, wherein the functional therapeutic protein is a detectable marker, and the method further comprises surgically removing the cancer cells expressing the detectable marker.

67. The method of one of claims 53-66, wherein the expression cassette is disposed in a vector, optionally a viral vector, for intracellular delivery.

68. The method of claim 67, wherein the viral vector is derived from AAV, adenovirus, herpes simplex virus, retrovirus, lentivirus, alphavirus, flavivirus, rhabdovirus, measles virus, Newcastle disease virus, Coxsackievirus, poxvirus, and the like.

69. The method of one of claims 53-67, wherein the therapeutic composition further comprises a vehicle for intracellular delivery and a pharmaceutically acceptable carrier.

70. The method of claim 69, wherein the vehicle is a liposome, nanocapsule, nanoparticle, exosome, microparticle, microsphere, lipid particle, vesicle, and the like, configured for the introduction of the expression cassette into cancer cells.

71. A method of enhancing surgical resection of a tumor from a subject, wherein the tumor is characterized by a change-of-function or loss-of-function mutation in a recurrently mutated RNA splicing factor gene, the method comprising:
administering to the subject an effective amount of a therapeutic composition comprising an expression cassette comprising a coding sequence (CDS) encoding a detectable marker, wherein the CDS is interrupted by at least one artificial nucleic acid intron as recited in one of claims 1-18 and 30, and wherein the expression cassette further comprises a promoter operatively linked to the CDS.

72. The method of claim 71, wherein the RNA splicing factor gene is SF3B1.

73. The method of claim 72, wherein the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, and combinations thereof, with reference to the wild-type amino acid sequence set forth in SEQ ID NO:190.

74. The method of one of claims 71-73, wherein the cancer is selected from a uveal melanoma, mucosal melanoma, skin melanoma, breast cancer, pancreatic cancer, endometrial cancer, liver cancer, lung cancer, mesothelioma, or other solid tumor or neoplasm with recurrent SF3B1 mutations.

75. The method of one of claims 71-74, wherein the detectable marker is a fluorescent or luminescent protein.

76. The method of claim 75, further comprising detecting fluorescent or luminescent tumor cells and surgically resecting the fluorescent or luminescent tumor cells.

77. The method of one of claims 71-76, wherein the expression cassette is disposed in a vector, optionally a viral vector, for intracellular delivery.

78. The method of claim 77, wherein the viral vector is derived from AAV, adenovirus, herpes simplex virus, retrovirus, lentivirus, alphavirus, flavivirus, rhabdovirus, measles virus, Newcastle disease virus, Coxsackievirus, poxvirus, and the like.

79. The method of claim 71-78, wherein the therapeutic composition further comprises a vehicle for intracellular delivery and a pharmaceutically acceptable carrier.

80. The method of claim 79, wherein the vehicle is a liposome, nanocapsule, nanoparticle, exosome, microparticle, microsphere, lipid particle, vesicle, and the like, configured for the introduction of the expression cassette into cancer cells.

81. An in vitro method of screening candidate compositions for activity in a cell, wherein the cell has a genetic background comprising a change-of-function or loss-of-function mutation in a recurrently mutated RNA splicing factor gene, the method comprising:
contacting the cell with an expression cassette comprising a coding sequence (CDS) interrupted by at least one artificial nucleic acid intron as recited in one of claims 1-18 and 30, wherein the expression cassette further comprises a promoter operatively linked to the CDS, and wherein upon splicing of the artificial nucleic acid intron the CDS encodes or does not encode a detectable reporter protein, wherein the specific splicing outcome depends upon mutant splicing factor activity in the cell;
contacting the cell with a candidate composition;
permitting transcription of the coding sequence; and
detecting the presence or absence of a functional reporter protein.

82. The method of claim 81, wherein detection of a functional reporter protein or a relative increase of functional reporter protein in the cell indicates the candidate composition does not suppress activity of the mutated RNA splicing factor in the cell, and wherein detection of an absence or relative reduction in functional reporter protein in the cell indicates the candidate composition does suppress activity of the mutated RNA splicing factor in the cell.

83. The method of claim 81, wherein detection of a functional reporter protein in the cell indicates the candidate composition suppresses activity of the mutated RNA splicing factor in the cell, and wherein an absence or relative reduction in detected functional reporter protein in the cell indicates the candidate composition does not suppress activity of the mutated RNA splicing factor in the cell.

84. The method of one of claims 81-83, wherein detecting the presence of a functional reporter protein comprises quantifying the amount of reporter protein.

85. The method of one of claims 81-84, wherein the reporter protein is a fluorescent or luminescent protein.

86. The method of one of claims 81-85, further comprising contacting a control cell without a change-of-function or loss-of-function mutation in a recurrently mutated RNA splicing factor gene with the expression cassette and further contacting the control cell with the candidate composition.

87. The method of claim 81, wherein the candidate composition is selected from a small molecule, protein (e.g., antibody, or fragment or derivative thereof, enzyme, and the like), and nucleic acid construct to alter the genome or transcriptome of the cell, or a complex of a nucleic acid and protein.

88. The method of claim 87, wherein the nucleic acid construct is an interfering RNA construct.

89. The method of claim 87, wherein the candidate composition comprises a guide nucleic acid specific for a target sequence and an associated nuclease that modifies and/or cleaves a nucleic acid molecule upon binding of the guide nucleic acid to its target sequence.

90. The method of claim 87, wherein the candidate composition comprises a guide nucleic acid specific for a target sequence and an associated catalytically inactive nuclease, wherein binding of the guide nucleic acid to the target sequence results in modification of transcription, splicing, or translation of the target sequence.

91. The method of claim 89 or claim 90, wherein the associated nuclease is Cas9, Cas12, Cas13, Cas14, variants thereof, and the like.

92. The method of claim 87, wherein the candidate composition comprises a Transcription Activator-Like Effector Nuclease (TALEN), Zinc Finger Nuclease (ZFN), or recombinase fusion protein.


SEQUENCE LISTING (190 sequences)

SEQ ID NO: 1 - DNA, 198 bp, Homo sapiens
gattggccatgggcgaagggactgatggcctggttggcgctgctgagaactgtcgggagcttcttgagaccgaggaccgaaatcccggctccaggcctcggggactgcggactgtggggaggctggccggagagagagggaaggacggggcctggcccccgggactccctgtgccttgcttggagctgacgccgacgg

SEQ ID NO: 2 - DNA, 1753 bp, Homo sapiens
gtgagtcgcccccttcctctgctcctgggcgtgttcctcaccagcggcgccgcagcggtcagggcccgcaaaaccccacgcctcgccagacgctcagctcagcctgccgggtttctctggaaaagcggaggccccacagtgaatgtgcggccttttgatcggtcctggagatcttccgtggaccctcgtttttccttgcttataaacgttggtcaatttgaaactggcagcagggcatatgtgttacgaaagaagaactttattgaaaccagtggtggtgtagagaagaccatactaaccataactgtgtattacagaagtgaatttcactccccaaaaatgcttaagaaacaagcatcctagaacagcccacattgtgatttaacaaacatttaccaaaagcattgctatatgtgggggcacagggctaagagcaggcaggactctggagccaacctgtggggtttgagtccaaattctgccggttttttgttttttgtttttttttttagctgagtaaccttgggcaagttcctttacctgcgtctgtttcctcattttaaaatgagttgttgtgatgatttaatgatttagtatatgtgaagagttttctgcatattctaagtgatttatggtattaactcgtttgatctttgcagtcacccaggagatagtatcaccattttcctgatgccaaactgaggctcagagaggtgaagtaacttcttcaagtcacaacacaactgttgtccaggaatcaggcagtctggctctgctaacccagttttacccactgcattaatgtaccgcatccacttgttcactcagtttcacctgacaaatactaattttgtctctctgtggaacaagttctttgctgggtgcagggaataaagcagaacaccgttcagttcctgccttcccaacgcttgtaaatctagtgcaggtcattcatggccgggctgcagggtgtccatgaatcctgctaaaatcatttgcgaaatgttgtgagcgtgggtcttttcttctggaaagagagtctgtgactctcaggttctgagttgatctgtgatccaaacagaggtgtgaagaagtgttgttttagcagatctgtgcctagtatgcctgatacacagcttgaacttcaaacaaacagcaacaaaattcctactggttggaagaatgaagaacttgtaagagcataataagtgctgcagtagcgtccggagcagggtgctgtggaagagcacacagaaggccacctgcccaggaatgcctcctagaggcagtgaaggcagctgggtgaaggatagggaagcagttcattggaacactgtggtgaccagtctgtccatgtagtgttgagacagaaacaacacgtgaacagctgcagctgttgtaccacttgtgcgcaagtgtaaaagtccatggtgccatgaaatcatgtaatggggacaggaggcttcatgtattcgtgaaggttagaaaagtttccttgagaaagtgacacttaggcctaaaagttggagcattttgggcatggaatctggcccacagtcttcaaagaccacatggtagaaaccagtagagcaccagggactgacaggaagcaaatgcagctggtgcaggagagggaaatgggaattagggtggtggcagagcccaaagaggccttgtaggccatggtaaggcatttctatgttttattttactttgtctttatcctaaaatgccattggcaagtttattgcag

SEQ ID NO: 3 - DNA, 101 bp, Homo sapiens
ggaactgacaagatcacattttgagaagaagttggaaagaatcccaagtggatgaactgaatatctggatgaggacaagatctgtggggagagactgtaag

SEQ ID NO: 4 - DNA, 250 bp, Artificial sequence
  Synthetic
gtgagtcgcccccttcctctgctcctgggcgtgttcctcaccagcggcgccgcagcggtcagggcccgcaaaaccccacgcctcgccagacgctcagctccaggaagcaaatgcagctggtgcaggagagggaaatgggaattagggtggtggcagagcccaaagaggccttgtaggccatggtaaggcatttctatgttttattttactttgtctttatcctaaaatgccattggcaagtttattgcag

SEQ ID NO: 5 - DNA, 150 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcag

SEQ ID NO: 6 - DNA, 100 bp, Artificial sequence
  Synthetic
gtgagtcgcccccttaggccttgtaggccatggtaaggcatttctatgttttattttactttgtctttatcctaaaatgccattggcaagtttattgcag

SEQ ID NO: 7 - DNA, 75 bp, Homo sapiens
acacatctttgccatcgtggcatcagcctatgacctggctcagaatactgggcaggacccatgcatcctcctgtg

SEQ ID NO: 8 - DNA, 369 bp, Homo sapiens
gtgagtggtggccttcctgggtggctgaacccatgggccacactgagggttgatggggcggggcctgaggtcttcactggaagccactggtatgctccactgctgggctacagttctggactcctcagggccgagctcctggctgaaatcatcacatgagcccctggaatgtggcatcttggggactttttcttttctgtcgggtctctccatgccctgggactatctttccacatttatgggggtctctccaggctagctgtcacttctgggtggcacatcccacttcctgggttcactggggtggtcctatgtgcccctccctggagaccctaccaaccaacacatccttagctcctcccactgcag

SEQ ID NO: 9 - DNA, 97 bp, Homo sapiens
tgggcacagtggctcagggaagacggaagccgccaaaaagatcatgcagttcctaagcagcctggagcaggatcagacggggaaccgagagtgtcag

SEQ ID NO: 10 - DNA, 350 bp, Artificial sequence
  Synthetic
gtgagtggtggccttcctgggtggctgaacccatgggccacactgagggtgtcttcactggaagccactggtatgctccactgctgggctacagttctggactcctcagggccgagctcctggctgaaatcatcacatgagcccctggaatgtggcatcttggggactttttcttttctgtcgggtctctccatgccctgggactatctttccacatttatgggggtctctccaggctagctgtcacttctgggtggcacatcccacttcctgggttcactggggtggtcctatgtgcccctccctggagaccctaccaaccaacacatccttagctcctcccactgcag

SEQ ID NO: 11 - DNA, 265 bp, Artificial sequence
  Synthetic
gtgagtggtggccttcctgggtggctgaacccatgggccacactgagggttgatggggcggggcctgaggtcttcactggaagccactggtatgctccactctctccatgccctgggactatctttccacatttatgggggtctctccaggctagctgtcacttctgggtggcacatcccacttcctgggttcactggggtggtcctatgtgcccctccctggagaccctaccaaccaacacatccttagctcctcccactgcag

SEQ ID NO: 12 - DNA, 97 bp, Homo sapiens
ctacgtcaaaagctacctcctcccggataagcagagcaagcgcaagacggcggtgaagaaacggaatctgaatccggttttcaacgagactctccgg

SEQ ID NO: 13 - DNA, 308 bp, Homo sapiens
gtgaggctgtgaccacgatgcggttccccgttaatgaactggacgcccccttcctgcggggctaggtggcaagggcagccagtaacgtcattgcccggaggatcggcggagggggcccattaactcgttatccagtgttgtcagcctcgtggtgggcgtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcag

SEQ ID NO: 14 - DNA, 159 bp, Homo sapiens
tactccgtcccgcaggccgagcttcagggccgcgtgctgagcctgtctgtgtggcaccgcgaaagcctgggtcgcaacatctttctgggcgaagttgaagtgcccctggacacgtgggactggggctctgagcccacctggctccccctgcagccccgg

SEQ ID NO: 15 - DNA, 305 bp, Homo sapiens
gtgaggcagccaggccgcgtggggagacctgcggcccgggtctcctgcatttaccccaccaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttctagccgcaccccatccgggtctgcagaccccaccctcctgaggcccctttccattagcccctgctccacgataagcccgcctctcgcag

SEQ ID NO: 16 - DNA, 79 bp, Homo sapiens
gtcccaccctctcccgacgaccttccgagccgcgggttactcgccctgtccctcaagtacgtccccgccggctccgagg

SEQ ID NO: 17 - DNA, 300 bp, Artificial sequence
  Synthetic
gtgaggctgtgaccacgatgcggttccccgttaatgaactggacgcccccgggctaggtggcaagggcagccagtaacgtcattgcccggaggatcggcggagggggcccattaactcgttatccagtgttgtcagcctcgtggtgggcgtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcag

SEQ ID NO: 18 - DNA, 250 bp, Artificial sequence
  Synthetic
gtgaggctgtgaccacgatgcggttccccgttaatgaactggacgcccccttcctgcggggctaggtggcaagggcagccagtaacgtcattgcccggagtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcag

SEQ ID NO: 19 - DNA, 300 bp, Artificial sequence
  Synthetic
gtgaggcagccaggccgcgtggggagacctgcggcccgggtgcatttaccccaccaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttctagccgcaccccatccgggtctgcagaccccaccctcctgaggcccctttccattagcccctgctccacgataagcccgcctctcgcag

SEQ ID NO: 20 - DNA, 250 bp, Artificial sequence
  Synthetic
gtgaggcagccaggcaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttcccatccgggtctgcagaccccaccctcctgaggcccctttccattagcccctgctccacgataagcccgcctctcgcag

SEQ ID NO: 21 - DNA, 46 bp, Homo sapiens
gtgtgtcttgtgatggaatatgctgaagggggctctttatataatg

SEQ ID NO: 22 - DNA, 1407 bp, Homo sapiens
gtgagtgtcattagacctgtctttatctagtggattaaaataatttgaaaaattttaatataaaccctaagttgtttaacattcttcacaatttgccgtcagagtcccaaaagggcataatttttaaaaatctgctaagaataaaaatggaaagataagattctataacattagtatgtgaaatttagaggtctatcaattttttaagtagatggtactttgcatttcattaaaagcatttaagtaaaagtttactttttaaatagattttaaaatatcttcagatttttttctttttgccctctcataactttgagcctcctcattttagaattctttaaccatatttcgatttcactctgctttattttttcttttttttcaaaaaaaaatgtgaaaactcacttcacacaaccttttatttgtagtaatgaaaaaaaactttgtgtagttacattggagaacagagttcttttaccctaaaaataccaaccatatgcagaaatgtattgtctcttgtagcaccagttatataaaacaaattgttacattttattttttaattagtattattttatgtaaacttttacttatatttactgcccatgtgttcatcattgtgatggatatctaataccccattgtcttatttgattgaactacttctggtttttagccattacagaaaatacaggtgcaaacatcttgatatacattgttttgtttctttctgtactgttctctgcctctctcttgtagtagtgtgccataagactgctcataatttatattactttataaaagtctttatctgtttgaaaaaatgtggtaatgtatattgattttacttcaatgtaggaataacatttaatagatgtactattaaatgttaatatcatggcaattatccactcaaaattagtagatattttcatcctttttatttagaagaaaaattaaaaggccaggcatggtggctcactcctgtaatcccagcactttgggaggccaaggcaggcagatcatgaggtcaagagatcaagaccatcctggccaacatggtgataccctatctctactaaaaatacaaaaattagctgggcgtggtggcacacgcctgtagtcccagctactcgggaggctgagccaggagaatcgcttgaacccaggaggtggagtttacagtgagccaagatcgcaccactgcactccagcctggcgacaaagtgagagtccgtctcaaaaaacaaaaagaaaaaagaaagtaaaaaattaaaagcaggactgatggagcatatgtatatgtagaaaaatacccagtcatgtgttctgatagtgcttgcattcacattgtgtcttttgttatatgcaataaagttctttttagtttgtgcctttctttcgcag

SEQ ID NO: 23 - DNA, 139 bp, Homo sapiens
tgctgcatggtgctgaaccattgccatattatactgctgcccacgcaatgagttggtgtttacagtgttcccaaggagtggcttatcttcacagcatgcaacccaaagcgctaattcacagggacctgaaaccaccaaa

SEQ ID NO: 24 - DNA, 250 bp, Artificial sequence
  Synthetic
gtgagtgtcattagacctgtctttatctagtggattaaaataatttgaaaaattttaatataaaccctaagttgtttaacattcttcacaatttgccgtcaaagaaagtaaaaaattaaaagcaggactgatggagcatatgtatatgtagaaaaatacccagtcatgtgttctgatagtgcttgcattcacattgtgtcttttgttatatgcaataaagttctttttagtttgtgcctttctttcgcag

SEQ ID NO: 25 - DNA, 132 bp, Homo sapiens
gggggaccccgcgcgaccggcggaaggagggagggggccgcgctcggcgccccggccgggccactgggccacaggccacgcggccacgcagtccgagcgggagccgagccgggcggggcgagggcagctccg

SEQ ID NO: 26 - DNA, 2563 bp, Homo sapiens
gtgagtgtggcggcgcgggggcctggcgcggggagcggggtccctgcggctgggggggccggaattggaccggggacgctcggcggggaaggggacgactgtaggctgcccgggcgcggtcactacccatccggggttggtgacctccaccccgcgggatccctggcggggtgcggcgcccagaggatcagggtcggcggcgcagccggttccggacaccggggcgtccctgggggagggacagggatgcagcctagcgcccctgggaggccccctcccagtgcgcctgctggagagagacactcgccattgccggtgccccagatgcttagaggaagtcaaactccaagaccgccctctggaaatgtgtccagtaggccagccaggaagaaaagctcatctctagagaggggatcgcgttattctacaagacctgttttctcggactgctcattaaagcaaaaaaaaaaaaaaaaaaaaaaaagcaaccccacaaggttcagtcccggagcatcctctagggtctgtgccacctgacagaagtacagggctccatcctgccccaccccaccccctgcctgggatccagtgtgaggcccagactctgtccctccccctgggccaccgggcctggcacacacagttggcattgggaacataaaggatggatttgaaccccctcctgggctgctccttcgagacctggagacctgtctcaagacccagctccaggcagacagacatcgtttcctccacaatcccacccactctggggacatctgcagggcctcaccgatggggcatgggaggcctggcggctcccaggaagccaccctcagccctcactgccctgagctcttcacctccactctcagacctgagcccagcccacccttactgatggggcatccctcttgttcacccttccttcctttggaggccaccaggctctcccttgcgtcccactcctgccagtcggccagccatttccctgggttgaactgtggtcccaaaccagaatgatctagcagtgcccagaccgttccactcaccaatttcttccattctcctgtggctggacgcccagtcatgctgcccagaccctctggctcattccacagccccaggctccaagtgtcttccacccacatctccctgccccctccaccccccaggtggcagccagtcttccctccatatctcctggacaaggagagccaccccacatgtacacctggagtggccttcccgtctctcggaatccttccatgcaggcagcatcttgtctttgaggaaaggtccctcttttttaaaattctttttagaggccaggcgcagtggttcatgcctataatctcagcactttgggaggccaaggtgggcggatcacttgagctcaggagttctagaccagcctggccaacatagcaaaaccctgtctctaccaaaaataccaaaaattagccaggtatggtggcacacacatgtagtcccagctactcgggtggttgaggcgggaggatcgcttgagcctgggaggtagaatttgcagggagctgagattgtgccactgcactccagccttggtaacacagcaggaccccatgtcaaaaaaaattatttttagagacagcgtcttactctgttgcctaggctggtctccagctcctggccgcaggtgatcctcccaccttggcctcacaaaacactgggattacaggcatgaggcactttggcatgcaaagggccgctctgtcccctccccggtgttacctgcaagcaccttttcctggccttggtgtgaggcagccagtcctctgctccctgccctccattgaagcctttagctcagtggatgaggattgcccccccctcttttttttttttttttttgagatggagtctcgctctgttgcccaggctggagtgcagtggtgtgctcttggctcactgcaacctctgccaccacacccagctatttttttttttttaagacagagtctcactgtgtcacccaggctggagtgccgcggtaggatctcagctcactgcaacctccgcctcctgggatcaggcaattctcctgcctcagcctcctgagtagctgggattacaggcgcccaccaccatgcccagttactttttgtatttttagaagagacaggtttccaccatgttgtccaggctagtctcgaactcctgacctctagtgatccgcccacctcagcctcccaaagatgacaggtgtgagctactgctcctgacccacatccccttaccttgctcccctactgggaggccatgaattctggcctctttcatcttggatctttaagtaccaagtgcgctaagcacctgtgtgatggaggagggaggacggagggagcaaaggttagcgtctcacccctccagtctctgtatactgcggtcatcagacctgcatgctcaattctgttaactatgggggtggggggagaggaaaaatgcagtttccaaattacagcattttctattatgtggagctaacctacggattgcagcgtttctctgaattctccccaag

SEQ ID NO: 27 - DNA, 109 bp, Homo sapiens
ggaaggaacgtcccagggatggaagtgcttggatgcggtgctgctggctgcggatgtgcgcaaggagatgggatggagagcctgagttggcattcgtataaatgacctg

SEQ ID NO: 28 - DNA, 250 bp, Artificial sequence
  Synthetic
gtgagtgtggcggcgcgggggcctggcgcggggagcggggtccctgcggctgggggggccggaattggaccggggacgctcggcggggaaggggacgactctctgtatactgcggtcatcagacctgcatgctcaattctgttaactatgggggtggggggagaggaaaaatgcagtttccaaattacagcattttctattatgtggagctaacctacggattgcagcgtttctctgaattctccccaag

SEQ ID NO: 29 - DNA, 327 bp, Homo sapiens
gccctgcaggcgctgcggacaggggaagcacagagattccccgccgcgttccctggactcaggagctcgccgcgatgccccgccccactctccacccgctgaatgcagggcgcatgctgctacttggcggctcaagccccgcccgcaccgtccccattctctgaccgcccctctcccggtacactgcgcaggcacaacagagccgctcccctctcctcgccccgccaccgggacggagagcgcccgccgctgcatttccggcgacacctcgcagtcattcctgcggcttgcgcgcccttgtagacagccggggccttcgtgagaccg

SEQ ID NO: 30 - DNA, 1328 bp, Homo sapiens
gtgcgagtatttggggattattcttattttctgccacttttaacttttagggattatttaggagtttcacggccgtctgcttttcgtccccccgattcagcgggccttcaggccgtgtggtcggtgcttttctcgttgggtattttctgcttttaaaagaagttgtggatgcgcggagcccctgggctcctgaggcaaaggcttacccatgtagcatagtgttgcctcgtttcttggtgattttcttgctccatctctttaaaagcctcccttactcggtgccgtctcgagttaagaactgtgggcaagatcccaagcccgctgcccttccctgttttatggaaacttaattctttttttttttttttttgtccttgagggagggacttgctctgtcgcccatgcttgagtgcagttgcctgaatatgtctcattgcagcctctgcctcctgggctcaagccatcctcccacttcagcctcccaagtagctgggatttacagtcgcatatcaccatgcgcgattaatttttttatattttttagtagacaatgggtttcactatgttggccaggctggtcttgaactcctaaccccaggtgatccgcccgcctcggcctcctaaagtactgggatgacaggcgtcagccactgcgcttggcctaattctttaaacagaataaacggggtatgcattgctttcatcttttggctcactggacacaggatactttctgtaagaaaatagaagctgtttttccaagggtgtagtgtcacatgtgaatatgaccactgtttccgtatattttatcctctcctactactgccctcctaacaagaactgtgagttggacgcagaagtttctaaaaaagttgagctttgaaattggctgttgcagcagggatgaaaagcaacacccctacctcccctcaaaagagacattaaagtagttggattaagggcacgggagtatttgcttttgaatttagtgataacatgggtagctgatgaaatgactaacacattccctgattttagagctggtcagtggatcttgctgagtttcccgtgggcctatgtgattaaaactgaggttttcatgacaatggtcagcatctgttcagggtttactaggtgctaagcacctttacatgtgacatccattgaatgctcactacagccccaggaaatcggtaccagtgttatcctcatggtacagtgaagaatactgagacttaggttgcgtagcttgcaggttggacacacttctttctgactgctggagagctgtgcttttaactacctctgatccagcttgttttctgcag

SEQ ID NO: 31 - DNA, 64 bp, Homo sapiens
gtgcaggcctggggtagtctcctgtctggacagagaagagaaaaatgcaggacactggctcagt

SEQ ID NO: 32 - DNA, 250 bp, Artificial sequence
  Synthetic
gtgcgagtatttggggattattcttattttctgccacttttaacttttagggattatttaggagtttcacggccgtctgcttttcgtccccccgattcagagccccaggaaatcggtaccagtgttatcctcatggtacagtgaagaatactgagacttaggttgcgtagcttgcaggttggacacacttctttctgactgctggagagctgtgcttttaactacctctgatccagcttgttttctgcag

SEQ ID NO: 33 - DNA, 348 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgag

SEQ ID NO: 34 - DNA, 369 bp, Artificial sequence
  Synthetic
ggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 35 - DNA, 344 bp, Herpes simplex virus
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccgg

SEQ ID NO: 36 - DNA, 784 bp, Herpes simplex virus
ggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaac

SEQ ID NO: 37 - DNA, 375 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgag

SEQ ID NO: 38 - DNA, 342 bp, Artificial sequence
  Synthetic
ctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 39 - DNA, 361 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctgg

SEQ ID NO: 40 - DNA, 356 bp, Artificial sequence
  Synthetic
tgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 41 - DNA, 337 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgagg

SEQ ID NO: 42 - DNA, 380 bp, Artificial sequence
  Synthetic
tgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 43 - DNA, 510 bp, Herpes simplex virus
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctg

SEQ ID NO: 44 - DNA, 618 bp, Herpes simplex virus
tgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaac

SEQ ID NO: 45 - DNA, 414 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctg

SEQ ID NO: 46 - DNA, 303 bp, Artificial sequence
  Synthetic
gggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 47 - DNA, 336 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgag

SEQ ID NO: 48 - DNA, 381 bp, Artificial sequence
  Synthetic
gtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 49 - DNA, 35 bp, Artificial sequence
  Synthetic
catttctatgttttattttactttgtctttatcct

SEQ ID NO: 50 - protein, 9 aa, Artificial sequence
  Synthetic
LAGLIDADG

SEQ ID NO: 51 - protein, 6 aa, Artificial sequence
  Synthetic
GIYYIG

SEQ ID NO: 52 - protein, 5 aa, Artificial sequence
  Synthetic
  MISC_FEATURE (3)..(3): Wherein X at position 3 is defined as either D or E
  MISC_FEATURE (4)..(4): Wherein X at position 4 is defined as any amino acid
PDXXK

SEQ ID NO: 53 - DNA, 188 bp, Artificial sequence
  Synthetic
gcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccagccgagccacatcgctcagacacc

SEQ ID NO: 54 - DNA, 21 bp, Artificial sequence
  Synthetic
acgcggcggtggtaatgacaa

SEQ ID NO: 55 - DNA, 21 bp, Artificial sequence
  Synthetic
ggccgatatctcaccctggtc

SEQ ID NO: 56 - DNA, 150 bp, Artificial sequence
  Synthetic
ccgagtcgcccccttcctctgctccgagagggaaatgggaattagggtggtggcagagcccaaagaggccttgtaggccatggtaaggcatttctatgttttattttactttgtctttatcctaaaatgccattggcaagtttattgccc

SEQ ID NO: 57 - DNA, 100 bp, Artificial sequence; identical to SEQ ID NO: 6
  Synthetic

SEQ ID NO: 58 - DNA, 150 bp, Artificial sequence; identical to SEQ ID NO: 5
  Synthetic

SEQ ID NO: 59 - DNA, 150 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcgg

SEQ ID NO: 60 - DNA, 150 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaaatgccattggcacctttattgcag

SEQ ID NO: 61 - DNA, 150 bp, Artificial sequence
  Synthetic
gtgagtcgcccccttcctctgctccgagagggaaatgggaattagggtggtggcagagcccaaagaggccttgtaggccatggtaaggcgtttctgtgttttgttttgctttgtctttatcctaaaatgccattggcaagtttattgcag

SEQ ID NO: 62 - DNA, 158 bp, Artificial sequence
  Synthetic
gtgagtcgcccccttcctctgctccgagagggaaatgggaattagggtggtggcagagcccaaagaggccttgtaggccatggtaaggctactaacaatttctatgttttattttactttgtctttatcctaaaatgccattggcaagtttattgcag

SEQ ID NO: 63 - DNA, 250 bp, Artificial sequence; identical to SEQ ID NO: 4
  Synthetic

SEQ ID NO: 64 - DNA, 150 bp, Artificial sequence; identical to SEQ ID NO: 56
  Synthetic

SEQ ID NO: 65 - DNA, 100 bp, Artificial sequence; identical to SEQ ID NO: 6
  Synthetic

SEQ ID NO: 66 - DNA, 150 bp, Artificial sequence; identical to SEQ ID NO: 5
  Synthetic

SEQ ID NO: 67 - DNA, 150 bp, Artificial sequence; identical to SEQ ID NO: 59
  Synthetic

SEQ ID NO: 68 - DNA, 150 bp, Artificial sequence; identical to SEQ ID NO: 60
  Synthetic

SEQ ID NO: 69 - DNA, 150 bp, Artificial sequence; identical to SEQ ID NO: 61
  Synthetic

SEQ ID NO: 70 - DNA, 158 bp, Artificial sequence; identical to SEQ ID NO: 62
  Synthetic

SEQ ID NO: 71 - DNA, 250 bp, Artificial sequence; identical to SEQ ID NO: 4
  Synthetic

SEQ ID NO: 72 - DNA, 20 bp, Artificial sequence
  Synthetic
tttttttttttttttttcag

SEQ ID NO: 73 - DNA, 20 bp, Artificial sequence
  Synthetic
acgacggcaactacaagacc

SEQ ID NO: 74 - DNA, 23 bp, Artificial sequence
  Synthetic
tgtagttgtactccagcttgtgc

SEQ ID NO: 75 - DNA, 22 bp, Artificial sequence
  Synthetic
tgtcttgtgatggaatatgctg

SEQ ID NO: 76 - DNA, 20 bp, Artificial sequence
  Synthetic
tccctgtgaattagcgcttt

SEQ ID NO: 77 - DNA, 19 bp, Artificial sequence
  Synthetic
actccctgtgccttgcttg

SEQ ID NO: 78 - DNA, 20 bp, Artificial sequence
  Synthetic
cattttgggcatggaatctg

SEQ ID NO: 79 - DNA, 29 bp, Artificial sequence
  Synthetic
atggactcattctatcttacagtctctcc

SEQ ID NO: 80 - DNA, 20 bp, Artificial sequence
  Synthetic
agtccgagcgggagccgagc

SEQ ID NO: 81 - DNA, 21 bp, Artificial sequence
  Synthetic
agcagcaccgcatccaagcac

SEQ ID NO: 82 - DNA, 20 bp, Artificial sequence
  Synthetic
gacacctcgcagtcattcct

SEQ ID NO: 83 - DNA, 21 bp, Artificial sequence
  Synthetic
gtagccaaagccaaaccaatg

SEQ ID NO: 84 - DNA, 20 bp, Artificial sequence
  Synthetic
gtggcatcagcctatgacct

SEQ ID NO: 85 - DNA, 20 bp, Artificial sequence
  Synthetic
tccaggctgcttaggaactg

SEQ ID NO: 86 - DNA, 20 bp, Artificial sequence
  Synthetic
aagctacctcctcccggata

SEQ ID NO: 87 - DNA, 20 bp, Artificial sequence
  Synthetic
cttcaacttcgcccagaaag

SEQ ID NO: 88 - DNA, 28 bp, Artificial sequence
  Synthetic
gtcagatcgctcaggtgtcgtgagcctc

SEQ ID NO: 89 - DNA, 44 bp, Artificial sequence
  Synthetic
cagaggttgattgtcgaggcgcttgatatcgaattcccactaag

SEQ ID NO: 90 - DNA, 32 bp, Artificial sequence
  Synthetic
cgacacctgagcgatctgacggttcactaaac

SEQ ID NO: 91 - DNA, 39 bp, Artificial sequence
  Synthetic
ccagcccaaggtcttgaggcagcggatctgacggttcac

SEQ ID NO: 92 - DNA, 19 bp, Artificial sequence
  Synthetic
gcctcaagaccttgggctg

SEQ ID NO: 93 - DNA, 20 bp, Artificial sequence
  Synthetic
gcctcgacaatcaacctctg

SEQ ID NO: 94 - DNA, 19 bp, Artificial sequence
  Synthetic
tgaagttcgagggcgacac

SEQ ID NO: 95 - DNA, 18 bp, Artificial sequence
  Synthetic
cctcggcgcgggtcttgt

SEQ ID NO: 96 - DNA, 20 bp, Artificial sequence
  Synthetic
tgaaccgcatcgagctgaag

SEQ ID NO: 97 - DNA, 18 bp, Artificial sequence
  Synthetic
ccagggtgtcgccctcga

SEQ ID NO: 98 - DNA, 18 bp, Artificial sequence
  Synthetic
gtgaagttcgagggcgac

SEQ ID NO: 99 - DNA, 18 bp, Artificial sequence
  Synthetic
ctcggcgcgggtcttgta

SEQ ID NO: 100 - DNA, 18 bp, Artificial sequence
  Synthetic
gggcacaagctggagtac

SEQ ID NO: 101 - DNA, 18 bp, Artificial sequence
  Synthetic
caggatgttgccgtcctc

SEQ ID NO: 102 - DNA, 18 bp, Artificial sequence
  Synthetic
ctgaagggcatcgacttc

SEQ ID NO: 103 - DNA, 18 bp, Artificial sequence
  Synthetic
ctcgatgcggttcaccag

SEQ ID NO: 104 - DNA, 18 bp, Artificial sequence
  Synthetic
ggcgacaccctggtgaac

SEQ ID NO: 105 - DNA, 18 bp, Artificial sequence
  Synthetic
ctcgaacttcacctcggc

SEQ ID NO: 106 - DNA, 16 bp, Artificial sequence
  Synthetic
catgccgagagtgatc

SEQ ID NO: 107 - DNA, 20 bp, Artificial sequence
  Synthetic
tacacgccacctcgacatac

SEQ ID NO: 108 - DNA, 20 bp, Artificial sequence
  Synthetic
tatgtcgaggtggcgtgtac

SEQ ID NO: 109 - DNA, 20 bp, Artificial sequence
  Synthetic
gagatccgactcgtctgagg

SEQ ID NO: 110 - DNA, 20 bp, Artificial sequence
  Synthetic
cctcagacgagtcggatctc

SEQ ID NO: 111 - DNA, 350 bp, Artificial sequence
  Synthetic
gatgcccttcagctcgatgcggttcaccagggtgtcgccctcgaacttcactgcgaaagaaaggcacaaactaaaaagaactttattgcatataacaaaagacacaatgtgaatgcaagcactatcagaacacatgactgggtatttttctacatatacatatgctccatcagtcctgcttttaattttttactttctttgacggcaaattgtgaagaatgttaaacaacttagggtttatattaaaatttttcaaattattttaatccactagataaagacaggtctaatgacactcaccctcggcgcgggtcttgtagttgccgtcgtccttgaagaagatggtgcgc

SEQ ID NO: 112 - DNA, 350 bp, Artificial sequence
  Synthetic
atgcccttcagctcgatgcggttcaccagggtgtcgccctcgaacttcacctgcagaaaacaagctggatcagaggtagttaaaagcacagctctccagcagtcagaaagaagtgtgtccaacctgcaagctacgcaacctaagtctcagtattcttcactgtaccatgaggataacactggtaccgatttcctggggctctgaatcggggggacgaaaagcagacggccgtgaaactcctaaataatccctaaaagttaaaagtggcagaaaataagaataatccccaaatactcgcacctcggcgcgggtcttgtagttgccgtcgtccttgaagaagatggtgcgct

SEQ ID NO: 113 - DNA, 350 bp, Artificial sequence
  Synthetic
gcggtgatatagaccttgtggctgttgtagttgtactccagcttgtgccccttggggagaattcagagaaacgctgcaatccgtaggttagctccacataatagaaaatgctgtaatttggaaactgcatttttcctctccccccacccccatagttaacagaattgagcatgcaggtctgatgaccgcagtatacagagagtcgtccccttccccgccgagcgtccccggtccaattccggcccccccagccgcagggaccccgctccccgcgccaggcccccgcgccgccacactcaccaggatgttgccgtcctccttgaagtcgatgcccttcagctcgatgcggt

SEQ ID NO: 114 - DNA, 350 bp, Artificial sequence
  Synthetic
gatgttgccgtcctccttgaagtcgatgcccttcagctcgatgcggttcactgaggacaggaggtgagctttagtggagcactgctggtggcccgcgtagatgctgcgcgagttggctgaggcaggtcctaccgcgttcctaccatgatggaattctggggagcccctaagggcattaatggggacctgcactatcaccactccgggcaatgacgttactggctgcccttgccacctagccccgcaggaagggggcgtccagttcattaacggggaaccgcatcgtggtcacagcctcacccagggtgtcgccctcgaacttcacctcggcgcgggtcttgtagttgccg

SEQ ID NO: 115 - DNA, 350 bp, Artificial sequence
  Synthetic
tccttgaagtcgatgcccttcagctcgatgcggttcaccagggtgtcgccctgcaataaacttgccaatggcattttaggataaagacaaagtaaaataaaacatagaaatgccttaccatggcctacaaggcctctttgggctctgccaccaccctaattcccatttccctctcctgcaccagctgcatttgcttcctggagctgagcgtctggcgaggcgtggggttttgcgggccctgaccgctgcggcgccgctggtgaggaacacgcccaggagcagaggaagggggcgactcacctcgaacttcacctcggcgcgggtcttgtagttgccgtcgtccttgaaga

SEQ ID NO: 116 - DNA, 350 bp, Artificial sequence
  Synthetic
agcttgtgccccaggatgttgccgtcctccttgaagtcgatgcccttcagctaaggatgtgttggttggtagggtctccagggaggggcacataggaccaccccagtgaacccaggaagtgggatgtgccacccagaagtgacagctagcctggagagacccccataaatgtggaaagatagtcccagggcatggagagagtggagcataccagtggcttccagtgaagacctcaggccccgccccatcaaccctcagtgtggcccatgggttcagccacccaggaaggccaccactcacctcgatgcggttcaccagggtgtcgccctcgaacttcacctcggcgcggg

SEQ ID NO: 117 - DNA, 14 bp, Artificial sequence
  Synthetic
tgctacccggccgc

SEQ ID NO: 118 - DNA, 13 bp, Artificial sequence
  Synthetic
caggagggcggcg

SEQ ID NO: 119 - DNA, 41 bp, Artificial sequence
  Synthetic
tatcgcgcggccgggtagcactgcgaaagaaaggcacaaac

SEQ ID NO: 120 - DNA, 41 bp, Artificial sequence
  Synthetic
atcccatcgccgccctcctggtgagtgtcattagacctgtc

SEQ ID NO: 121 - DNA, 32 bp, Artificial sequence
  Synthetic
cgccgcgtccctgcaataaacttgccaatggc

SEQ ID NO: 122 - DNA, 28 bp, Artificial sequence
  Synthetic
tatcggccgggtgagtcgcccccttcct

SEQ ID NO: 123 - DNA, 28 bp, Artificial sequence
  Synthetic
ggcgactcacccggccgatatctcaccc

SEQ ID NO: 124 - DNA, 28 bp, Artificial sequence
  Synthetic
tttattgcagggacgcggcggtggtaat

SEQ ID NO: 125 - DNA, 38 bp, Artificial sequence
  Synthetic
aagttggtggctccgcttccgttagcctcccccatctc

SEQ ID NO: 126 - DNA, 37 bp, Artificial sequence
  Synthetic
ccccagggggatccgccaccatggcttcgtacccctg

SEQ ID NO: 127 - DNA, 15 bp, Artificial sequence
  Synthetic
ggtggcggatccccc

SEQ ID NO: 128 - DNA, 18 bp, Artificial sequence
  Synthetic
ggaagcggagccaccaac

SEQ ID NO: 129 - DNA, 35 bp, Artificial sequence
  Synthetic
aagttggtggctccgcttccggcaccgggcttgcg

SEQ ID NO: 130 - DNA, 35 bp, Artificial sequence
  Synthetic
aaggtaccgagctcgaattcgggtaggggaggcgc

SEQ ID NO: 131 - DNA, 18 bp, Artificial sequence
  Synthetic
gaattcgagctcggtacc

SEQ ID NO: 132 - DNA, 17 bp, Artificial sequence
  Synthetic
ggctagtctcgtgatcg

SEQ ID NO: 133 - DNA, 1793 bp, Artificial sequence
  Synthetic
tatcgatcacgagactagccgcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgatcagttagcctcccccatctcccgggcaaacgtgcgcgccaggtcgcagatcgtcggtatggagccgggggtggtgacgtgggtctggaccatcccggaggtaagttgcagcagggcgtcccggcagccggcgggcgattggtcgtaatccaggataaagacgtgcatgggacggaggcgtttggccaagacgtccaaggcccaggcaaacacgttgtacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccaatcctcccgccacgacccgccgccctgcagataccgcaccgtattggcaagcagcccgtaaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacctgccagtaagtcatcggctcgggtacgtagacgatatcgtcgcgcgaacccagggccaccagcagttgcgtggtggtggttttccccatcccgtgaggaccgtctatataaacccgcagtagcgtgggcattttctgctccaggcggacttccgtggcttcttgctgccggcgagggcgcaacgccgtacgtcggttgctatggccgcgagaacgcgcagcctggtcgaacgcagacgcgtgttgatggcaggggtacgaagccatagggccgggattctcctccacgtcaccagcctgcttcagcagagagaagttggtggctccgcttcc

SEQ ID NO: 134 - DNA, 19 bp, Artificial sequence
  Synthetic
cttgtcattaccaccgccg

SEQ ID NO: 135 - DNA, 19 bp, Artificial sequence
  Synthetic
cttgtcattaccaccgccg

SEQ ID NO: 136 - DNA, 19 bp, Artificial sequence
  Synthetic
cggcggtggtaatgacaag

SEQ ID NO: 137 - DNA, 20 bp, Artificial sequence
  Synthetic
ccgatatctcaccctggtcg

SEQ ID NO: 138 - DNA, 40 bp, Artificial sequence
  Synthetic
ggacgcggcggtggtaatgacaagcgcccagataacaatg

SEQ ID NO: 139 - DNA, 40 bp, Artificial sequence
  Synthetic
ccggccgatatctcaccctggtcgaggcggtgttgtgtgg

SEQ ID NO: 140 - DNA, 40 bp, Artificial sequence
  Synthetic
cattgttatctgggcgcttgtcattaccaccgccgcgtcc

SEQ ID NO: 141 - DNA, 40 bp, Artificial sequence
  Synthetic
ccacacaacaccgcctcgaccagggtgagatatcggccgg

SEQ ID NO: 142 - DNA, 51 bp, Artificial sequence
  Synthetic
acactctttccctacacgacgctcttccgatctgaccagggtgagatatcg

SEQ ID NO: 143 - DNA, 50 bp, Artificial sequence
  Synthetic
gtgactggagttcagacgtgtgctcttccgatcttgtcattaccaccgcc

SEQ ID NO: 144 - DNA, 63 bp, Artificial sequence
  Synthetic
  misc_feature (34)..(45): Wherein N at positions 34 through 45 is defined as any nucleotide
acactctttccctacacgacgctcttccgatctnnnnnnnnnnnngaccagggtgagatatcg

SEQ ID NO: 145 - DNA, 63 bp, Artificial sequence
  Synthetic
  misc_feature (35)..(46): Wherein N at positions 35 through 46 is defined as any nucleotide
gtgactggagttcagacgtgtgctcttccgatctnnnnnnnnnnnnttgtcattaccaccgcc

SEQ ID NO: 146 - DNA, 459 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 147 - DNA, 66 bp, Artificial sequence
  Synthetic
ggaagcggagccaccaacttctctctgctgaagcaggctggtgacgtggaggagaatcccggccct

SEQ ID NO: 148 - DNA, 308 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccag

SEQ ID NO: 149 - DNA, 151 bp, Homo sapiens
ggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 150 - DNA, 150 bp, Artificial sequence; identical to SEQ ID NO: 61
  Synthetic

SEQ ID NO: 151 - DNA, 158 bp, Artificial sequence; identical to SEQ ID NO: 62
  Synthetic

SEQ ID NO: 152 - DNA, 158 bp, Artificial sequence
  Synthetic
gtgagtcgcccccttcctctgctccgagagggaaatgggaattagggtggtggcagagcccaaagaggccttgtaggccatggtaaggcatttctattactaacagttttattttactttgtctttatcctaaaatgccattggcaagtttattgcag

SEQ ID NO: 153 - DNA, 158 bp, Artificial sequence
  Synthetic
gtgagtcgcccccttcctctgctccgagagggaaatgggaattagggtggtggcagagcccaaagaggccttgtaggccatggtactaacataaggcgtttctgtgttttgttttgctttgtctttatcctaaaatgccattggcaagtttattgcag

SEQ ID NO: 154 - DNA, 158 bp, Artificial sequence
  Synthetic
gtgagtcgcccccttcctctgctccgagagggaaatgggaattagggtggtggcagagcccaaagaggccttactaacatgtaggccatggtaaggcgtttctgtgttttgttttgctttgtctttatcctaaaatgccattggcaagtttattgcag

SEQ ID NO: 155 - DNA, 150 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaaatgcccttggcaagtttcttgcag

SEQ ID NO: 156 - DNA, 150 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaaatgccatgggcgagtttattgcag

SEQ ID NO: 157 - DNA, 150 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaaatgccatttgcaagtttcttgcag

SEQ ID NO: 158 - DNA, 25 bp, Artificial sequence
  Synthetic
tcgaccagggtgagatatcggccgg

SEQ ID NO: 159 - DNA, 25 bp, Artificial sequence
  Synthetic
ggacgcggcggtggtaatgacaagc

SEQ ID NO: 160 - DNA, 10 bp, Artificial sequence
  Synthetic
tttgttccgt

SEQ ID NO: 161 - DNA, 10 bp, Artificial sequence
  Synthetic
gggtggttta

SEQ ID NO: 162 - DNA, 10 bp, Artificial sequence
  Synthetic
gtaggtaggt

SEQ ID NO: 163 - DNA, 10 bp, Artificial sequence
  Synthetic
ttcgttctgc

SEQ ID NO: 164 - DNA, 10 bp, Artificial sequence
  Synthetic
ggtaagtagg

SEQ ID NO: 165 - DNA, 10 bp, Artificial sequence
  Synthetic
ggttagttta

SEQ ID NO: 166 - DNA, 10 bp, Artificial sequence
  Synthetic
ttcgtaggta

SEQ ID NO: 167 - DNA, 10 bp, Artificial sequence
  Synthetic
ggtccactag

SEQ ID NO: 168 - DNA, 10 bp, Artificial sequence
  Synthetic
ttctgttcct

SEQ ID NO: 169 - DNA, 10 bp, Artificial sequence
  Synthetic
tcgttcctta

SEQ ID NO: 170 - DNA, 10 bp, Artificial sequence
  Synthetic
gggatggggt

SEQ ID NO: 171 - DNA, 10 bp, Artificial sequence
  Synthetic
gtttgggggt

SEQ ID NO: 172 - DNA, 10 bp, Artificial sequence
  Synthetic
tatagggggg

SEQ ID NO: 173 - DNA, 10 bp, Artificial sequence
  Synthetic
ggggttggga

SEQ ID NO: 174 - DNA, 10 bp, Artificial sequence
  Synthetic
tttcctgatg

SEQ ID NO: 175 - DNA, 10 bp, Artificial sequence
  Synthetic
tgtttagtta

SEQ ID NO: 176 - DNA, 10 bp, Artificial sequence
  Synthetic
ttcttagtta

SEQ ID NO: 177 - DNA, 10 bp, Artificial sequence
  Synthetic
gttaggtata

SEQ ID NO: 178 - DNA, 10 bp, Artificial sequence
  Synthetic
taatagttta

SEQ ID NO: 179 - DNA, 10 bp, Artificial sequence
  Synthetic
ttcgtttggg

SEQ ID NO: 180 - DNA, 157 bp, Artificial sequence
  Synthetic
gtgagtcgcccccttcctctgctcctgggcgtggagagggaaatgggaattagggtggtggcagagcccaaagaggccttgtaggccatggtaaggcatttctatgttttattttactttgtcttatcctaaaatgccattggcaagtttattgcag

SEQ ID NO: 181 - DNA, 149 bp, Artificial sequence
  Synthetic
gtgagtcgcccccttcctctgctccgagagggaaatgggaattagggtggtggcagagcccaaagaggccttgtaggccatggtaaggcatttctatgttttattttactttgtctttatcctaaatgccattggcaagtttattgcag

SEQ ID NO: 182 - DNA, 100 bp, Artificial sequence; identical to SEQ ID NO: 6
  Synthetic

SEQ ID NO: 183 - DNA, 200 bp, Artificial sequence
  Synthetic
tcgaccagggtgagatatcggccgggtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcagggacgcggcggtggtaatgacaagc

SEQ ID NO: 184 - DNA, 200 bp, Artificial sequence
  Synthetic
aacatctacaccacacaacaccgcctcgaccagggtgagatatcggccgggtgagtcgcccccttaggccttgtaggccatggtaaggcatttctatgttttattttactttgtctttatcctaaaatgccattggcaagtttattgcagggacgcggcggtggtaatgacaagcgcccagataacaatgggcatgcctt

SEQ ID NO: 185 - DNA, 200 bp, Artificial sequence
  Synthetic
tcgaccagggtgagatatcggccggccgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcccggacgcggcggtggtaatgacaagc

SEQ ID NO: 186 - DNA, 200 bp, Artificial sequence
  Synthetic
ccagggtgagatatcggccgggtgagtcgcccccttcctctgctccgagagggaaatgggaattagggtggtggcagagcccaaagaggccttgtaggccatggtaaggctactaacaatttctatgttttattttactttgtctttatcctaaaatgccattggcaagtttattgcagggacgcggcggtggtaatgac

SEQ ID NO: 187 - DNA, 200 bp, Artificial sequence
  Synthetic
tcgaccagggtgagatatcggccgggtgagtcgcccccttcctctgctccgagagggaaatgggaattagggtggtggcagagcccaaagaggccttgtaggccatggtaaggcatttctatgttttattttactttgtctttatcctaaaatgccattggcacctttattgcagggacgcggcggtggtaatgacaagc

SEQ ID NO: 188 - DNA, 200 bp, Artificial sequence
  Synthetic
tcgaccagggtgagatatcggccgggtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcggggacgcggcggtggtaatgacaagc

SEQ ID NO: 189 - DNA, 150 bp, Artificial sequence; identical to SEQ ID NO: 61
  Synthetic

SEQ ID NO: 190 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL